(19) United States
(12) Patent Application Publication
(10) Pub. No.: US 2025/0145601 A1
(43) Pub. Date: May 8, 2025

(54) NOVEL BICYCLIC COMPOUNDS AS RAD51 INHIBITORS

(71) Applicant: SATYA THERAPEUTICS, INC., Lewes, DE (US)

(72) Inventors: Sanjita SASMAL, Hyderabad (IN); Venkatesham BOORGU, Hyderabad (IN); Shankar CHITHALURI, Hyderabad (IN)

(21) Appl. No.: 18/837,659

(22) PCT Filed: Feb. 20, 2023
(86) PCT No.: PCT/IB2023/051530

§ 371 Date: Aug. 12, 2024

(30) Foreign Application Priority Data
Feb. 22, 2022 (IN) 202241009273

(51) Int. Cl. C07D 413/14; A61K 31/4365; A61K 31/4375; A61K 31/444; A61K 31/4709; A61K 31/4725; A61K 31/496; A61K 31/506; A61K 31/519; A61K 31/5377; A61P 35/00; C07D 401/04; C07D 401/14; C07D 405/14; C07D 471/04; C07D 495/04

(52) U.S. Cl. CPC C07D 413/14; A61K 31/4365; A61K 31/4375; A61K 31/444; A61K 31/4709; A61K 31/4725; A61K 31/496; A61K 31/506; A61K 31/519; A61K 31/5377; A61P 35/00; C07D 401/04; C07D 401/14; C07D 405/14; C07D 471/04; C07D 495/04

(57) ABSTRACT

The present invention relates to novel bicyclic compounds of formula (I) as inhibitors of RAD51 inhibitors, their pharmaceutically acceptable salts, solvates, polymorphs, tautomers, optical and geometric isomers thereof.

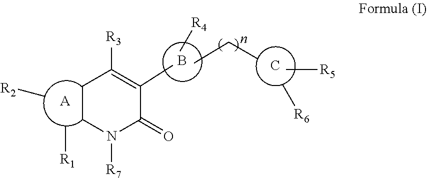

### FIELD OF INVENTION

The present invention relates to novel bicyclic compounds of formula (I) as inhibitors of RAD51 inhibitors, their pharmaceutically acceptable salts, solvates, polymorphs, tautomers, optical and geometric isomers thereof.

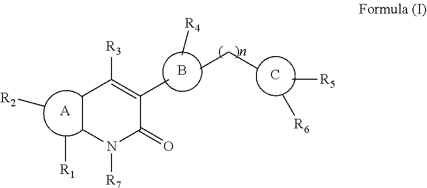

The present invention also relates to a process for the manufacture of novel bicyclic compounds of formula (I), pharmaceutical compositions containing them, and their use in the treatment of cancer.

### BACKGROUND OF THE INVENTION

RAD51 gene encodes for RAD51 recombinase, a protein essential for DNA double strand break resolution via homologous recombination. Diseases associated with RAD51 include Fanconi Anemia, Complementation Group R and Mirror Movements 2. Among its related pathways are Meiosis and Resolution of D-Loop Structures. Gene Ontology (GO) annotations related to this gene include identical protein binding and protein C-terminus binding. An important paralog of this gene is DMC1.

RAD51 family members are highly similar to bacterial RecA and Saccharomyces cerevisiae Rad51, and are known to be involved in the homologous recombination and repair of DNA. This protein can interact with the ssDNA-binding protein RPA and RAD52, and it is thought to play roles in homologous pairing and strand transfer of DNA. This protein is also found to interact with BRCA1 and BRCA2, which may be important for the cellular response to DNA damage. BRCA2 is shown to regulate both the intracellular localization and DNA-binding ability of this protein. RAD51 function is severely compromised during BRCA2 inactivation and may lead to genomic instability and tumorigenesis.

In humans, RAD51 is a 339-amino acid protein that plays a major role in homologous recombination of DNA during double strand break repair. In this process, an ATP dependent DNA strand exchange takes place in which a template strand invades base-paired strands of homologous DNA molecules. RAD51 is involved in the search for homology and strand pairing stages of the process.

Unlike other proteins involved in DNA metabolism, the RecA/Rad51 family forms a helical nucleoprotein filament on DNA. [Structure. 14 (6): 983-92] This protein can interact with the ssDNA-binding protein RPA, BRCA2, PALB2 [Nature Structural & Molecular Biology. 17 (10): 1247-54.] and RAD52.

RAD51 protein has a central role in homologous recombinational repair. RAD51 catalyses strand transfer between a broken sequence and its undamaged homologue to allow re-synthesis of the damaged region in an error free manner. This is crucial for preventing spontaneous mutation during DNA replication and repair.

Numerous studies report that RAD51 is over-expressed in different cancers. In many of these studies, elevated expression of RAD51 is correlated with decreased patient survival.

WO 2016/140971 A1 of The Regents of The University of California discloses compounds having the following formulae:

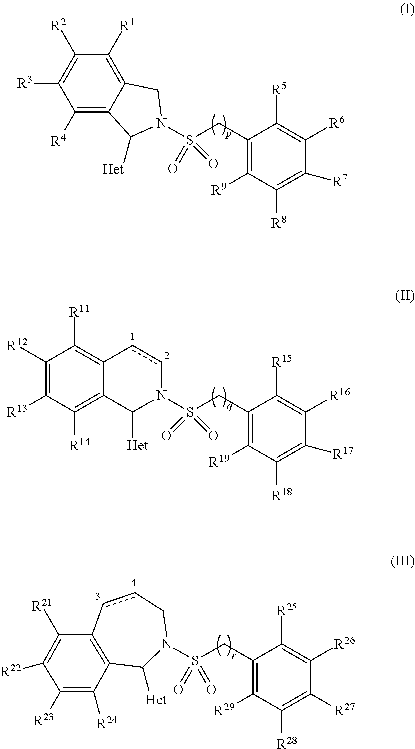

as RAD51 inhibitors.

WO 2021/116999 A1 of Italian Institute of Technology discloses compound of formula (la):

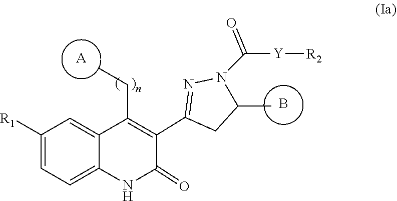

J. Med. Chem. 2020, 63, 2588-2619 discloses compounds of formula as RAD 5 inhibitors.

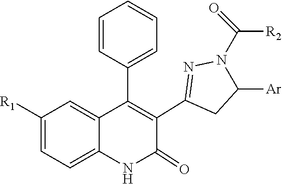

Treatment of cancer continues to be a challenge though several drugs that have been approved with different mechanism. In view of this, there is unmet need for new drugs that can treat such diseases more effectively. We herein describe novel bicyclic compounds as RAD51 inhibitors.

### OBJECTIVE OF THE INVENTION

An objective of the present invention is to provide novel bicyclic compounds of formula (I) as RAD51 inhibitors, their pharmaceutically acceptable salts, solvates, polymorphs, tautomers, optical and geometric isomers thereof.

Yet another objective of the present invention is to provide a process for the manufacture of novel bicyclic compounds of formula (I), pharmaceutical compositions containing them.

Still another objective of the present invention is to provide novel bicyclic compounds of formula (I) for use in the treatment of cancer.

### SUMMARY OF THE INVENTION

The present invention provides novel bicyclic compounds of formula (I) their pharmaceutically acceptable salts, solvates, polymorphs, tautomers, optical and geometric isomers thereof.

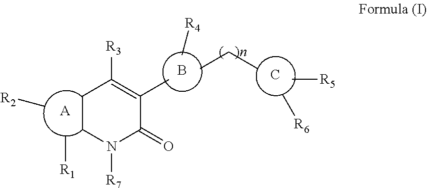

ring A is selected from 6-10 membered aryl, 5-10 membered heteroaryl, 6-10 membered cyclic ring system and 3-10 membered heterocyclyl;
ring B is selected from 6-10 membered aryl, 5-10 membered heteroaryl and 3-10 membered heterocyclyl;
ring C is selected from 6-10 membered aryl, 5-10 membered heteroaryl and 3-10 membered heterocyclyl;
wherein either of A or B or C is optionally substituted by one or more, identical or different substituents;
R¹ and R² is present or absent and when present is selected from the group consisting of hydrogen, halogen, alkyl, alkoxy, —C(O)alkyl, cycloalkyl, heterocyclyl, aryl, heteroaryl; wherein any of the group is optionally substituted by one or more, identical or different substituents;
R³ is selected from the group consisting of hydrogen, halogen, amino, nitro, alkyl, haloalkyl, alkoxy, hydroxyalkyl, cycloalkyl, heterocyclyl, heteroaryl;
R⁴ is selected from the group consisting of hydrogen, alkyl, —C(O)—R^{4a} wherein any of the group is optionally substituted by one or more, identical or different substituents;
R^{4a} is selected from the group consisting of hydrogen, alkyl, haloalkyl, cycloalkyl, heterocyclyl; wherein any of the group is optionally substituted by one or more, identical or different substituents;
R⁵ and R⁶ are same or different and each individually is selected from the group consisting of hydrogen, halogen, amino, nitro, alkyl, haloalkyl, cycloalkyl, alkoxy, acylamino, alkylamino, dialkylamino, heterocyclyl; wherein any of the group is optionally substituted by one or more, identical or different substituents;
R⁷ is selected from the group consisting of hydrogen, alkyl and “n” is 0 to 2.

In another embodiment, the present invention provides process for the preparation of novel bicyclic compounds of formula (I).

In yet another embodiment, the present invention provides pharmaceutical composition comprising novel bicyclic compounds of the formula (I) and processes for preparing thereof.

In yet further another embodiment, the present invention provides use of novel bicyclic compounds of formula (I) and pharmaceutically acceptable derivatives, salts and regioisomers thereof, including mixtures thereof in all ratios as a medicament, by inhibiting RAD51 in treating diseases such as cancer.

### DETAILED DESCRIPTION OF THE INVENTION

According to another embodiment, specifically provided are compounds of formula (I), in which the ring A along with the attached ring is specifically represented by

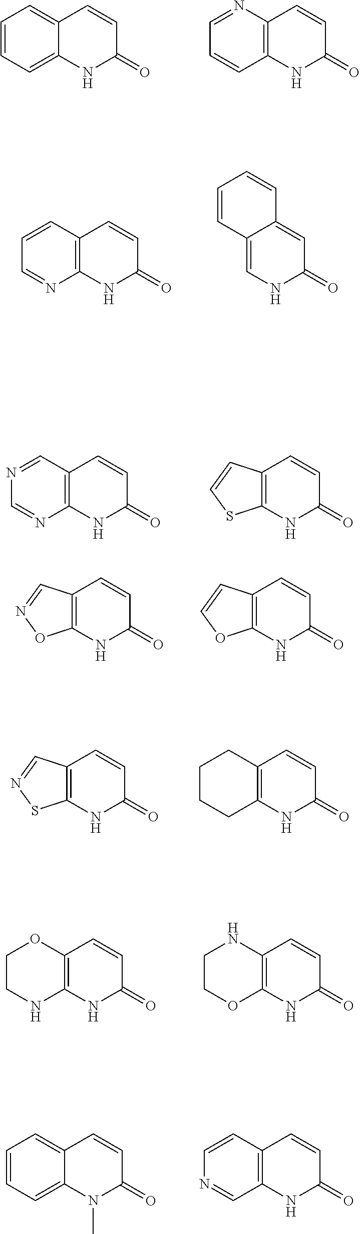

In another embodiment the present invention provides novel compounds of formula (Ia)

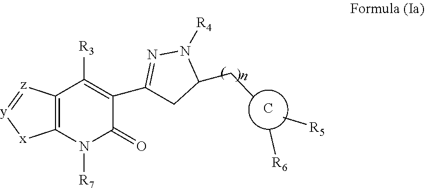

or a pharmaceutically acceptable salt or a pharmaceutically acceptable regioisomer thereof; wherein,
“x” is selected from O, S or N;
“y” and “z” independently are selected from N, CR′, wherein R′ is selected from hydrogen, halogen, alkyl;
ring C is selected from 6-10 membered aryl, 5-10 membered heteroaryl and 3-10 membered heterocyclyl, optionally substituted by one or more, identical or different substituents;
R³ is selected from the group consisting of hydrogen, halogen, amino, nitro, alkyl, haloalkyl, alkoxy, hydroxyalkyl, cycloalkyl, heterocyclyl, heteroaryl;
R⁴ is selected from the group consisting of hydrogen, alkyl, —C(O)—R^{4a} wherein any of the group is optionally substituted by one or more, identical or different substituents;
R^{4a} is selected from the group consisting of hydrogen, alkyl, haloalkyl, cycloalkyl, heterocyclyl; wherein any of the group is optionally substituted by one or more, identical or different substituents;
R⁵ and R⁶ are same or different and each individually is selected from the group consisting of hydrogen, halogen, amino, nitro, alkyl, haloalkyl, cycloalkyl, alkoxy, acylamino, alkylamino, dialkylamino, heterocyclyl; wherein any of the group is optionally substituted by one or more, identical or different substituents;
R⁷ is selected from the group consisting of hydrogen, alkyl and
“n” is 0 to 2.

In another aspect the present invention relates to novel compounds of formula (Ib)

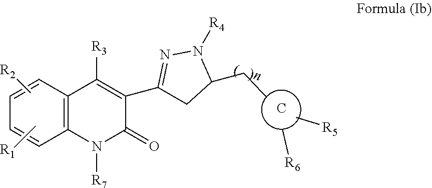

or a pharmaceutically acceptable salt or a pharmaceutically acceptable regioisomer thereof; wherein,
ring C is selected from 6-10 membered aryl, 5-10 membered heteroaryl and 3-10 membered heterocyclyl, optionally substituted by one or more, identical or different substituents;
R¹ and R² is present or absent and when present is selected from the group consisting of hydrogen, halogen, alkyl, alkoxy, —C(O)alkyl, cycloalkyl, heterocyclyl, aryl, heteroaryl; wherein any of the group is optionally substituted by one or more, identical or different substituents;
R³ is selected from the group consisting of hydrogen, halogen, amino, nitro, alkyl, haloalkyl, alkoxy, hydroxyalkyl, cycloalkyl, heterocyclyl, heteroaryl;
R⁴ is selected from the group consisting of hydrogen, alkyl, —C(O)—R^{4a} wherein any of the group is optionally substituted by one or more, identical or different substituents;
R^{4a} is selected from the group consisting of hydrogen, alkyl, haloalkyl, cycloalkyl, heterocyclyl; wherein any of the group is optionally substituted by one or more, identical or different substituents;
R⁵ and R⁶ are same or different and each individually is selected from the group consisting of hydrogen, halogen, amino, nitro, alkyl, haloalkyl, cycloalkyl, alkoxy, acylamino, alkylamino, dialkylamino heterocyclyl; wherein any of the group is optionally substituted by one or more, identical or different substituents;
R⁷ is selected from the group consisting of hydrogen, alkyl and
“n” is 0 to 2.

In another aspect the present invention relates to novel compounds of formula (Ic)

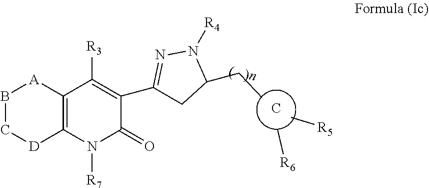

or a pharmaceutically acceptable salt or a pharmaceutically acceptable regioisomer thereof; wherein,
ring C is selected from 6-10 membered aryl, 5-10 membered heteroaryl and 3-10 membered heterocyclyl, optionally substituted by one or more, identical or different substituents;
“A”, “B”, “C”, and “D” independently are selected from O, S, N, C(R′)₂, or any of the two A and B or C and D represent CR′ form a double bond, wherein R′ is absent or when present is selected from hydrogen, halogen, alkyl;
R³ is selected from the group consisting of hydrogen, halogen, amino, nitro, alkyl, haloalkyl, alkoxy, hydroxyalkyl, cycloalkyl, heterocyclyl, heteroaryl;
R⁴ is selected from the group consisting of hydrogen, alkyl, —C(O)—R^{4a} wherein any of the group is optionally substituted by one or more, identical or different substituents;
R^{4a} is selected from the group consisting of hydrogen, alkyl, haloalkyl, cycloalkyl, heterocyclyl, wherein any of the group is optionally substituted by one or more, identical or different substituents;
R⁵ and R⁶ are same or different and each individually is selected from the group consisting of hydrogen, halogen, amino, nitro, alkyl, haloalkyl, cycloalkyl, alkoxy, acylamino, alkylamino, dialkylamino, heterocyclyl; wherein any of the group is optionally substituted by one or more, identical or different substituents;
R⁷ is selected from the group consisting of hydrogen, alkyl and
“n” is 0 to 2.

In another aspect the present invention relates to novel compounds of formula (Id)

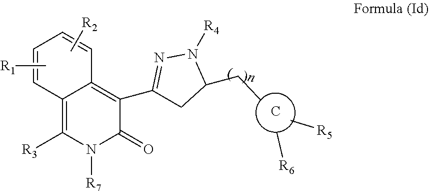

or a pharmaceutically acceptable salt or a pharmaceutically acceptable regioisomer thereof; wherein,
ring C is selected from 6-10 membered aryl, 5-10 membered heteroaryl and 3-10 membered heterocyclyl, optionally substituted by one or more, identical or different substituents;
R¹ and R² is present or absent and when present is selected from the group consisting of hydrogen, halogen, alkyl, alkoxy, —C(O)alkyl, cycloalkyl, heterocyclyl, aryl, heteroaryl, wherein any of the group is optionally substituted by one or more, identical or different substituents;
R³ is selected from the group consisting of hydrogen, halogen, amino, nitro, alkyl, haloalkyl, hydroxyalkyl, cycloalkyl, heterocyclyl, heteroaryl;
R⁴ is selected from the group consisting of hydrogen, alkyl, —C(O)—R^{4a} wherein any of the group is optionally substituted by one or more, identical or different substituents;
R^{4a} is selected from the group consisting of hydrogen, alkyl, haloalkyl, cycloalkyl, heterocyclyl, wherein any of the group is optionally substituted by one or more, identical or different substituents;
R⁵ and R⁶ are same or different and each individually is selected from the group consisting of hydrogen, halogen, amino, nitro, alkyl, haloalkyl, cycloalkyl, alkoxy, acylamino, alkylamino, dialkylamino, heterocyclyl; wherein any of the group is optionally substituted by one or more, identical or different substituents;
R⁷ is selected from the group consisting of hydrogen, alkyl and
“n” is 0 to 2.

According to another embodiment, specifically provided are compounds of formula (I), in ring C is specifically represented by

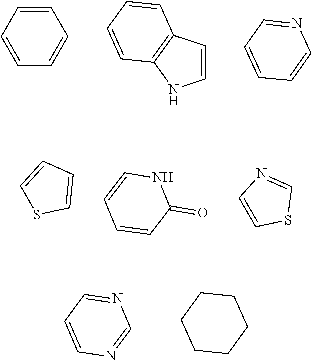

Without limiting the scope of present invention, the following definitions are provided in order to understand the detailed description of the present invention.

The term “Alkyl” as used herein refers and is not limited to a hydrocarbon chain that may be a linear or branched chain, containing the indicated number of carbon atoms, for example, a C₁-C₁₂ alkyl group may have from 1 to 12 (inclusive) carbon atoms in it. Examples of C₁-C₁₂alkyl groups include, but are not limited to, methyl, ethyl, propyl, isopropyl, butyl, isobutyl, sec-butyl, tert-butyl, pentyl, isopentyl, neopentyl, hexyl, and isohexyl. An alkyl group can be unsubstituted or substituted with one or more suitable groups.

The term “alkenyl” as used herein refers and is not limited to a linear, branched unsaturated C₁-C₆ hydrocarbyl group containing a double bond, but are not limited to ethenyl, propenyl, butenyl. An alkenyl group can be unsubstituted or substituted with one or more suitable groups.

The term “alkynyl” as used herein refers and is not limited to a linear, branched unsaturated C₁-C₆ hydrocarbyl group containing a triple bond, but are not limited to acetylenyl, propynyl, butynyl. An alkynyl group can be unsubstituted or substituted with one or more suitable groups.

The term “Amino” as used herein refers and is not limited to an —N— group, the nitrogen atom of said group being attached to a hydrogen, alkyl, cycloalkyl, aryl, heterocyclyl or any suitable groups. An amino group can be unsubstituted or substituted with one or more of the suitable groups.

The term “Aryl” as used herein refers and is not limited to an optionally substituted monocylic, bicyclic or polycyclic aromatic carbocyclic ring system of about 6 to 14 carbon atoms. Examples of a C₆-C₁₄ aryl group include, but are not limited to phenyl, naphthyl, biphenyl, anthryl, tetrahydronaphthyl, fluorenyl, indanyl, biphenylenyl, and acenaphthyl. Aryl group which can be unsubstituted or substituted with one or more suitable groups.

The term “Halogen” or “halo” includes fluorine, chlorine, bromine or iodine. “Hydroxy” refers to —OH group.

The term “cycloalkyl” as used herein refers and is not limited to a non-aromatic, saturated or partially saturated, monocyclic or polycyclic 3 to 10 member ring system. Exemplary cycloalkyl groups include cyclopropyl, cyclobutyl, cyclopentyl, cyclohexyl, and the like. A cycloalkyl group can be unsubstituted or substituted with one or more suitable groups.

The term “Heterocyclyl” as used herein refers and is not limited to a non-aromatic, saturated or partially saturated, monocyclic or polycyclic ring system of 3 to 10 member having at least one heteroatom or heterogroup selected from O, N, S, S(O), S(O)₂, NH and C(O). Exemplary heterocycloalkyl groups include tetrahydrofuranyl, tetrahydropyranyl, pyrrolidinyl, piperdinyl, piperazinyl, morpholinyl, thiomorpholinyl, 1,3-dioxolanyl, 1,4-dioxanyl, azetadine, oxetane, thietane and the like. A heterocycloalkyl group can be unsubstituted or substituted with one or more suitable groups.

The term “Heteroaryl” as used herein refers and is not limited to an unsaturated, monocyclic, bicyclic, or polycyclic aromatic ring system containing at least one heteroatom selected from oxygen, sulphur and nitrogen. Examples of C₅-C₁₀ heteroaryl groups include furan, thiophene, indole, azaindole, oxazole, thiazole, thiadiazole, isoxazole, isothiazole, imidazole, N-methylimidazole, pyridine, pyrimidine, pyrazine, pyrrole, N-methylpyrrole, pyrazole, N-methylpyrazole, 1,3,4-oxadiazole, 1,2,4-triazole, 1-methyl-1,2,4-triazole, 1H-tetrazole, 1-methyltetrazole, benzoxazole, benzothiazole, benzofuran, benzisoxazole, benzimidazole, N-methylbenzimidazole, azabenzimidazole, indazole, quinazoline, quinoline, and isoquinoline. Bicyclic heteroaryl groups include those where a phenyl, pyridine, pyrimidine or pyridazine ring is fused to a 5 or 6-membered monocyclic heterocyclyl ring having one or two nitrogen atoms in the ring, one nitrogen atom together with either one oxygen or one sulfur atom in the ring, or one O or S ring atom. A heteroaryl group can be unsubstituted or substituted with one or more suitable groups.

“Optionally substituted or substituted” as used herein means that at least one hydrogen atom of the optionally substituted group has been substituted with suitable substitutions as exemplified but not limited to halogen, nitro, cyano, hydroxy, hydroxyl alkyl, amino, oxo (═O), thiooxo (=S), —N(C₁-C₃alkyl)(C₁-C₆alkyl), —N(C₁-C₃alkyl)C(O)(C₁-C₆alkyl), —NHC(O)(C₁-C₆alkyl), —NHC(O)(cycloalkyl), —NHC(O)(aryl), —NHC(O)(heterocyclyl), —NHC(O)(heteroaryl), —NHC(O)H, —C(O)NH₂, —C(O)NH(C₁-C₆alkyl), —C(O)NH(cycloalkyl), —C(O)NH(heterocyclyl), —C(O)NH(heteroaryl), —C(O)N(C₁-C₆alkyl)(C₁-C₆alkyl), —S(O)NH(C₁-C₆alkyl), —S(O)₂NH(C₁-C₆alkyl), —S(O)NH(cycloalkyl), —S(O)₂NH(cycloalkyl), carboxy, —C(O)O(C₁-C₆alkyl), —C(O)(C₁-C₆alkyl), ═N—OH, substituted or unsubstituted alkyl, substituted or unsubstituted haloalkyl, substituted or unsubstituted alkoxy, substituted or unsubstituted haloalkoxy, substituted or unsubstituted alkenyl, substituted or unsubstituted alkynyl, substituted or unsubstituted aryl, substituted or unsubstituted arylalkyl, substituted or unsubstituted cycloalkyl, substituted or unsubstituted cycloalkenylalkyl, substituted or unsubstituted cycloalkenyl, substituted or unsubstituted amino, substituted or unsubstituted heteroaryl, substituted or unsubstituted heterocyclyl.

The particular compounds of the present invention without departing from the scope of the definitions given under compounds of formula (I) and particular compounds emanated from formula (I) are summarized herein below table encompassing the entirety of the scope of compounds within compound of formula (I).
1. 6-Chloro-3-(5-(4-methoxyphenyl)-1-propionyl-4,5-dihydro-1H-pyrazol-3-yl)-4-methylquinolin-2(1H)-one
2. 6-Chloro-3-(1-(cyclopropanecarbonyl)-5-(4-methoxyphenyl)-4,5-dihydro-1H-pyrazol-3-yl)-4-methylquinolin-2(1H)-one
3. 6-chloro-3-(5-(4-methoxyphenyl)-1-(tetrahydro-2H-pyran-4-carbonyl)-4,5-dihydro-1H-pyrazol-3-yl)-4-methylquinolin-2(1H)-one
4. 6-chloro-3-(5-(4-methoxyphenyl)-1-(tetrahydrofuran-3-carbonyl)-4,5-dihydro-1H-pyrazol-3-yl)-4-methylquinolin-2(1H)-one
5. 6-Chloro-3-(5-(4-methoxyphenyl)-1-methyl-4,5-dihydro-1H-pyrazol-3-yl)-4-methylquinolin-2(1H)-one
6. 6-Chloro-4-methyl-3-(5-(1-methyl-1H-indol-6-yl)-1-propionyl-4,5-dihydro-1H-pyrazol-3-yl)quinolin-2(1H)-one
7. 6-chloro-3-(5-(4-methoxyphenyl)-1-propionyl-4,5-dihydro-1H-pyrazol-3-yl)-4-methylquinolin-2(1H)-one
8. N-(4-(3-(6-chloro-4-methyl-2-oxo-1,2-dihydroquinolin-3-yl)-1-propionyl-4,5-dihydro-1H-pyrazol-5-yl)phenyl)propionamide
9. 3-(5-(4-Methoxyphenyl)-1-propionyl-4,5-dihydro-1H-pyrazol-3-yl)-4-methyl-1,5-naphthyridin-2(1H)-one
10. 4-Methyl-3-(5-(1-methyl-1H-indol-5-yl)-1-propionyl-4,5-dihydro-1H-pyrazol-3-yl)quinolin-2(1H)-one
11. 3-(5-(4-Methoxyphenyl)-1-propionyl-4,5-dihydro-1H-pyrazol-3-yl)-4-methylquinolin-2(1H)-one
12. 4-Methyl-3-(5-(1-methyl-1H-indol-3-yl)-1-propionyl-4,5-dihydro-1H-pyrazol-3-yl)quinolin-2(1H)-one
13. 3-(5-(4-Methoxyphenyl)-1-propionyl-4,5-dihydro-1H-pyrazol-3-yl)-4-methyl-1,8-naphthyridin-2(1H)-one
14. 6-(5-(4-Methoxyphenyl)-1-propionyl-4,5-dihydro-1H-pyrazol-3-yl)-5-methylpyrido-[2,3-d]pyrimidin-7(8H)-one
15. 6-bromo-3-(5-(4-methoxyphenyl)-1-propionyl-4,5-dihydro-1H-pyrazol-3-yl)-4-methylquinolin-2(1H)-one
16. 6-chloro-3-(5-(4-methoxyphenyl)-1-propionyl-4,5-dihydro-1H-pyrazol-3-yl)-4-methyl-1,5-naphthyridin-2(1H)-one
17. 6-Chloro-3-(5-(4-methoxyphenyl)-1-propionyl-1H-pyrazol-3-yl)quinolin-2(1H)-one
18. 6-chloro-3-(5-(4-methoxyphenyl)-1-propionyl-4,5-dihydro-1H-pyrazol-3-yl)-1,4-dimethylquinolin-2(1H)-one
19. 4-(5-(4-methoxyphenyl)-1-propionyl-4,5-dihydro-1H-pyrazol-3-yl)isoquinolin-3(2H)-one
20. 5-(5-(4-Methoxyphenyl)-1-propionyl-4,5-dihydro-1H-pyrazol-3-yl)-4-methylthieno-[2,3-b]pyridin-6(7H)-one
21. 5-(5-(6-methoxypyridin-3-yl)-1-propionyl-4,5-dihydro-1H-pyrazol-3-yl)-4-methylthieno[2,3-b]pyridin-6(7H)-one
22. 5-(5-(4-methoxyphenyl)-1-propionyl-4,5-dihydro-1H-pyrazol-3-yl)thieno[2,3-b]pyridin-6(7H)-one
23. 6-chloro-4-cyclopropyl-3-(5-(4-methoxyphenyl)-1-propionyl-4,5-dihydro-1H-pyrazol-3-yl)quinolin-2(1H)-one
24. 6-chloro-4-methyl-3-(5-(1-methyl-1H-indol-3-yl)-1-propionyl-4,5-dihydro-1H-pyrazol-3-yl)quinolin-2(1H)-one
25. 5-(5-(5-methoxypyridin-2-yl)-1-propionyl-4,5-dihydro-1H-pyrazol-3-yl)-4-methylthieno[2,3-b]pyridin-6(7H)-one
26. 4-methyl-5-(5-(6-oxo-1,6-dihydropyridin-3-yl)-1-propionyl-4,5-dihydro-1H-pyrazol-3-yl)thieno[2,3-b]pyridin-6(7H)-one
27. 5-(5-(4-fluorophenyl)-1-propionyl-4,5-dihydro-1H-pyrazol-3-yl)-4-methylthieno[2,3-b]pyridin-6(7H)-one
28. 5-(5-(4-chlorophenyl)-1-propionyl-4,5-dihydro-1H-pyrazol-3-yl)-4-methylthieno[2,3-b]pyridin-6(7H)-one
29. 5-(5-(5-methoxythiophen-2-yl)-1-propionyl-4,5-dihydro-1H-pyrazol-3-yl)-4-methylthieno[2,3-b]pyridin-6(7H)-one
30. 5-(5-(2-methoxythiazol-5-yl)-1-propionyl-4,5-dihydro-1H-pyrazol-3-yl)-4-methylthieno[2,3-b]pyridin-6(7H)-one
31. 5-(5-(5-bromothiophen-3-yl)-1-propionyl-4,5-dihydro-1H-pyrazol-3-yl)-4-methylthieno[2,3-b]pyridin-6(7H)-one
32. 4-methyl-5-(5-phenyl-1-propionyl-4,5-dihydro-1H-pyrazol-3-yl)thieno[2,3-b]pyridin-6(7H)-one
33. 4-methyl-5-(1-propionyl-5-(thiophen-3-yl)-4,5-dihydro-1H-pyrazol-3-yl)thieno[2,3-b]pyridin-6(7H)-one
34. 5-(5-(4-bromothiophen-2-yl)-1-propionyl-4,5-dihydro-1H-pyrazol-3-yl)-4-methylthieno[2,3-b]pyridin-6(7H)-one
35. 4-methyl-5-(1-propionyl-5-(pyridin-4-yl)-4,5-dihydro-1H-pyrazol-3-yl)thieno[2,3-b]pyridin-6(7H)-one
36. 4-methyl-5-(1-propionyl-5-(pyridin-3-yl)-4,5-dihydro-1H-pyrazol-3-yl)thieno[2,3-b]pyridin-6(7H)-one
37. 4-methyl-5-(1-propionyl-5-(p-tolyl)-4,5-dihydro-1H-pyrazol-3-yl)thieno[2,3-b]pyridin-6(7H)-one
38. 4-methyl-5-(5-(4-(4-methylpiperazin-1-yl)phenyl)-1-propionyl-4,5-dihydro-1H-pyrazol-3-yl)thieno[2,3-b]pyridin-6(7H)-one
39. 5-(5-(4-(dimethylamino)phenyl)-1-propionyl-4,5-dihydro-1H-pyrazol-3-yl)-4-methylthieno[2,3-b]pyridin-6(7H)-one
40. 3-chloro-5-(5-(4-methoxyphenyl)-1-propionyl-4,5-dihydro-1H-pyrazol-3-yl)-4-methylthieno[2,3-b]pyridin-6(7H)-one
41. 5-(5-(5-methylthiophen-2-yl)-1-propionyl-4,5-dihydro-1H-pyrazol-3-yl)-4-methylthieno[2,3-b]pyridin-6(7H)-one
42. 4-methyl-5-(1-propionyl-5-(4-(trifluoromethyl)phenyl)-4,5-dihydro-1H-pyrazol-3-yl)thieno[2,3-b]pyridin-6(7H)-one
43. 5-(5-(4-(difluoromethyl)phenyl)-1-propionyl-4,5-dihydro-1H-pyrazol-3-yl)-4-methylthieno[2,3-b]pyridin-6(7H)-one
44. 5-(5-(4-ethylphenyl)-1-propionyl-4,5-dihydro-1H-pyrazol-3-yl)-4-methylthieno[2,3-b]pyridin-6(7H)-one
45. 4-methyl-5-(5-(6-methylpyridin-3-yl)-1-propionyl-4,5-dihydro-1H-pyrazol-3-yl)thieno[2,3-b]pyridin-6(7H)-one
46. 4-methyl-5-(1-propionyl-5-(4-(pyrrolidin-1-yl)phenyl)-4,5-dihydro-1H-pyrazol-3-yl)thieno[2,3-b]pyridin-6(7H)-one
47. 5-(5-(4-cyclopropylphenyl)-1-propionyl-4,5-dihydro-1H-pyrazol-3-yl)-4-methylthieno[2,3-b]pyridin-6(7H)-one
48. 4-methyl-5-(5-(2-methylthiazol-4-yl)-1-propionyl-4,5-dihydro-1H-pyrazol-3-yl)thieno[2,3-b]pyridin-6(7H)-one
49. 5-(5-(4-isopropylphenyl)-1-propionyl-4,5-dihydro-1H-pyrazol-3-yl)-4-methylthieno[2,3-b]pyridin-6(7H)-one
50. 5-(5-(4,6-dimethylpyridin-3-yl)-1-propionyl-4,5-dihydro-1H-pyrazol-3-yl)-4-methylthieno[2,3-b]pyridin-6(7H)-one
51. 4-methyl-5-(5-(2-methylpyrimidin-5-yl)-1-propionyl-4,5-dihydro-1H-pyrazol-3-yl)thieno[2,3-b]pyridin-6(7H)-one
52. 5-(5-(5-chloro-6-methylpyridin-3-yl)-1-propionyl-4,5-dihydro-1H-pyrazol-3-yl)-4-methylthieno[2,3-b]pyridin-6(7H)-one
53. 5-(5-(4-methoxy-6-methylpyridin-3-yl)-1-propionyl-4,5-dihydro-1H-pyrazol-3-yl)-4-methylthieno[2,3-b]pyridin-6(7H)-one
54. 5-(5-(5,6-dimethylpyridin-3-yl)-1-propionyl-4,5-dihydro-1H-pyrazol-3-yl)-4-methylthieno[2,3-b]pyridin-6(7H)-one
55. 5-(5-(5-fluoro-6-methylpyridin-3-yl)-1-propionyl-4,5-dihydro-1H-pyrazol-3-yl)-4-methylthieno[2,3-b]pyridin-6(7H)-one
56. 5-(5-(4-chloro-6-methylpyridin-3-yl)-1-propionyl-4,5-dihydro-1H-pyrazol-3-yl)-4-methylthieno[2,3-b]pyridin-6(7H)-one
57. 4-methyl-5-(5-(2-methylthiazol-5-yl)-1-propionyl-4,5-dihydro-1H-pyrazol-3-yl)thieno[2,3-b]pyridin-6(7H)-one
58. 5-(5-cyclohexyl-1-propionyl-4,5-dihydro-1H-pyrazol-3-yl)-4-methylthieno[2,3-b]pyridin-6(7H)-one
59. 6-chloro-4-methyl-3-(1-propionyl-5-(p-tolyl)-4,5-dihydro-1H-pyrazol-3-yl)quinolin-2(1H)-one
60. 4-methyl-3-(1-propionyl-5-(p-tolyl)-4,5-dihydro-1H-pyrazol-3-yl)quinolin-2(1H)-one
61. 6-chloro-4-methyl-3-(1-propionyl-5-(p-tolyl)-4,5-dihydro-1H-pyrazol-3-yl)-1,5-naphthyridin-2(1H)-one
62. 5-(1-acetyl-5-(p-tolyl)-4,5-dihydro-1H-pyrazol-3-yl)-4-methyl-7,7a-dihydrothieno[2,3-b]pyridin-6(7H)-one
63. 5-(1-isobutyryl-5-(p-tolyl)-4,5-dihydro-1H-pyrazol-3-yl)-4-methyl-7,7a-dihydrothieno[2,3-b]pyridin-6(7H)-one
64. 4-methyl-5-(5-(p-tolyl)-1-(2,2,2-trifluoroacetyl)-4,5-dihydro-1H-pyrazol-3-yl)-7,7a-dihydrothieno[2,3-b]pyridin-6(7H)-one
65. 5-(5-(2-chloro-4-methylphenyl)-1-propionyl-4,5-dihydro-1H-pyrazol-3-yl)-4-methylthieno[2,3-b]pyridin-6(7H)-one
66. 5-(5-(2-fluoro-4-methylphenyl)-1-propionyl-4,5-dihydro-1H-pyrazol-3-yl)-4-methylthieno[2,3-b]pyridin-6(7H)-one
67. 6-chloro-4-methyl-3-(1-propionyl-5-(p-tolyl)-4,5-dihydro-1H-pyrazol-3-yl)-1,8-naphthyridin-2(1H)-one
68. 5-(5-(4-(fluoromethyl)phenyl)-1-propionyl-4,5-dihydro-1H-pyrazol-3-yl)-4-methylthieno[2,3-b]pyridin-6(7H)-one
69. 4-methyl-3-(1-propionyl-5-(p-tolyl)-4,5-dihydro-1H-pyrazol-3-yl)-1,5-naphthyridin-2(1H)-one
70. 2,4-dimethyl-5-(1-propionyl-5-(p-tolyl)-4,5-dihydro-1H-pyrazol-3-yl)thieno[2,3-b]pyridin-6(7H)-one
71. 5-(5-benzyl-1-propionyl-4,5-dihydro-1H-pyrazol-3-yl)-4-methylthieno[2,3-b]pyridin-6(7H)-one
72. 2-chloro-4-methyl-5-(1-propionyl-5-(p-tolyl)-4,5-dihydro-1H-pyrazol-3-yl)thieno[2,3-b]pyridin-6(7H)-one
73. 4-methyl-3-(1-propionyl-5-(p-tolyl)-4,5-dihydro-1H-pyrazol-3-yl)-1,8-naphthyridin-2(1H)-one
74. 4-methyl-3-(1-propionyl-5-(p-tolyl)-4,5-dihydro-1H-pyrazol-3-yl)-1,7-naphthyridin-2(1H)-one
75. 4,7-dimethyl-3-(1-propionyl-5-(p-tolyl)-4,5-dihydro-1H-pyrazol-3-yl)quinolin-2(1H)-one
76. 4,5-dimethyl-3-(1-propionyl-5-(p-tolyl)-4,5-dihydro-1H-pyrazol-3-yl)quinolin-2(1H)-one
77. 4-methyl-7-morpholino-3-(1-propionyl-5-(p-tolyl)-4,5-dihydro-1H-pyrazol-3-yl)quinolin-2(1H)-one
78. 4,8-dimethyl-3-(1-propionyl-5-(p-tolyl)-4,5-dihydro-1H-pyrazol-3-yl)-1,7-naphthyridin-2(1H)-one
79. 6-chloro-4-methyl-3-(1-propionyl-5-(p-tolyl)-4,5-dihydro-1H-pyrazol-3-yl)-1,7-naphthyridin-2(1H)-one′
80. 7-chloro-4-methyl-3-(1-propionyl-5-(p-tolyl)-4,5-dihydro-1H-pyrazol-3-yl)quinolin-2(1H)-one
81. 4,6-dimethyl-3-(1-propionyl-5-(p-tolyl)-4,5-dihydro-1H-pyrazol-3-yl)-1,7-naphthyridin-2(1H)-one
82. 4-methyl-7-(4-methylpiperazin-1-yl)-3-(1-propionyl-5-(p-tolyl)-4,5-dihydro-1H-pyrazol-3-yl)quinolin-2(1H)-one
83. 7-fluoro-4-methyl-3-(1-propionyl-5-(p-tolyl)-4,5-dihydro-1H-pyrazol-3-yl)quinolin-2(1H)-one
84. 4,7-dimethyl-3-(1-propionyl-5-(p-tolyl)-4,5-dihydro-1H-pyrazol-3-yl)-1,8-naphthyridin-2(1H)-one
85. 4-methyl-3-(1-propionyl-5-(p-tolyl)-4,5-dihydro-1H-pyrazol-3-yl)-1,6-naphthyridin-2(1H)-one
86. 4-methyl-7-morpholino-3-(1-propionyl-5-(p-tolyl)-4,5-dihydro-1H-pyrazol-3-yl)-1,8-naphthyridin-2(1H)-one
87. 7-(2-methoxyethoxy)-4-methyl-3-(1-propionyl-5-(p-tolyl)-4,5-dihydro-1H-pyrazol-3-yl)-1,8-naphthyridin-2(1H)-one
   or a pharmaceutically acceptable salt thereof or a pharmaceutically acceptable regioisomer thereof.

Few compounds of the present invention were separated into their pure enantiomers which are shown in the table below:

TABLE 1

| Compound | Structure & IUPAC Name | |
|---|---|---|
| No. | (racemic) | Structure & IUPAC Name (Pure Enantiomer) |
| 20 | 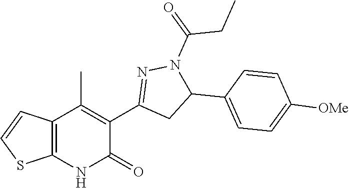 [SMILES: CCC(=O)N1N=C(c2c(C)c3ccsc3[nH]c2=O)CC1c1ccc(OC)cc1] | 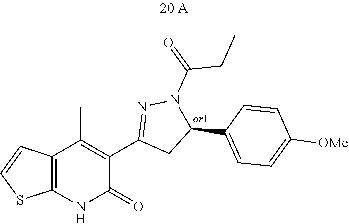 [SMILES: CCC(=O)N1N=C(c2c(C)c3ccsc3[nH]c2=O)C[C@@H]1c1ccc(OC)cc1] |
| | 5-(5-(4-methoxyphenyl)-1-propionyl-4,5-dihydro-1H- | rel-(R)-5-(5-(4-methoxyphenyl)-1-propionyl-4,5-dihydro-1H- |
| | pyrazol-3-yl)-4-methylthieno[2,3-b]pyridin-6(7H)-one | pyrazol-3-yl)-4-methylthieno[2,3-b]pyridin-6(7H)-one |
| | | 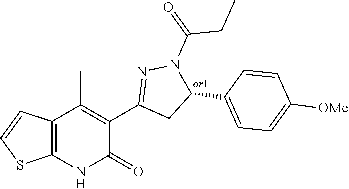 [SMILES: CCC(=O)N1N=C(c2c(C)c3ccsc3[nH]c2=O)C[C@H]1c1ccc(OC)cc1] |
| | | rel-(S)-5-(5-(4-methoxyphenyl)-1-propionyl-4,5- |
| | | dihydro-1H-pyrazol-3-yl)-4-methylthieno[2,3- |
| | | b]pyridin-6(7H)-one |
| 28 | 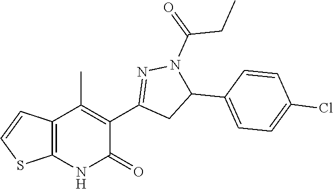 [SMILES: CCC(=O)N1N=C(c2c(C)c3ccsc3[nH]c2=O)CC1c1ccc(Cl)cc1] | 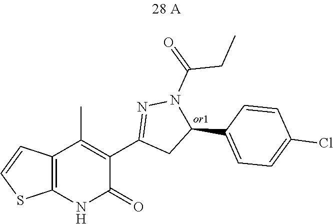 [SMILES: CCC(=O)N1N=C(c2c(C)c3ccsc3[nH]c2=O)C[C@@H]1c1ccc(Cl)cc1] |
| | 5-(5-(4-chlorophenyl)-1-propionyl-4,5-dihydro-1H- | rel-(R)-5-(5-(4-chlorophenyl)-1-propionyl-4,5-dihydro-1H- |
| | pyrazol-3-yl)-4-methylthieno[2,3-b]pyridin-6(7H)-one | pyrazol-3-yl)-4-methylthieno[2,3-b]pyridin-6(7H)-one |
| | | 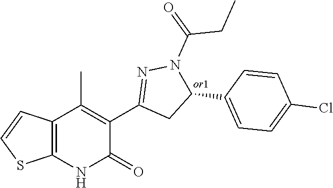 [SMILES: CCC(=O)N1N=C(c2c(C)c3ccsc3[nH]c2=O)C[C@H]1c1ccc(Cl)cc1] |
| | | rel-(S)-5-(5-(4-chlorophenyl)-1-propionyl-4,5- |
| | | dihydro-1H-pyrazol-3-yl)-4-methylthieno[2,3- |
| | | b]pyridin-6(7H)-one |
| 33 | 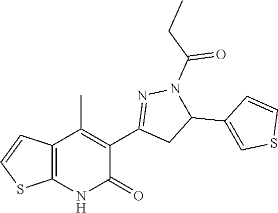 [SMILES: CCC(=O)N1N=C(c2c(C)c3ccsc3[nH]c2=O)CC1c1ccsc1] | 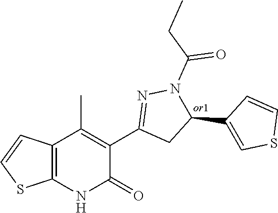 [SMILES: CCC(=O)N1N=C(c2c(C)c3ccsc3[nH]c2=O)C[C@@H]1c1ccsc1] |
| | 4-methyl-5-(1-propionyl-5-(thiophen-3-yl)-4,5- | rel-(R)-4-methyl-5-(1-propionyl-5-(thiophen-3-yl)- |
| | dihydro-1H-pyrazol-3-yl)thieno[2,3-b]pyridin-6(7H)-one | 4,5-dihydro-1H-pyrazol-3-yl)thieno[2,3-b]pyridin-6(7H)-one |
| | | 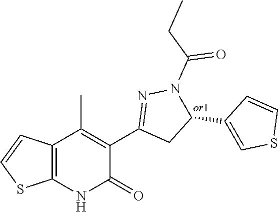 [SMILES: CCC(=O)N1N=C(c2c(C)c3ccsc3[nH]c2=O)C[C@H]1c1ccsc1] |
| | | rel-(S)-4-methyl-5-(1-propionyl-5-(thiophen-3-yl)- |
| | | 4,5-dihydro-1H-pyrazol-3-yl)thieno[2,3-b]pyridin- |
| | | 6(7H)-one |
| 37 | 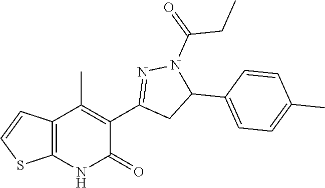 [SMILES: CCC(=O)N1N=C(c2c(C)c3ccsc3[nH]c2=O)CC1c1ccc(C)cc1] | 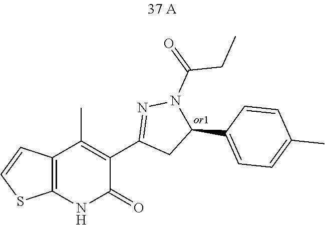 [SMILES: CCC(=O)N1N=C(c2c(C)c3ccsc3[nH]c2=O)C[C@@H]1c1ccc(C)cc1] |
| | 4-methyl-5-(1-propionyl-5-(p-tolyl)-4,5-dihydro- | rel-(R)-4-methyl-5-(1-propionyl-5-(p-tolyl)-4,5- |
| | 1H-pyrazol-3-yl)thieno[2,3-b]pyridin-6(7H)-one | dihydro-1H-pyrazol-3-yl)thieno[2,3-b]pyridin-6(7H)-one |
| | | 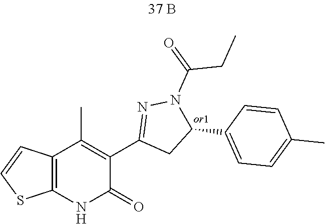 [SMILES: CCC(=O)N1N=C(c2c(C)c3ccsc3[nH]c2=O)C[C@H]1c1ccc(C)cc1] |
| | | rel-(S)-4-methyl-5-(1-propionyl-5-(p-tolyl)-4,5- |
| | | dihydro-1H-pyrazol-3-yl)thieno[2,3-b]pyridin- |
| | | 6(7H)-one |
| 45 | 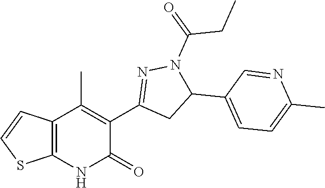 [SMILES: CCC(=O)N1N=C(c2c(C)c3ccsc3[nH]c2=O)CC1c1ccc(C)nc1] | 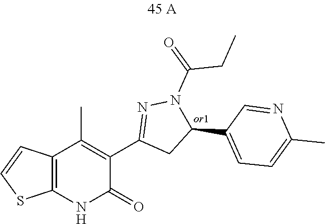 [SMILES: CCC(=O)N1N=C(c2c(C)c3ccsc3[nH]c2=O)C[C@@H]1c1ccc(C)nc1] |
| | 4-methyl-5-(5-(6-methylpyridin-3-yl)-1- | rel-(R)-4-methyl-5-(5-(6-methylpyridin-3-yl)-1- |
| | propionyl-4,5-dihydro-1H-pyrazol-3-yl)thieno[2,3- | propionyl-4,5-dihydro-1H-pyrazol-3-yl)thieno[2,3- |
| | b]pyridin-6(7H)-one | b]pyridin-6(7H)-one |
| | | 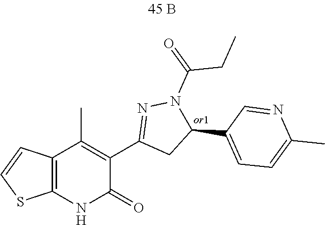 [SMILES: CCC(=O)N1N=C(c2c(C)c3ccsc3[nH]c2=O)C[C@@H]1c1ccc(C)nc1] |
| | | rel-(S)-4-methyl-5-(5-(6-methylpyridin-3-yl)-1- |
| | | propionyl-4,5-dihydro-1H-pyrazol-3-yl)thieno[2,3- |
| | | b]pyridin-6(7H)-one |
| 60 | 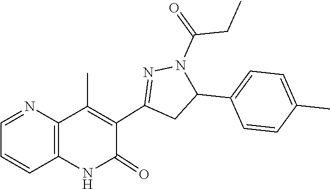 [SMILES: CCC(=O)N1N=C(c2c(C)c3ncccc3[nH]c2=O)CC1c1ccc(C)cc1] | 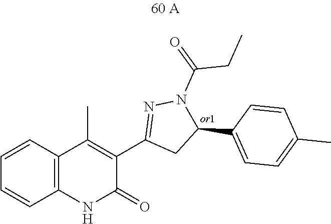 [SMILES: CCC(=O)N1N=C(c2c(C)c3ccccc3[nH]c2=O)C[C@@H]1c1ccc(C)cc1] |
| | 4-methyl-3-(1-propionyl-5-(p-tolyl)-4,5-dihydro- | rel-(R)-4-methyl-3-(1-propionyl-5-(p-tolyl)-4,5- |
| | 1H-pyrazol-3-yl)quinolin-2(1H)-one | dihydro-1H-pyrazol-3-yl)quinolin-2-(1H)-one |
| | | 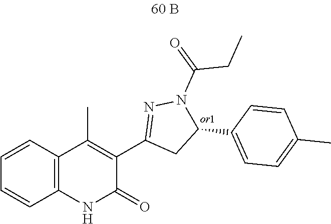 [SMILES: CCC(=O)N1N=C(c2c(C)c3ccccc3[nH]c2=O)C[C@H]1c1ccc(C)cc1] |
| | | rel-(S)-4-methyl-3-(1-propionyl-5-(p-tolyl)-4,5- |
| | | dihydro-1H-pyrazol-3-yl)quinolin-2(1H)-one |
| 66 | 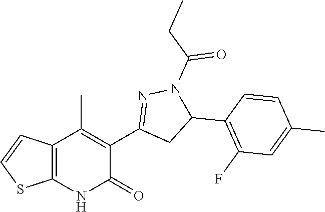 [SMILES: CCC(=O)N1N=C(c2c(C)c3ccsc3[nH]c2=O)CC1c1ccc(C)cc1F] | 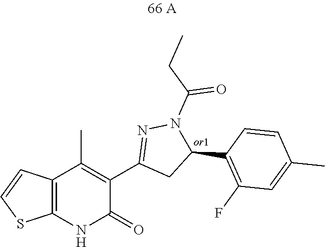 [SMILES: CCC(=O)N1N=C(c2c(C)c3ccsc3[nH]c2=O)C[C@@H]1c1ccc(C)cc1F] |
| | 5-(5-(2-fluoro-4-methylphenyl)-1-propionyl-4,5-dihydro-1H- | rel-(R)-5-(5-(2-fluoro-4-methylphenyl)-1-propionyl-4,5-dihydro- |
| | pyrazol-3-yl)-4-methylthieno[2,3-b]pyridin-6(7H)-one | 1H-pyrazol-3-yl)-4-methylthieno[2,3-b]pyridin-6(7H)-one |
| | | 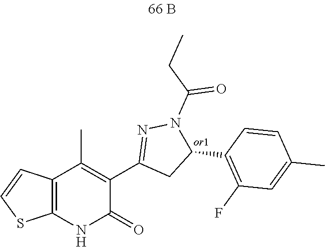 [SMILES: CCC(=O)N1N=C(c2c(C)c3ccsc3[nH]c2=O)C[C@H]1c1ccc(C)cc1F] |
| | | rel-(S)-5-(5-(2-fluoro-4-methylphenyl)-1- |
| | | propionyl-4,5-dihydro-1H-pyrazol-3-yl)-4- |
| | | methylthieno[2,3-b]pyridin-6(7H)-one |
| 74 | 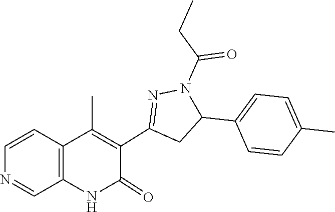 [SMILES: CCC(=O)N1N=C(c2c(C)c3ccncc3[nH]c2=O)CC1c1ccc(C)cc1] | 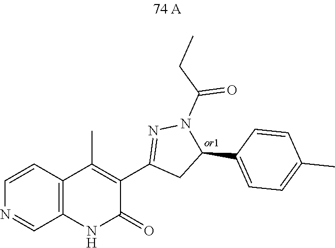 [SMILES: CCC(=O)N1N=C(c2c(C)c3ccncc3[nH]c2=O)C[C@@H]1c1ccc(C)cc1] |
| | 4-methyl-3-(1-propionyl-5-(p-tolyl)-4,5-dihydro- | rel-(R)-4-methyl-3-(1-propionyl-5-(p-tolyl)-4,5- |
| | 1H-pyrazol-3-yl)-1,7-naphthyridin-2(1H)-one | dihydro-1H-pyrazol-3-yl)-1,7-naphthyridin-2(1H)-one |
| | | 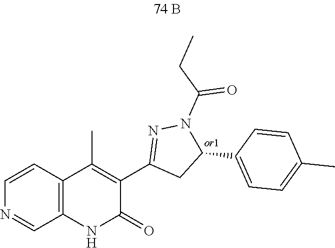 [SMILES: CCC(=O)N1N=C(c2c(C)c3ccncc3[nH]c2=O)C[C@H]1c1ccc(C)cc1] |
| | | rel-(S)-4-methyl-3-(1-propionyl-5-(p-tolyl)-4,5- |
| | | dihydro-1H-pyrazol-3-yl)-1,7-naphthyridin-2(1H)-one |
| 78 | 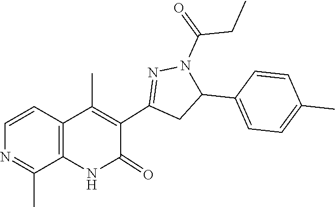 [SMILES: CCC(=O)N1N=C(c2c(C)c3ccnc(C)c3[nH]c2=O)CC1c1ccc(C)cc1] | 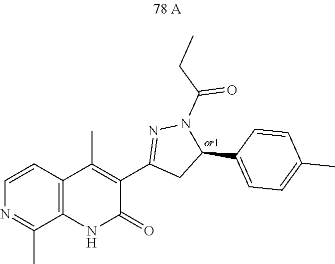 [SMILES: CCC(=O)N1N=C(c2c(C)c3ccnc(C)c3[nH]c2=O)C[C@@H]1c1ccc(C)cc1] |
| | 4,8-dimethyl-3-(1-propionyl-5-(p-tolyl)-4,5-dihydro-1H- | rel-(R)-4,8-dimethyl-3-(1-propionyl-5-(p-tolyl)-4,5- |
| | pyrazol-3-yl)-1,7-naphthyridin-2(1H)-one | dihydro-1H-pyrazol-3-yl)-1,7-naphthyridin-2(1H)-one |
| | | 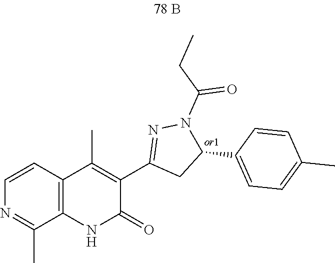 [SMILES: CCC(=O)N1N=C(c2c(C)c3ccnc(C)c3[nH]c2=O)C[C@H]1c1ccc(C)cc1] |
| | | rel-(S)-4,8-dimethyl-3-(1-propionyl-5-(p-tolyl)-4,5- |
| | | dihydro-1H-pyrazol-3-yl)-1,7-naphthyridin-2(1H)-one |
| 80 | 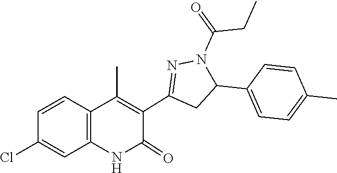 [SMILES: CCC(=O)N1N=C(c2c(C)c3ccc(Cl)cc3[nH]c2=O)CC1c1ccc(C)cc1] | 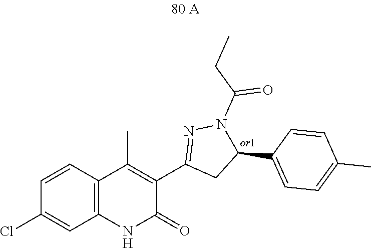 [SMILES: CCC(=O)N1N=C(c2c(C)c3ccc(Cl)cc3[nH]c2=O)C[C@@H]1c1ccc(C)cc1] |
| | 7-chloro-4-methyl-3-(1-propionyl-5-(p-tolyl)-4,5- | rel-(R)-7-chloro-4-methyl-3-(1-propionyl-5-(p- |
| | dihydro-1H-pyrazol-3-yl)quinolin-2(1H)-one | tolyl)-4,5-dihydro-1H-pyrazol-3-yl)quinolin-2(1H)-one |
| | | 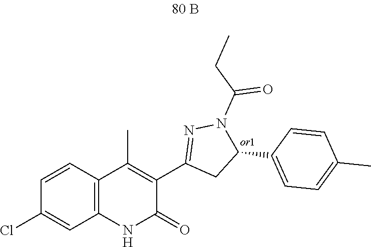 [SMILES: CCC(=O)N1N=C(c2c(C)c3ccc(Cl)cc3[nH]c2=O)C[C@H]1c1ccc(C)cc1] |
| | | rel-(S)-7-chloro-4-methyl-3-(1-propionyl-5-(p- |
| | | tolyl)-4,5-dihydro-1H-pyrazol-3-yl)quinolin-2(1H)-one |
| 82 | 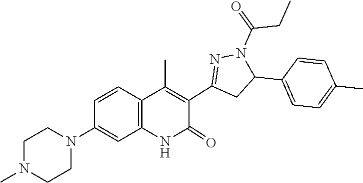 | 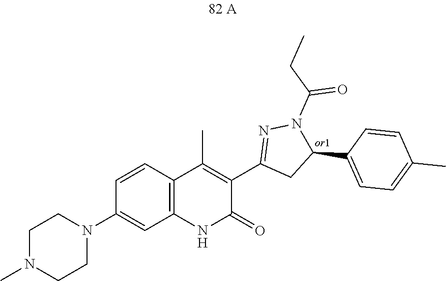 |
| | 4-methyl-7-(4-methylpiperazin-1-yl)-3-(1-propionyl-5- | rel-(R)-4-methyl-7-(4-methylpiperazin-1-yl)-3-(1-propionyl-5-(p-tolyl)- |
| | (p-tolyl)-4,5-dihydro-1H-pyrazol-3-yl)quinolin-2(1H)-one | 4,5-dihydro-1H-pyrazol-3-yl)quinolin-2(1H)-one |
| | | 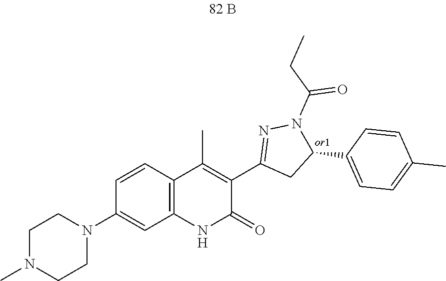 |
| | | rel-(S)-4-methyl-7-(4-methylpiperazin-1-yl)-3-(1-propionyl-5- |
| | | (p-tolyl)-4,5-dihydro-1H-pyrazol-3-yl)quinolin-2(1H)-one |

Further embodiments of the invention on includes use of compounds of formula (I) or pharmaceutically acceptable derivatives, salts and regio-isomers thereof, including mixtures thereof in all ratios as a medicament.

Unless specifically indicated, the general formula of compound (I) shall encompass tautomers and all stereo, optical and geometrical isomers (e.g. enantiomers, diastereomers, E/Z isomers, etc.) and racemates thereof as well as mixtures in different proportions of the separate enantiomers, mixtures of diastereomers, or mixtures of any of the foregoing forms where such isomers and enantiomers exist, as well as salts, including pharmaceutically acceptable salts thereof and solvates thereof such as for instance hydrates including solvates and hydrates of the free compound or solvates and hydrates of a salt of the compound.

In general, substantially pure stereoisomers can be obtained according to synthetic principles known to a person skilled in the field, e.g. by separation of corresponding mixtures, by using stereochemically pure starting materials and/or by stereoselective synthesis. It is known in the art how to prepare optically active forms, such as by resolution of racemic forms or by synthesis, e.g. starting from optically active starting materials and/or by using chiral reagents.

The present invention further provides a pharmaceutical composition comprising at least one compound according to formula (I) and/or pharmaceutically usable derivatives, salts, tautomers and regioisomers thereof, including mixtures thereof in all ratios, optional additional second active ingredient, and excipients.

The term “pharmaceutically acceptable salt” or “pharmaceutically acceptable derivatives” is taken to mean an active ingredient, which comprises a compound of the formula (I) in the form of one of its salts, in particular if this salt form imparts improved pharmacokinetic properties on the active ingredient compared with the free form of the active ingredient or any other salt form of the active ingredient used earlier. The pharmaceutically acceptable salt form of the active ingredient can also provide this active ingredient for the first time with a desired pharmacokinetic property which it did not have earlier and can even have a positive influence on the pharmacodynamics of this active ingredient with respect to its therapeutic efficacy in the body.

The term “regioisomer” or “regioisomers” refers to the positional isomers, which is a category of structural isomers, wherein the position or the substituent changes position on the parent structure. Herein the term regioisomer without departing from the scope of compound of formula (I) inherently includes all regioisomers either as a pure regioisomer or mixture of two or more regioisomers thereof. Since the pharmaceutical activity of the regioisomers of the compounds of the present invention may differ, it may be desirable to use the regioisomers. In these cases the regioisomers can be separated at any of the possible stage either as an intermediate or as an end product by the process well known to the person skilled in the art or even employed as such in the synthesis.

The term “tautomer” or “tautomers” refers to the compound of formula (I) of the present invention wherein any hydrogen atom is replaced by a hydroxyl group on a carbon with a double bond. The present invention includes all possible tautomeric forms.

Pharmaceutical formulations can be adapted for administration via any desired suitable method, for example by oral (including buccal or sublingual), rectal, nasal, topical (including buccal, sublingual or transdermal), vaginal or parenteral (including subcutaneous, intramuscular, intravenous or intradermal) methods. Such formulations can be prepared using all processes known in the pharmaceutical art by, for example, combining the active ingredient with the excipient(s) or adjuvant(s).

Pharmaceutical formulations adapted for oral administration can be administered as separate units, such as, for example, capsules or tablets; powders or granules; solutions or suspensions in aqueous or non-aqueous liquids; edible foams or foam foods; or oil-in-water liquid emulsions or water-in-oil liquid emulsions.

For example, in the case of oral administration as tablet or capsule, the active-ingredient component can be combined with an oral, non-toxic and pharmaceutically acceptable inert excipient, such as, for example, ethanol, glycerol, water and the like. Powders are prepared by comminuting the compound to a suitable fine size and mixing it with a pharmaceutical excipient comminuted in a similar manner, such as, for example, an edible carbohydrate, such as, for example, starch or mannitol. A flavour, preservative, dispersant and dye may likewise be present.

Capsules are produced by preparing a powder mixture as described above and filling shaped gelatine shells therewith. Glidants and lubricants, such as, for example, highly disperse silicic acid, talc, magnesium stearate, calcium stearate or polyethylene glycol in solid form can be added to the powder mixture before the filling operation. A disintegrant or solubiliser, such as, for example, agar-agar, calcium carbonate or sodium carbonate, may likewise be added in order to improve the availability of the medicament after the capsule has been taken.

In addition, if desired or necessary, suitable binders, lubricants and disintegrants as well as dyes can likewise be incorporated into the mixture. Suitable binders include starch, gelatine, natural sugars, such as, for example, glucose or beta-lactose, sweeteners made from maize, natural and synthetic rubber, such as, for example, acacia, tragacanth or sodium alginate, carboxymethylcellulose, polyethylene glycol, waxes, and the like. The lubricants used in these dosage forms include sodium oleate, sodium stearate, magnesium stearate, sodium benzoate, sodium acetate, sodium chloride and the like. The disintegrants include, without being restricted thereto, starch, methylcellulose, agar, bentonite, xanthan gum and the like. The tablets are formulated by, for example, preparing a powder mixture, granulating or dry-pressing the mixture, adding a lubricant and a disintegrant and pressing the entire mixture to give tablets. A powder mixture is prepared by mixing the compound comminuted in a suitable manner with a diluent or a base, as described above, and optionally with a binder, such as, for example, carboxymethylcellulose, an alginate, gelatine or polyvinyl-pyrrolidone, a dissolution retardant, such as, for example, paraffin, an absorption accelerator, such as, for example, a quaternary salt, and/or an absorbant, such as, for example, bentonite, kaolin or dicalcium phosphate. The powder mixture can be granulated by wetting it with a binder, such as, for example, syrup, starch paste, acadia mucilage or solutions of cellulose or polymer materials and pressing it through a sieve. As an alternative to granulation, the powder mixture can be run through a tableting machine, giving lumps of non-uniform shape which are broken up to form granules. The granules can be lubricated by addition of stearic acid, a stearate salt, talc or mineral oil in order to prevent sticking to the tablet casting moulds. The lubricated mixture is then pressed to give tablets. The active ingredients can also be combined with a free-flowing inert excipient and then pressed directly to give tablets without carrying out the granulation or dry-pressing steps. A transparent or opaque protective layer consisting of a shellac sealing layer, a layer of sugar or polymer material and a gloss layer of wax may be present. Dyes can be added to these coatings in order to be able to differentiate between different dosage units.

Oral liquids, such as, for example, solution, syrups and elixirs, can be prepared in the form of dosage units so that a given quantity comprises a pre-specified amount of the compounds. Syrups can be prepared by dissolving the compounds in an aqueous solution with a suitable flavour, while elixirs are prepared using a non-toxic alcoholic vehicle. Suspensions can be formulated by dispersion of the compounds in a non-toxic vehicle. Solubilisers and emulsifiers, such as, for example, ethoxylated isostearyl alcohols and polyoxyethylene sorbitol ethers, preservatives, flavour additives, such as, for example, peppermint oil or natural sweeteners or saccharin, or other artificial sweeteners and the like, can likewise be added.

The dosage unit formulations for oral administration can, if desired, be encapsulated in microcapsules. The formulation can also be prepared in such a way that the release is extended or retarded, such as, for example, by coating or embedding of particulate material in polymers, wax and the like.

The formulations cab be in the form of liposome delivery systems, such as, for example, small unilamellar vesicles, large unilamellar vesicles and multilamellar vesicles. Liposomes can be formed from suitable lipids or phospholipids or both, such as, for example, cholesterol, stearylamine or phosphatidylcholines or the like.

Pharmaceutical formulations adapted for transdermal administration can be administered as independent plasters for extended, close contact with the epidermis of the recipient. Thus, for example, the active ingredient can be delivered from the plaster by iontophoresis, as described in general terms in Pharmaceutical Research, 3(6), 318 (1986).

Pharmaceutical compounds adapted for topical administration can be formulated as ointments, creams, suspensions, lotions, powders, solutions, pastes, gels, sprays, aerosols or oils.

For the treatment of the eye or other external tissue, for example mouth and skin, the formulations are preferably applied as topical ointment or cream. In the case of formulation to give an ointment, the active ingredient can be employed either with a paraffinic or a water-miscible cream base. Alternatively, the active ingredient can be formulated to give a cream with an oil-in-water cream base or a water-in-oil base.

Pharmaceutical formulations adapted for topical application to the eye include eye drops, in which the active ingredient is dissolved or suspended in a suitable carrier, in particular an aqueous solvent.

Pharmaceutical formulations adapted for topical application in the mouth encompass lozenges, pastilles and mouthwashes.

Pharmaceutical formulations adapted for rectal administration can be administered in the form of suppositories or enemas.

Pharmaceutical formulations adapted for nasal administration in which the carrier substance is a solid comprise a coarse powder having a particle size, for example, in the range 20-500 microns, which is administered in the manner in which snuff is taken, i.e. by rapid inhalation via the nasal passages from a container containing the powder held close to the nose. Suitable formulations for administration as nasal spray or nose drops with a liquid as carrier substance encompass active-ingredient solutions in water or oil.

Pharmaceutical formulations adapted for administration by inhalation encompass finely particulate dusts or mists, which can be generated by various types of pressurized dispensers with aerosols, nebulisers or inhalers.

Pharmaceutical formulations adapted for vaginal administration can be administered as pessaries, tampons, creams, gels, pastes, foams or spray formulations. Pharmaceutical formulations adapted for parenteral administration include aqueous and non-aqueous sterile injection solutions comprising antioxidants, buffers, bacteriostatics and solutes, by means of which the formulation is rendered isotonic with the blood of the recipient to be treated; and aqueous and non-aqueous sterile suspensions, which may comprise suspension media and thickeners. The formulations can be administered in single-dose or multidose containers, for example sealed ampoules and vials, and stored in freeze-dried (lyophilised) state, so that only the addition of the sterile carrier liquid, for example water for injection purposes, immediately before use is necessary.

Injection solutions and suspensions prepared in accordance with the recipe can be prepared from sterile powders, granules and tablets.

It goes without saying that, in addition to the above particularly mentioned constituents, the formulations may also comprise other agents usual in the art with respect to the particular type of formulation; thus, for example, formulations which are suitable for oral administration may comprise flavours.

A therapeutically effective amount of a compound of the formula (I) and of the other active ingredient depends on a number of factors, including, for example, the age and weight of the animal, the precise disease condition which requires treatment, and its severity, the nature of the formulation and the method of administration, and is ultimately determined by the treating doctor or vet. However, an effective amount of a compound is generally in the range from 0.1 to 100 mg/kg of body weight of the recipient (mammal) per day and particularly typically in the range from 1 to 10 mg/kg of body weight per day. Thus, the actual amount per day for an adult mammal weighing 70 kg is usually between 70 and 700 mg, where this amount can be administered as an individual dose per day or usually in a series of part-doses (such as, for example, two, three, four, five or six) per day, so that the total daily dose is the same. An effective amount of a salt or solvate or of a physiologically functional derivative thereof can be determined as the fraction of the effective amount of the compound per se.

In a further aspect, the present invention relates to a process for preparing novel compounds of formula (I).

The novel bicyclic compounds of formula (I) may be prepared from readily available starting materials using the following general methods and procedures. It will be appreciated that where typical or preferred experimental conditions (i.e. reaction temperatures, time, moles of reagents, solvents etc.) are given, other experimental conditions can also be used unless otherwise stated. Optimum reaction conditions may vary with the particular reactants or solvents used, but such conditions can be determined by the person skilled in the art, using routine optimization procedures. Moreover, by utilizing the procedures described in detail, one of ordinary skill in the art can prepare additional compounds of the present invention claimed herein. All temperatures are in degrees Celsius (° C.) unless otherwise noted.

In another embodiment of the present invention provides methods useful for preparing the compounds of formula (I) depicted generically in the Schemes given hereunder. One skilled-in-the-art will recognize that any of the Schemes can be adapted to produce the compounds of formula (I) and pharmaceutically accepted salts of compounds of formula (I) according to the present invention. All symbols/variables are as defined hereunder unless otherwise stated.

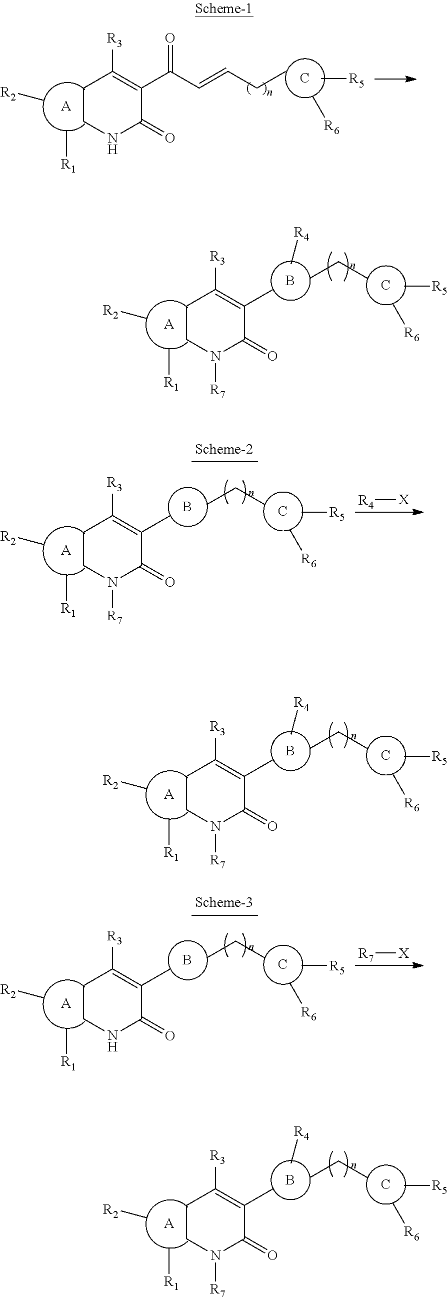

### EXAMPLES

Although the invention has been illustrated by certain of the preceding examples, it is not to be construed as being limited thereby; but rather, the invention encompasses the generic area as hereinbefore disclosed. Various modifications and embodiments can be made without departing from the spirit and scope thereof.

### Example-1: Synthesis of 6-Chloro-3-(5-(4-methoxyphenyl)-1-propionyl-4,5-dihydro-1H-pyrazol-3-yl)-4-methylquinolin-2(1H)-one

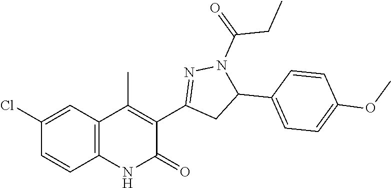 [SMILES: CCC(=O)N1N=C(c2c(C)c3cc(Cl)ccc3[nH]c2=O)CC1c1ccc(OC)cc1]

### Step-a: Synthesis of 1-(2-Amino-5-chlorophenyl)ethan-1-one (1a)

To a stirred solution of 2-amino-5-chlorobenzonitrile (5.0 g, 32.00 mmol) in dry THF (50 mL) at 0° C. was added methyl magnesium bromide (32.8 mL, 96.0 mmol, 3 M in diethyl ether) slowly in dropwise manner for about 15-20 min. The reaction mixture was heated at 60° C. for 16 h and progress of the reaction was monitored by TLC. Reaction mixture was slowly brought to room temperature and cooled to 0° C., quenched with 2N HCl (100 mL) and then extracted with ethyl acetate (2×150 mL). Combined organic layer was washed with water (100 mL), brine (50 mL), dried over Na₂SO₄ and concentrated under reduced pressure to get the crude compound which was purified by column chromatography and eluted with 10-20% ethyl acetate in hexane to afford the titled compound as a solid. Yield: 3.2 g (60%); ¹H-NMR (300 MHz, CDCl₃): ⊐ 7.64 (d, J=2.4 Hz, 1H), 7.22-7.18 (dd, J=2.4 Hz & 6.6 Hz, 1H), 6.60 (d, J=8.7 Hz, 1H), 6.26 (bs, 2H), 2.55 (s, 3H).

### Step-b: Synthesis of 3-Acetyl-6-chloro-4-methylquinolin-2(1H)-one (1b)

To a stirred solution of 1-(2-amino-5-chlorophenyl)ethan-1-one (3.2 g, 18.80 mmol) in toluene (32 mL) at room temperature was added ethyl acetoacetate (3.68 g, 28.30 mmol) and cerium chloride·heptahydrate (3.51 g, 9.40 mmol) and the reaction mixture was refluxed for 16 h. Progress of the reaction was monitored by TLC. The Reaction mixture was brought to room temperature and diluted with water (100 mL), extracted with ethyl acetate (2×75 mL). The combined organic layer was washed with brine (50 mL), dried over Na₂SO₄ and evaporated under reduced pressure to get the crude compound which was purified by flash column chromatography with 40-50% ethyl acetate in hexane as eluent to afford the titled compound as a yellow solid. Yield: 1.0 g (23%); ¹H-NMR (300 MHz, CDCl₃): [symbol] 11.18 (bs, 1H), 7.73 (d, J=2.1 Hz, 1H), 7.50 (dd, J=2.4 Hz & 6.3 Hz, 1H), 7.23 (d, J=8.7 Hz, 1H), 2.62 (s, 3H), 2.44 (s, 3H).

### Step-c: Synthesis of (E)-6-Chloro-3-(3-(4-methoxyphenyl)acryloyl)-4-methylquinolin-2(1H)-one (1c)

To a stirred solution of 3-acetyl-6-chloro-4-methylquinolin-2(1H)-one (1.0 g, 4.20 mmol) in methanol (20 mL) was added anisaldehyde (0.63 g, 4.60 mmol) and the reaction mixture was cooled to 0° C. and then 25% aq. sodium hydroxide (20 mL) was added slowly in drop-wise manner for 10-15 min. The reaction mixture was allowed to stir at room temperature for 16 h and progress of the reaction was monitored by TLC. The reaction mixture was cooled to 0° C. and acidified with 6 N HCl (up to pH˜4) and extracted with ethyl acetate (3×50 mL). The combined organic layer was washed with brine (25 mL), dried over Na₂SO₄ and evaporated under reduced pressure to get the crude compound which was purified by flash column chromatography using 30-40% ethyl acetate in hexane as an eluent to afford the titled compound as off-white solid. Yield: 0.60 g (61%); ¹H-NMR (300 MHz, DMSO-d6): ⊐ 12.07 (bs, 1H), 7.85 (d, J=2.1 Hz, 1H), 7.70 (d, J=8.7 Hz, 2H), 7.62 (dd, J=6.3 Hz, 2.4 Hz, 1H), 7.44-7.37 (m, 2H), 6.98-6.89 (m, 3H), 3.79 (s, 3H), 2.30 (s, 3H). MS: m/z 354.1 [M+H]⁺.

### Step-d: 6-Chloro-3-(5-(4-methoxyphenyl)-1-propionyl-4,5-dihydro-1H-pyrazol-3-yl)-4-methylquinolin-2(1H)-one (1d)

To a stirred solution of (E)-6-chloro-3-(3-(4-methoxyphenyl)acryloyl)-4-methylquinolin-2(1H)-one (600 mg, 1.69 mmol) in propionic acid (6 mL) was added hydrazine hydrate (0.6 mL) and reaction mixture was refluxed for 3 h. Progress of the reaction was monitored by TLC. The pH of the reaction mixture was adjusted to 10 with saturated NaHCO₃ solution and extracted with ethyl acetate (2×50 mL). The combined organic layer was washed with water (25 mL), brine solution (25 mL), dried over Na₂SO₄, filtered and evaporated under reduced pressure to get the crude compound which was purified by prep. HPLC to afford the titled compound as an off-white solid. Yield: 80 mg (11%); ¹H-NMR (300 MHz, DMSO-d6): [symbol] 12.75 (bs, 1H), 7.94 (d, J=2.1 Hz, 1H), 7.58 (dd, J=6.6 Hz, 2.1 Hz, 1H), 7.35 (d, J=8.7 Hz, 1H), 7.30 (d, J=6.6 Hz, 2H), 6.90 (d, J=8.7 Hz, 2H), 5.53 (dd, J=4.5 Hz & 7.2 Hz, 1H), 3.80-3.76 (m, 4H), 3.21 (dd, J=4.5 Hz &14.1 Hz, 1H), 2.69 (q, J=7.2 Hz, 2H), 2.61 (s, 3H), 1.14 (t, J=7.5 Hz, 3H); LCMS: m/z 424.2 [M+H]⁺.

### Example-2: Synthesis of 6-Chloro-3-(1-(cyclopropanecarbonyl)-5-(4-methoxyphenyl)-4,5-dihydro-1H-pyrazol-3-yl)-4-methylquinolin-2(1H)-one

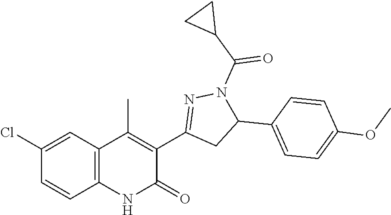 [SMILES: COc1ccc(C2CC(c3c(C)c4cc(Cl)ccc4[nH]c3=O)=NN2C(=O)C2CC2)cc1]

### Step-a: 6-Chloro-3-(5-(4-methoxyphenyl)-4,5-dihydro-1H-pyrazol-3-yl)-4-methylquinolin-2(1H)-one (2a)

To a solution of (E)-6-chloro-3-(3-(4-methoxyphenyl)acryloyl)-4-methylquinolin-2(1H)-one (200 mg, 0.56 mmol) in ethanol (10 mL) was added 99% hydrazine hydrate (0.63 g, 0.68 mmol) and refluxed for 16 h. Progress of the reaction was monitored by TLC. The reaction mixture was allowed to cool to room temperature and ethanol was evaporated under reduced pressure. The residue was diluted with water (25 mL) and solid obtained was filtered, washed with water and dried under vacuum to afford the titled compound. Yield: 110 mg (53%); ¹H-NMR (300 MHz, DMSO-d6): ⊐ 11.90 (bs, 1H), 7.80 (d, J=2.1 Hz 1H), 7.57-7.53 (m, 1H), 7.40-7.28 (m, 3H), 6.91 (d, J=10.5 Hz, 2H), 4.82-4.75 (m, 1H), 3.74 (s, 3H), 3.27-3.25 (m, 1H), 2.91-2.82 (m, 1H), [3H singlet merged in DMSO peak]. LC-MS: m/z 368.2 [M+H]⁺.

### Step-b: 6-Chloro-3-(1-(cyclopropanecarbonyl)-5-(4-methoxyphenyl)-4,5-dihydro-1H-pyrazol-3-yl)-4-methylquinolin-2(1H)-one (2)

To a stirred solution of (6-chloro-3-(5-(4-methoxyphenyl)-4,5-dihydro-1H-pyrazol-3-yl)-4-methylquinolin-2(1H)-one (110 mg, 0.29 mmol) in DCM (10 mL) at 0° C. was added pyridine (71 mg, 0.89 mmol), DMAP (5 mg) and cyclopropylcarbonylchloride (47 mg, 0.44 mmol). The reaction mixture was allowed to warm to room temperature and stirred for 2 h. Progress of the reaction was monitored by TLC. The reaction mixture was extracted with DCM (50 mL) and water (20 mL). The organic layer was washed brine (20 mL), dried over Na₂SO₄ and evaporated under reduced pressure to get crude compound which was purified by flash column chromatography using 50-60% ethyl acetate in hexane as an eluent to afford the titled compound as off-white solid. Yield: 0.030 g (23%); ¹H-NMR (300 MHz, DMSO-d6): ⊐ 12.45 (bs, 1H), 7.76 (d, J=2.1 Hz, 1H), 7.46 (dd, J=2.1 Hz & 6.6 Hz, 1H), 7.37 (d, J=8.4 Hz, 2H), 7.18 (d, J=8.7 Hz, 1H), 6.90 (d, J=8.7 Hz, 2H), 5.60 (dd, J=4.2 Hz & 7.8 Hz, 1H), 3.86-3.76 (m, 4H), 3.39 (dd, J=4.2 Hz & 14.1 Hz, 1H), 2.66 (s, 3H), 2.63-2.57 (m, 1H), 1.04-1.02 (m, 2H), 0.88-0.79 (m, 2H). LCMS: m/z 436.2 [M+H].

### Example-3: Synthesis of 6-chloro-3-(5-(4-methoxyphenyl)-1-(tetrahydro-2H-pyran-4-carbonyl)-4,5-dihydro-1H-pyrazol-3-yl)-4-methylquinolin-2(1H)-one

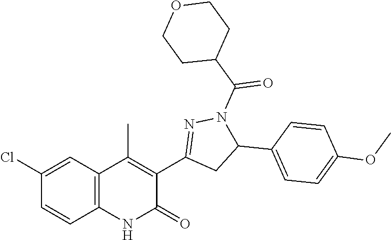 [SMILES: COc1ccc(C2CC(c3c(C)c4cc(Cl)ccc4[nH]c3=O)=NN2C(=O)C2CCOCC2)cc1]

The titled compound was synthesized using the same procedure which was followed for 6-Chloro-3-(1-(cyclopropanecarbonyl)-5-(4-methoxyphenyl)-4,5-dihydro-1H-pyrazol-3-yl)-4-methylquinolin-2(11H)-one (Example-2) using 6-Chloro-3-(5-(4-methoxyphenyl)-4,5-dihydro-1H-pyrazol-3-yl)-4-methylquinolin-2(1H)-one and tetrahydro-2H-pyran-4-carbonyl chloride as starting materials. Yield: 43%; ¹H-NMR (400 MHz, DMSO-d6): [symbol] 12.07 (bs, 1H), 7.89 (d, J=2.0 Hz, 1H), 7.62 (dd, J=2.4 Hz & 8.8 Hz, 1H), 7.36 (d, J=8.4 Hz, 1H), 7.21 (d, J=8.4 Hz, 2H), 6.91 (d, J=8.8 Hz, 2H), 5.47 (dd, J=4.4 Hz & 12.0 Hz, 1H), 3.86-3.84 (m, 2H), 3.81-3.76 (m, 1H), 3.74 (s, 3H), 3.38-3.36 (m, 2H), 3.27-3.25 (m, 1H), 3.08-3.02 (m, 1H), 1.63-1.59 (m, 4H); LCMS: m/z 480.20 [M+H]⁺. (3 protons merged in DMSO peak).

### Example-4: Synthesis of 6-chloro-3-(5-(4-methoxyphenyl)-1-(tetrahydrofuran-3-carbonyl)-4,5-dihydro-1H-pyrazol-3-yl)-4-methylquinolin-2(1H)-one

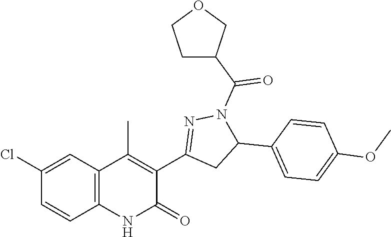 [SMILES: COc1ccc(C2CC(c3c(C)c4cc(Cl)ccc4[nH]c3=O)=NN2C(=O)C2CCOC2)cc1]

The titled compound was synthesized using the same procedure which was followed for 6-Chloro-3-(1-(cyclopropanecarbonyl)-5-(4-methoxyphenyl)-4,5-dihydro-1H-pyrazol-3-yl)-4-methylquinolin-2(11H)-one (Example-2) using 6-Chloro-3-(5-(4-methoxyphenyl)-4,5-dihydro-1H-pyrazol-3-yl)-4-methylquinolin-2(1H)-one and tetrahydrofuran-3-carbonyl chloride as starting materials. Yield: 47%. ¹H-NMR (400 MHz, DMSO-d6): ⊐ 12.07 (bs, 1H), 7.90 (d, J=2.0 Hz, 1H), 7.62 (dd, J=2.4 Hz & 8.8 Hz, 1H), 7.37 (d, J=8.8 Hz, 1H), 7.24 (d, J=8.4 Hz, 2H), 6.91 (d, J=8.4 Hz, 2H), 5.49-5.46 (m, 1H), 3.92-3.89 (m, 1H), 3.87-3.76 (m, 1H), 3.74 (s, 3H), 3.72-3.64 (m, 4H), 3.14-3.13 (m, 1H), 2.53 (s, 3H), 2.02 (t, J=8.4 Hz, 2H); LCMS: m/z 466.10 [M+H]⁺.

### Example-5: Synthesis of 6-Chloro-3-(5-(4-methoxyphenyl)-1-methyl-4,5-dihydro-1H-pyrazol-3-yl)-4-methylquinolin-2(1H)-one

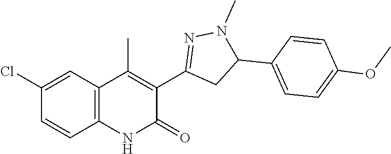 [SMILES: COc1ccc(C2CC(c3c(C)c4cc(Cl)ccc4[nH]c3=O)=NN2C)cc1]

### Step-a: 6-Chloro-3-(5-(4-methoxyphenyl)-1-methyl-4,5-dihydro-1H-pyrazol-3-yl)-4-methylquinolin-2(1H)-one

To a stirred solution of (E)-6-chloro-3-(3-(4-methoxyphenyl)acryloyl)-4-methylquinolin-2(1H)-one (100 mg 2.8 mmol) in ethanol (10 mL) was added triethyl amine (0.114 mL) and methylhydrazine sulfate (122 mg, 0.8 mmol). The reaction mixture was stirred at 90° C. for 16 h. Reaction progress was monitored by TLC. The volatiles were distilled off under reduced pressure and residue was diluted with dichloromethane (30 mL), washed with water (10 mL). The organic layer was dried over Na₂SO₄, filtered and evaporated under reduced pressure to get the crude compound which was purified by column chromatography using 70% ethyl acetate in hexane as an eluent to afford the titled compound as pale-yellow solid. Yield: 10 mg (10%); ¹H-NMR (300 MHz, CDCl₃): [symbol] [symbol] 10.47 (bs, 1H), 7.74 (d, J=2.1, 1H), 7.47-7.42 (m, 3H), 7.15 (d, J=8.7 Hz, 1H), 6.93 (d, J=8.7 Hz, 2H), 4.22 (dd, J=9.6 Hz & 14.7 Hz, 1H), 3.83 (s, 3H), 3.58 (dd, J=9.9 Hz & 16.2 Hz, 1H), 3.03 (dd, J=14.7 Hz & 16.2 Hz, 1H), 2.83 (s, 3H), 2.64 (s, 3H); LC-MS: m/z 382.2 [M+H]⁺.

### Example-6: Synthesis of 6-Chloro-4-methyl-3-(5-(1-methyl-1H-indol-6-yl)-1-propionyl-4,5-dihydro-1H-pyrazol-3-yl)quinolin-2(1H)-one

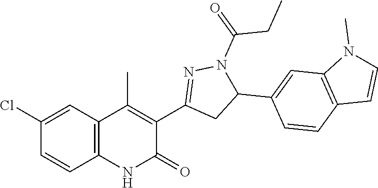 [SMILES: CCC(=O)N1N=C(c2c(C)c3cc(Cl)ccc3[nH]c2=O)CC1c1ccc2ccn(C)c2c1]

### Step-a: Synthesis of (E)-6-Chloro-4-methyl-3-(3-(1-methyl-1H-indol-3-yl)acryloyl)quinolin-2(1H)-one (6a)

The titled compound was synthesized using the same procedure which was followed for (E)-6-Chloro-3-(3-(4-methoxyphenyl)acryloyl)-4-methylquinolin-2(11H)-one (compound-1c) using 3-acetyl-6-chloro-4-methylquinolin-2(11H)-one and 1-methyl-1H-indole-6-carbaldehyde as starting materials. Yield: 38%; ¹H-NMR (300 MHz, CDCl₃): ⊐ 7.96 (s, 1H), 7.91 (d, J=7.8 Hz, 1H), 7.84 (t, J=2.1 Hz, 1H), 7.65-7.62 (m, 2H), 7.53 (d, J=7.8 Hz, 1H), 7.38 (d, J=9.3 Hz, 1H), 7.33-7.19 (m, 2H), 6.85 (d, J=16.2 Hz, 1H), 3.80 (s, 3H), 2.33 (s, 3H), 2.32 (s, 3H) (One exchangeable proton not observed in NMR).

### Step-b: 6-Chloro-4-methyl-3-(5-(1-methyl-1H-indol-6-yl)-1-propionyl-4,5-dihydro-1H-pyrazol-3-yl)quinolin-2(1H)-one (6)

The titled compound was synthesized using the same procedure which was followed for 6-Chloro-3-(5-(4-methoxyphenyl)-1-propionyl-4,5-dihydro-1H-pyrazol-3-yl)-4-methylquinolin-2(1H)-one (Example-1). Yield 20%; ¹H-NMR (300 MHz, CDCl₃): [symbol] ⊐ 12.87 (s, 1H), 7.74 (d, J=1.8 Hz, 1H), 7.63 (d, J=8.1 Hz, 1H), 7.40 (s, 1H), 7.26-7.16 (m, 2H), 7.05 (t, J=9.9 Hz, 2H), 6.49 (d, J=3.0 Hz, 1H), 5.76 (dd, J=3.9 Hz & 11.7 Hz, 1H), 3.87-3.77 (m, 1H), 3.75 (s, 3H), 3.51 (dd, J=4.2 Hz & 18 Hz, 1H), 2.76 (q, J=7.5 Hz, 2H), 2.65 (s, 3H), 1.17 (t, J=7.5 Hz, 3H); LCMS (m/z): 447.3 [M+H]⁺.

### Example-7: Synthesis of 6-chloro-3-(5-(4-methoxyphenyl)-1-propionyl-4,5-dihydro-1H-pyrazol-3-yl)-4-methylquinolin-2(1H)-one

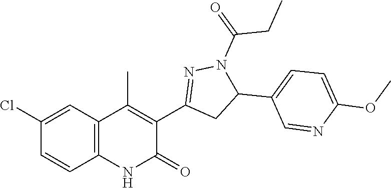 [SMILES: CCC(=O)N1N=C(c2c(C)c3cc(Cl)ccc3[nH]c2=O)CC1c1ccc(OC)nc1]

### Step-a: Synthesis of (E)-6-chloro-3-(3-(6-methoxypyridin-3-yl)acryloyl)-4-methylquinolin-2(1H)-one (7a)

The titled compound was synthesized using the same procedure which was followed for (E)-6-Chloro-3-(3-(4-methoxyphenyl)acryloyl)-4-methylquinolin-2(1H)-one (compound-1c) using 3-acetyl-6-chloro-4-methylquinolin-2(1H)-one and 6-methoxynicotinaldehyde as starting materials. Yield: 83%; LCMS: m/z 355.10 [M+H]⁺.

### Step-b: Synthesis of 6-chloro-3-(5-(6-methoxypyridin-3-yl)-1-propionyl-4,5-dihydro-1H-pyrazol-3-yl)-4-methylquinolin-2(1H)-one

To a stirred solution of (E)-6-chloro-3-(3-(6-methoxypyridin-3-yl)acryloyl)-4-methylquinolin-2(1H)-one (0.125 g, 0.35 mmol) in propionic acid (3 mL) was added hydrazine hydrate (0.12 mL) and the vial was placed in microwave and irradiated at 140° C. for 1 h. The reaction mixture was diluted with dichloromethane (30 mL), washed with saturated sodium bicarbonate solution (20 mL) and water (10 mL). The organic layer was dried over Na₂SO₄, filtered and evaporated under reduced pressure. The residue was purified by flash column chromatography using 2-5% methanol in dichloromethane as an eluent to afford the titled compound as off-white solid. Yield: 47 mg (30%); ¹H-NMR (400 MHz, DMSO-d6): ⊐ 12.01 (bs, 1H), 8.15 (s, 1H), 7.98 (s, 1H), 7.68 (d, J=8.4 Hz, 1H), 7.62 (d, J=8.4 Hz, 1H), 7.39 (d, J=8.8 Hz, 1H), 6.82 (d J=8.4, 1H), 5.51 (dd, J=2.0 Hz & 8.0 Hz, 1H), 3.84 (s, 3H), 3.76 (dd, J=6.0 Hz & 11.6 Hz, 1H), 3.18 (dd, J=4.0 Hz & 8.0 Hz, 1H), 2.62 (s, 3H), 2.59 (2H merged with DMSO peak), 1.01 (t, J=7.2, Hz, 3H); LCMS: m/z 425.20 [M+H]⁺.

### Example-8: Synthesis of N-(4-(3-(6-chloro-4-methyl-2-oxo-1,2-dihydroquinolin-3-yl)-1-propionyl-4,5-dihydro-1H-pyrazol-5-yl)phenyl)propionamide

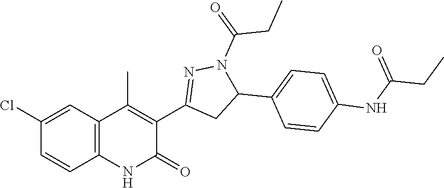 [SMILES: CCC(=O)Nc1ccc(C2CC(c3c(C)c4cc(Cl)ccc4[nH]c3=O)=NN2C(=O)CC)cc1]

### Step-a: Synthesis of (E)-N-(4-(3-(6-chloro-4-methyl-2-oxo-1,2-dihydroquinolin-3-yl)-3-oxoprop-1-en-1-yl)phenyl)acetamide (8a)

The titled compound was synthesized using the same procedure which was followed for (E)-6-Chloro-3-(3-(4-methoxyphenyl)acryloyl)-4-methylquinolin-2(1H)-one (compound-1c) using 3-acetyl-6-chloro-4-methylquinolin-2(1H)-one and N-(4-formylphenyl)acetamide as starting materials. Yield: 39%; LCMS: m/z 381.10 [M+H]⁺.

### Step-b: Synthesis of N-(4-(3-(6-chloro-4-methyl-2-oxo-1,2-dihydroquinolin-3-yl)-1-propionyl-4,5-dihydro-1H-pyrazol-5-yl)phenyl)propionamide

The titled compound was synthesized using the same procedure which was followed for 6-chloro-3-(5-(6-methoxypyridin-3-yl)-1-propionyl-4,5-dihydro-1H-pyrazol-3-yl)-4-methylquinolin-2(1H)-one (Example-7) using (E)-N-(4-(3-(6-chloro-4-methyl-2-oxo-1,2-dihydroquinolin-3-yl)-3-oxoprop-1-en-1-yl)phenyl)acetamide as starting material. Yield: 4%; ¹H-NMR (400 MHz, DMSO-d6): ⊐ 12.01 (bs, 1H), 9.86 (bs, 1H), 7.89 (s, 1H), 7.61 (d, J=8.8 Hz, 1H), 7.54 (d, J=8.4 Hz, 1H), 7.36 (d, J=8.4 Hz, 1H), 7.21 (d, J=8.0 Hz 2H), 5.47 (dd, J=8.4 Hz & 3.6 Hz, 1H), 3.78-3.72 (m, 1H), 3.11-3.05 (m, 1H), 2.62 (m, 4H), 2.29 (s, 3H) 1.07 (t, J=7.6 Hz, 3H), 1.03 (t, J=7.6 Hz, 3H); LCMS: m/z 465.10 [M+H]⁺.

### Example-9: Synthesis of: 3-(5-(4-Methoxyphenyl)-1-propionyl-4,5-dihydro-1H-pyrazol-3-yl)-4-methyl-1,5-naphthyridin-2(1H)-one

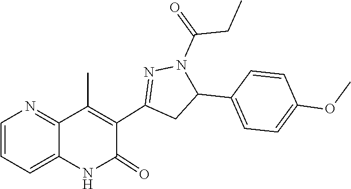 [SMILES: CCC(=O)N1N=C(c2c(C)c3ncccc3[nH]c2=O)CC1c1ccc(OC)cc1]

### Step-a: Synthesis of 3-Acetyl-4-methyl-1,5-naphthyridin-2(1H)-one (9a)

The titled compound was synthesized using the same procedure which was followed for 3-Acetyl-6-chloro-4-methylquinolin-2(1H)-one (compound-1b) using 1-(3-aminopyridin-2-yl)ethan-1-one and ethyl acetoacetate as starting materials. Yield: 38%; ¹H-NMR (300 MHz, CDCl₃): [symbol] 12.11 (s, 1H), 8.54 (d, J=3.3 Hz, 1H), 7.71 (d, J=7.8 Hz, 1H), 7.57 (dd, J=4.2 & 7.8 Hz, 1H), 7.57 (d, J=7.8 Hz, 1H), 2.47 (s, 3H), 2.45 (s, 3H).

### Step-b: Synthesis of (E)-3-(3-(4-Methoxyphenyl)acryloyl)-4-methyl-1,5-naphthyridin-2(1H)-one (9b)

The titled compound was synthesized using the same procedure which was followed for (E)-6-Chloro-3-(3-(4-methoxyphenyl)acryloyl)-4-methylquinolin-2(1H)-one (compound-1c) using 3-Acetyl-4-methyl-1,5-naphthyridin-2(11H)-one and 4-methoxybenzaldehyde as starting materials. Yield: 56%; ¹H-NMR (300 MHz, DMSO): [symbol] 12.03 (s, 1H), 8.56 (dd, J=1.5 & 4.2 Hz, 1H), 7.74-7.68 (m, 3H), 7.59 (q, J=4.2 Hz, 1H), 7.49 (d, J=16.2 Hz, 1H), 6.97 (t, J=6.6 Hz, 3H), 3.80 (s, 3H), 2.37 (s, 3H).

### Step-c: 3-(5-(4-Methoxyphenyl)-1-propionyl-4,5-dihydro-1H-pyrazol-3-yl)-4-methyl-1,5-naphthyridin-2(1H)-one (9)

The titled compound was synthesized using the same procedure which was followed for 6-Chloro-3-(5-(4-methoxyphenyl)-1-propionyl-4,5-dihydro-1H-pyrazol-3-yl)-4-methylquinolin-2(1H)-one (Example-1) using (E)-3-(3-(4-Methoxyphenyl)acryloyl)-4-methyl-1,5-naphthyridin-2(1H)-one as starting material. Yield: 21%; ¹H-NMR (300 MHz, CDCl₃): ⊐ 12.47 (bs, 1H), 8.65 (dd, J=1.5 Hz & 4.5 Hz, 1H), 7.55 (dd, J=1.5 Hz & 8.4 Hz, 1H), 7.43 (dd, J=4.2 Hz & 8.1 Hz, 1H), 7.38 (d, J=8.7 Hz, 2H), 6.91 (d, J=8.7 Hz, 1H), 5.60 (dd, J=3.9 Hz & 11.7 Hz, 1H), 3.87-3.77 (m, 4H), 3.41 (dd, J=4.2 Hz & 18.3 Hz, 1H), 2.81 (s, 3H), 2.75 (q, J=7.5 Hz, 2H) 1.18 (t, J=7.5 Hz, 3H); LCMS (m/z): 391 [M+H]⁺.

### Example-10: Synthesis of 4-Methyl-3-(5-(1-methyl-1H-indol-5-yl)-1-propionyl-4,5-dihydro-1H-pyrazol-3-yl)quinolin-2(1H)-one

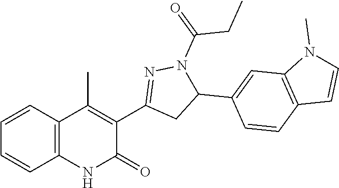 [SMILES: CCC(=O)N1N=C(c2c(C)c3ccccc3[nH]c2=O)CC1c1ccc2ccn(C)c2c1]

### Step-a: Synthesis of (E)-4-Methyl-3-(3-(1-methyl-1H-indol-5-yl)acryloyl)quinolin-2(1H)-one (10a)

The titled compound was synthesized using the same procedure which was followed for (E)-6-Chloro-3-(3-(4-methoxyphenyl)acryloyl)-4-methylquinolin-2(11H)-one (compound-1c) using 3-acetyl-4-methylquinolin-2(11H)-one and 1-methyl-1H-indole-5-carbaldehyde as starting materials. Yield: 59%; ¹H-NMR (300 MHz, CDCl₃): [symbol] 211.30 (bs, 1H), 7.77-7.75 (d, J=7.8 Hz, 1H) 7.63-7.58 (t, 2H), 7.47-7.30 (m, 4H), 7.26 (s, 1H), 7.13-7.12 (m, 2H), 6.49 (d, J=2.4 Hz, 1H), 3.77 (s, 3H), 2.48 (s, 3H).

### Step-b: Synthesis of 4-Methyl-3-(5-(1-methyl-1H-indol-5-yl)-1-propionyl-4,5-dihydro-1H-pyrazol-3-yl)quinolin-2(1H)-one

The titled compound was synthesized using the same procedure which was followed for 6-Chloro-3-(5-(4-methoxyphenyl)-1-propionyl-4,5-dihydro-1H-pyrazol-3-yl)-4-methylquinolin-2(1H)-one (Example-1) using (E)-4-Methyl-3-(3-(1-methyl-1H-indol-5-yl)acryloyl)quinolin-2(1H)-one as starting material. Yield: 17%; ¹H-NMR (300 MHz, CDCl₃): [symbol] 11.85 (s, 1H), 7.80 (d, J=7.8 Hz, 1H), 7.63 (d, J=8.4 Hz, 1H), 7.44 (bs, 1H), 7.36 (t, J=6.9 Hz, 1H), 7.28-7.19 (m, 3H), 7.03 (d, J=3.3 Hz, 1H), 6.47 (d, J=3.0 Hz, 1H), 5.75 (dd, J=4.5 & 11.7 Hz, 1H), 3.90 (dd, J=11.7 Hz & 18.3 Hz, 1H), 3.77 (s, 3H), 3.49 (dd, J=4.5 Hz & 18.0 Hz, 1H), 2.76 (q, J=7.5 Hz, 2H), 2.70 (s, 3H), 1.18 (t, J=7.5 Hz, 3H); LCMS (m/z): 413.3 [M+H]⁺.

### Example-11: Synthesis of 3-(5-(4-Methoxyphenyl)-1-propionyl-4,5-dihydro-1H-pyrazol-3-yl)-4-methylquinolin-2(1H)-one

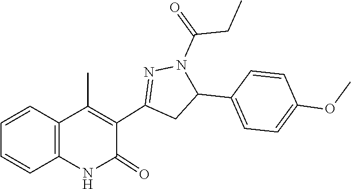 [SMILES: CCC(=O)N1N=C(c2c(C)c3ccccc3[nH]c2=O)CC1c1ccc(OC)cc1]

### Step-a: Synthesis of (E)-3-(3-(4-Methoxyphenyl)acryloyl)-4-methylquinolin-2(1H)-one (11a)

The titled compound was synthesized using the same procedure which was followed for (E)-6-Chloro-3-(3-(4-methoxyphenyl)acryloyl)-4-methylquinolin-2(1H)-one (compound-1c) using 3-acetyl-4-methylquinolin-2(1H)-one and 4-methoxybenzaldehyde as starting materials. Yield: 63%; ¹H-NMR (300 MHz, DMSO-d6): ⊐ 11.92 (s, 1H), 7.82 (d, J=7.2 Hz, 1H), 7.70 (d, J=8.7 Hz, 2H), 7.57 (t, J=7.8 Hz, 1H), 7.344-7.36 (m, 2H), 7.26 (t, J=7.8 Hz, 1H), 6.98-6.91 (m, 3H), 3.79 (s, 3H), 2.31 (s, 3H).

### Step-b: Synthesis of 3-(5-(4-Methoxyphenyl)-1-propionyl-4,5-dihydro-1H-pyrazol-3-yl)-4-methylquinolin-2(1H)-one

The titled compound was synthesized using the same procedure which was followed for 6-Chloro-3-(5-(4-methoxyphenyl)-1-propionyl-4,5-dihydro-1H-pyrazol-3-yl)-4-methylquinolin-2(1H)-one (Example-1) using (E)-3-(3-(4-Methoxyphenyl)acryloyl)-4-methylquinolin-2(1H)-one as starting material. Yield: 7%; ¹H-NMR (300 MHz, CDCl₃): ⊐ 13.07 (s, 1H), 7.81 (d, J=8.1 Hz, 1H), 7.53-7.43 (m, 3H), 7.32-7.29 (m, 2H), 6.92 (d, J=8.7 Hz, 2H), 5.61 (dd, J=3.9 Hz & 11.7 Hz, 1H), 3.83-3.73 (m, 4H), 3.48 (dd, J=3.9 Hz & 18.0 Hz, 1H), 2.74 (q, J=6.9 Hz, 2H), 2.66 (s, 3H), 1.18 (t, J=7.5 Hz, 3H); LCMS (m/z): 390 [M+H]⁺.

### Example-12: Synthesis of 4-Methyl-3-(5-(1-methyl-1H-indol-3-yl)-1-propionyl-4,5-dihydro-1H-pyrazol-3-yl)quinolin-2(1H)-one

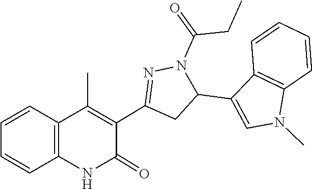 [SMILES: CCC(=O)N1N=C(c2c(C)c3ccccc3[nH]c2=O)CC1c1cn(C)c2ccccc12]

### Step-a: Synthesis of (E)-4-Methyl-3-(3-(1-methyl-1H-indol-3-yl)acryloyl)quinolin-2(1H)-one (12a)

The titled compound was synthesized using the same procedure which was followed for (E)-6-Chloro-3-(3-(4-methoxyphenyl)acryloyl)-4-methylquinolin-2(11H)-one (compound-1c) using 3-acetyl-4-methylquinolin-2(11H)-one and 1-methyl-1H-indole-3-carbaldehyde as starting materials. Yield: 50%; ¹H-NMR (300 MHz, DMSO-d₆): [symbol] 11.97 (bs, 1H), 7.82 (d, J=8.1 Hz, 1H), 7.56 (t, J=8.1 Hz, 1H), 7.33 (d, J=8.1 Hz, 1H), 7.25 (t, J=8.1 Hz, 1H), 2.45 (s, 3H), 2.33 (s, 3H).

### Step-b: 4-methyl-3-(5-(1-methyl-1H-indol-3-yl)-1-propionyl-4,5-dihydro-1H-pyrazol-3-yl)quinolin-2(1H)-one (12)

The titled compound was synthesized using the same procedure which was followed for 6-Chloro-3-(5-(4-methoxyphenyl)-1-propionyl-4,5-dihydro-1H-pyrazol-3-yl)-4-methylquinolin-2(1H)-one (Example-1) using (E)-4-Methyl-3-(3-(1-methyl-1H-indol-3-yl)acryloyl)quinolin-2(1H)-one as starting material. Yield: 92%; ¹H-NMR (300 MHz, CDCl₃): [symbol] 12.16 (s, 1H), 7.86 (d, J=8.4 Hz, 1H), 7.63 (d, J=7.8 Hz, 1H), 7.57 (t, J=7.5 Hz, 1H), 7.37 (t, J=9.0 Hz, 2H), 7.29-7.25 (m, 2H), 7.15 (t, J=7.5 Hz, 1H), 7.01 (t, J=7.5 Hz, 1H), 5.76 (dd, J=4.8 Hz & 12.3 Hz, 1H), 3.72 (bs, 4H) [signals merged with moisture of DMSO), 3.27 (dd, J=4.8 Hz & 18.9 Hz), 2.57 (bs, 5H), 0.99 (t, J=7.2 Hz, 3H); LCMS (m/z): 413 [M+H]⁺.

### Example-13: Synthesis of 3-(5-(4-Methoxyphenyl)-1-propionyl-4,5-dihydro-1H-pyrazol-3-yl)-4-methyl-1,8-naphthyridin-2(1H)-one

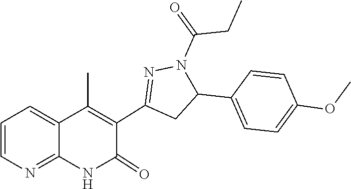 [SMILES: CCC(=O)N1N=C(c2c(C)c3cccnc3[nH]c2=O)CC1c1ccc(OC)cc1]

### Step-a: Synthesis of 3-Acetyl-4-methyl-1,8-naphthyridin-2(1H)-one (13a)

The titled compound was synthesized using the same procedure which was followed for 3-Acetyl-6-chloro-4-methylquinolin-2(1H)-one (compound-1b) using 1-(2-aminopyridin-3-yl) ethan-1-one and ethyl acetoacetate as starting materials. Yield: 23%; ¹H-NMR (300 MHz, DMSO-d6): [symbol] 12.35 (s, 1H), 8.56 (dd, J=1.5 Hz & 4.8 Hz, 1H), 8.27 (d, J=7.8 Hz, 1H), 7.31 (dd, J=4.8 Hz & 8.1 Hz, 1H), 2.45 (s, 3H), 2.33 (s, 3H).

### Step-b: Synthesis of (E)-3-(3-(4-Methoxyphenyl)acryloyl)-4-methyl-1,8-naphthyridin-2(1H)-one (13b)

The titled compound was synthesized using the same procedure which was followed for (E)-6-Chloro-3-(3-(4-methoxyphenyl)acryloyl)-4-methylquinolin-2(11H)-one (compound-1c) using 3-acetyl-4-methyl-1,8-naphthyridin-2(11H)-one and 4-methoxybenzaldehyde as starting materials. Yield: 55%; ¹H-NMR (300 MHz, DMSO-d6): [symbol] 12.29 (s, 1H), 8.58-8.56 (m, 1H), 8.26 (d, J=7.8 Hz, 1H), 7.69 (d, J=8.7 Hz, 2H), 7.46 (d, J=16.2 Hz, 1H), 7.35-7.31 (m, 1H), 6.98-6.90 (m, 3H), 3.80 (s, 3H), 2.31 (s, 3H).

### Step-c: Synthesis of 3-(5-(4-Methoxyphenyl)-1-propionyl-4,5-dihydro-1H-pyrazol-3-yl)-4-methyl-1,8-naphthyridin-2(1H)-one

The titled compound was synthesized using the same procedure which was followed for 6-Chloro-3-(5-(4-methoxyphenyl)-1-propionyl-4,5-dihydro-1H-pyrazol-3-yl)-4-methylquinolin-2(1H)-one (Example-1) using (E)-3-(3-(4-Methoxyphenyl)acryloyl)-4-methyl-1,8-naphthyridin-2(1H)-one as starting material. Yield: 9%; ¹H-NMR (300 MHz, CDCl₃): [symbol] 10.57 (s, 1H), 8.66 (dd, J=1.5 Hz & 4.5 Hz, 1H), 8.14 (dd, J=1.5 Hz & 8.1 Hz, 1H), 7.32-7.27 (m, 3H), 6.88 (d, J=8.7 Hz, 2H), 5.55 (dd J=4.8 Hz & 12.0 Hz, 1H), 3.92 (dd, J=12.0 Hz & 18.3 Hz, 1H), 3.79 (s, 3H), 3.28 (dd, J=4.5 Hz & 18.3 Hz, 1H), 2.71 (q, J=7.5 Hz, 2H), 2.66 (s, 3H) 1.16 (t, J=7.5 Hz, 3H); LCMS (m/z): 391.3 [M+H]⁺.

### Example-14: Synthesis of 6-(5-(4-Methoxyphenyl)-1-propionyl-4,5-dihydro-1H-pyrazol-3-yl)-5-methylpyrido-[2,3-d]pyrimidin-7(8H)-one

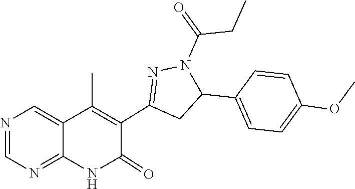 [SMILES: CCC(=O)N1N=C(c2c(C)c3cncnc3[nH]c2=O)CC1c1ccc(OC)cc1]

### Step-a: Synthesis of 5-Bromopyrimidin-4-amine (14a)

To a stirred solution of pyrimidin-4-amine (4.0 g, 42.5 mmol) in water (40 mL) was added calcium carbonate (2.10 g, 21 mmol) and followed by the dropwise addition of bromine (4 mL). The reaction mixture was heated at 60° C. for 2 h and the progress of the reaction was monitored by TLC. The reaction mixture was neutralised with aqueous ammonia (20 mL) and extracted with ethyl acetate (200 mL). The organic layer was washed with water (50 mL), brine solution (50 mL), dried over Na₂SO₄ and evaporated under reduced pressure to get the crude compound which was purified by column chromatography using 30% ethyl acetate in hexane as eluent to afford the titled compound. Yield: 2.0 g (28%). ¹H-NMR (300 MHz, CDCl₃): ⊐ 8.32 (s, 1H), 7.24 (S, 1H).

### Step-b: Synthesis of 1-(4-Aminopyrimidin-5-yl)ethan-1-one (14b)

To a stirred solution of 5-bromopyrimidin-4-amine (0.5 g, 2.8 mmol) in dioxane (5 mL) was added triethyl amine (0.853 mL, 5.78 mmol) and tributyl(1-ethoxyvinyl)stannane (1.35 g, 3.75 mmol). The reaction mixture was stirred at room temperature under nitrogen purging for 10 min. Pd(PPh₃)₂Cl₂ (0.039 g 0.56 mmol) was added to the reaction mixture and it was heated at 90° C. for overnight. Progress of the reaction was monitored by TLC. The reaction mixture was diluted with dichloromethane (30 mL), washed water (10 mL). The organic layer was dried over Na₂SO₄, filtered and evaporated under reduced pressure. The residue was purified by flash column chromatography using 5-10% ethyl acetate in hexane as eluent to afford the titled compound. Yield: 0.150 g (38%). ¹H-NMR (300 MHz, CDCl₃): [symbol] 8.84 (s, 1H), 8.60 (s, 1H), 5.60 (bs, 2H), 2.59 (s, 3H).

### Step-c: 6-Acetyl-5-methylpyrido[2,3-d]pyrimidin-7(8H)-one (14c)

To a stirred solution of 1-(4-aminopyrimidin-5-yl)ethan-1-one (0.4 g 2.9 mmol) in toluene (10 mL) were added cerium chloride (1.6 g, 4.2 mmol) and ethyl acetoacetate (0.549 g, 4.9 mmol). The reaction mixture was heated at 130° C. for 2 days and then another 2 h at 190° C. Reaction progress was monitored by TLC. The reaction mixture was diluted with dichloromethane (30 mL), washed with saturated NaHCO₃ solution (20 mL) and water (10 mL). The organic layer was dried over Na₂SO₄, filtered and evaporated under reduced pressure. The residue was purified by flash column chromatography using 30% ethyl acetate in hexane as eluent to afford the titled compound. Yield 0.3 g (51%). ¹H-NMR (300 MHz, DMSO-d₆): [symbol] 12.76 (bs, 1H), 9.20 (s, 1H), 9.01 (s, 1H), 2.45 (s, 3H), 2.38 (s, 3H).

### Step-d: (E)-6-(3-(4-methoxyphenyl) acryloyl)-5-methylpyrido[2,3-d]pyrimidin-7(8H)-one (14d)

The titled compound was synthesized using the same procedure which was followed for (E)-6-Chloro-3-(3-(4-methoxyphenyl)acryloyl)-4-methylquinolin-2(1H)-one (compound-1c) using 6-acetyl-5-methylpyrido[2,3-d]pyrimidin-7(8H)-one and 4-methoxybenzaldehyde as starting materials. Yield: 42%; ¹H-NMR (300 MHz, CDCl₃): ⊐ 12.68 (s, 1H), 9.19 (s, 1H), 9.02 (s, 1H), 7.70 (d, J=8.7 Hz, 2H), 7.51 (d, J=16.5 Hz, 1H), 6.98 (d, J=8.7 Hz, 2H), 6.91 (d, J=16.5 Hz, 1H), 3.80 (s, 3H), 2.34 (s, 3H).

### Step-e: 6-(5-(4-Methoxyphenyl)-1-propionyl-4,5-dihydro-1H-pyrazol-3-yl)-5-methylpyrido-[2,3-d]pyrimidin-7(8H)-one

The titled compound was synthesized using the same procedure which was followed for 6-Chloro-3-(5-(4-methoxyphenyl)-1-propionyl-4,5-dihydro-1H-pyrazol-3-yl)-4-methylquinolin-2(1H)-one (Example-1) using (E)-6-(3-(4-methoxyphenyl) acryloyl)-5-methylpyrido[2,3-d]pyrimidin-7(8H)-one as starting material. Yield: 27%; ¹H-NMR (300 MHz, CDCl₃): [symbol] 79.93 (bs, 1H), 9.12 (s, 1H), 9.07 (s, 1H), 7.27 (d, J=8.4 Hz, 2H), 6.88 (d, J=8.7 Hz, 2H), 5.55 (dd, J=4.5 & 12.0 Hz, 1H), 3.89 (dd, J=12.0 & 18.6 Hz, 1H), 3.78 (s, 3H), 3.27 (dd, J=4.5 Hz & 18.6 Hz, 1H), 2.74-2.71 (m, 5H), 1.16 (t, J=7.5 Hz, 3H), LCMS (m/z): 392.2 [M+H]⁺.

### Example-15: Synthesis of 6-bromo-3-(5-(4-methoxyphenyl)-1-propionyl-4,5-dihydro-1H-pyrazol-3-yl)-4-methylquinolin-2(1H)-one

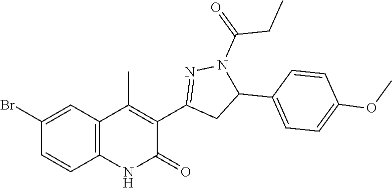 [SMILES: CCC(=O)N1N=C(c2c(C)c3cc(Br)ccc3[nH]c2=O)CC1c1ccc(OC)cc1]

### Step-a: Synthesis of 1-(2-amino-5-bromophenyl)ethan-1-one (15a)

The titled compound was synthesized using the same procedure which was followed for 1-(2-Amino-5-chlorophenyl)ethan-1-one (compound-1a) using 2-amino-5-bromobenzonitrile as starting material. Yield: 31%; LCMS: m/z 216.0 [M+2H]⁺.

### Step-b: Synthesis of 3-acetyl-6-bromo-4-methylquinolin-2(1H)-one (15b)

To a stirred solution of 1-(2-amino-5-bromophenyl)ethan-1-one (0.7 g 3.097 mmol) in DMF (10 mL) at ambient temperature was added ethyl acetoacetate (1.2 g, 9.29 mmol) and the reaction vial was placed in microwave and irradiated at 180° C. for 1.5 hours. Reaction mixture was brought to room temperature and diluted with water (30 mL) and extracted with ethyl acetate (2×75 mL). The combined organic layer was washed with brine (50 mL), dried over Na₂SO₄ and evaporated under reduced pressure to get crude compound which was purified by flash column chromatography using 40-50% ethyl acetate in hexane as eluent to afford the titled compound as a yellow solid. Yield: 0.65 g (75%); ¹H-NMR (400 MHz, DMSO-d6): ⊐ 11.12 (bs, 1H), 7.97 (d, J=2.0 Hz, 1H), 7.73 (dd, J=2.0 Hz & 8.8 Hz, 1H), 7.28 (d, J=8.8 Hz, 1H), 2.45 (s, 3H), 2.32 (s, 3H). LCMS: m/z 281.9 [M+2H]⁺.

### Step-c: Synthesis of (E)-6-bromo-3-(3-(4-methoxyphenyl)acryloyl)-4-methylquinolin-2(1H)-one (15c)

The titled compound was synthesized using the same procedure which was followed for (E)-6-Chloro-3-(3-(4-methoxyphenyl)acryloyl)-4-methylquinolin-2(1H)-one (compound-1c) using 1-(2-amino-5-bromophenyl)ethan-1-one and 4-methoxybenzaldehyde as starting materials. Yield: 98%; ¹H-NMR (400 MHz, DMSO-d6): ⊐ 12.05 (bs, 1H), 7.97 (d, J=2.4 Hz, 1H), 7.74 (d, J=2.0 Hz, 1H), 7.72 (d, J=2.0 Hz, 1H), 7.70 (d, J=8.8 Hz, 1H), 7.42 (d, J=16.0 Hz, 1H), 7.31 (d, J=16.0 Hz, 1H), 6.94 (d, J=8.8 Hz, 2H), 6.24 (d, J=16.4 Hz, 1H), 3.80 (s, 3H), 2.32 (s, 3H); LCMS: m/z 400.0 [M+2H]⁺.

### Step-d: Synthesis of 6-bromo-3-(5-(4-methoxyphenyl)-1-propionyl-4,5-dihydro-1H-pyrazol-3-yl)-4-methylquinolin-2(1H)-one

The titled compound was synthesized using the same procedure which was followed for 6-chloro-3-(5-(4-methoxyphenyl)-1-propionyl-4,5-dihydro-1H-pyrazol-3-yl)-4-methylquinolin-2(1H)-one (Example-7) using (E)-6-bromo-3-(3-(4-methoxyphenyl)acryloyl)-4-methylquinolin-2(1H)-one as starting material. Yield: 35%; ¹H-NMR (400 MHz, DMSO-d6): [symbol] 12.06 (bs, 1H), 8.00 (d, J=2.0 Hz, 1H), 7.72 (dd, J₁=2.4 Hz & 8.8 Hz, 1H), 7.30 (d, J=8.8 Hz, 1H), 7.23 (d, J=8.8 Hz, 2H), 6.90 (d, J=8.8 Hz, 2H), 5.46 (dd, J₁=4.0 Hz & 11.2 Hz, 1H), 3.79-3.76, (m, 1H), 3.74 (s, 3H), 3.015 (dd, J₁=4.4 Hz & 18.4 Hz, 1H), 2.60 (m, 2H), 2.52 (s, 3H), 1.01 (t, J=7.2, Hz, 3H); LCMS: m/z 470.1 [M+H]⁺.

### Example-16: 6-chloro-3-(5-(4-methoxyphenyl)-1-propionyl-4,5-dihydro-1H-pyrazol-3-yl)-4-methyl-1,5-naphthyridin-2(1H)-one

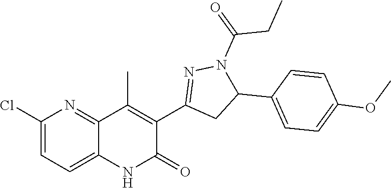 [SMILES: CCC(=O)N1N=C(c2c(C)c3nc(Cl)ccc3[nH]c2=O)CC1c1ccc(OC)cc1]

### Step-a: Synthesis of 3-amino-6-chloropicolinonitrile (16a)

To a stirred solution of 6-chloro-3-nitropicolinonitrile (0.5 g, 2.72 mmol) in a mixture of ethanol and conc. HCl (3:1, 8 mL) was added iron powder (0.52 g, 9.52 mmol) and the reaction mixture was stirred at 90° C. for 30 min. Progress of the reaction was monitored by TLC. The reaction mixture was poured over ice water and basified with aqueous ammonia and filtered through celite. The filtrate was extracted with diethyl ether (3×100 mL). Combined organic layer was dried over Na₂SO₄ and evaporated under reduced pressure to afford the titled compound as yellow solid. Yield: 0.35 g (77%). ¹H-NMR (400 MHz, DMSO-d6): [symbol] 7.42 (d, J=9.2 Hz, 1H), 7.28 (d, J=8.8 Hz, 1H), 6.58 (bs, 2H); LCMS: m/z 156.9 [M+H]⁺.

### Step-b: Synthesis of 1-(3-amino-6-chloropyridin-2-yl)ethan-1-one (16b)

The titled compound was synthesized using the same procedure which was followed for 1-(2-Amino-5-chlorophenyl)ethan-1-one (compound-1a) using 3-amino-6-chloropicolinonitrile as starting material. Yield: 77%; ¹H-NMR (400 MHz, CDCl₃): ⊐ 7.23 (d, J=8.8 Hz, 1H), 7.01 (d, J=8.8 Hz, 1H), 5.48 (bs, 2H), 2.66 (s, 3H); LCMS: m/z 171.0 [M+H]⁺.

### Step-c: Synthesis of 3-acetyl-6-chloro-4-methyl-1,5-naphthyridin-2(1H)-one (16c)

The titled compound was synthesized using the same procedure which was followed for 3-acetyl-6-bromo-4-methylquinolin-2(1H)-one (compound-15b) using 1-(3-amino-6-chloropyridin-2-yl)ethan-1-one as starting material. Yield: 21%; ¹H-NMR (400 MHz, DMSO-d6): ⊐ 12.27 (bs, 1H), 7.74 (d, J=8.4 Hz, 1H), 7.67 (d, J=8.4 Hz, 1H), 2.47 (s, 3H), 2.34 (s, 3H). LCMS: m/z 237.0 [M+H]⁺.

### Step-d: Synthesis of (E)-6-chloro-3-(3-(4-methoxyphenyl)acryloyl)-4-methyl-1,5-naphthyridin-2(1H)-one (16d)

The titled compound was synthesized using the same procedure which was followed for (E)-6-Chloro-3-(3-(4-methoxyphenyl)acryloyl)-4-methylquinolin-2(1H)-one (compound-1c) using 3-acetyl-6-chloro-4-methyl-1,5-naphthyridin-2(1H)-one and 4-methoxybenzaldehyde as starting materials. Yield: 66%; LCMS: m/z 355.1 [M+H]⁺.

### Step-e: Synthesis of 6-chloro-3-(5-(4-methoxyphenyl)-1-propionyl-4,5-dihydro-1H-pyrazol-3-yl)-4-methyl-1,5-naphthyridin-2(1H)-one

The titled compound was synthesized using the same procedure which was followed for 6-chloro-3-(5-(4-methoxyphenyl)-1-propionyl-4,5-dihydro-1H-pyrazol-3-yl)-4-methylquinolin-2(1H)-one (Example-7) using (E)-6-chloro-3-(3-(4-methoxyphenyl)acryloyl)-4-methyl-1,5-naphthyridin-2(1H)-one as starting material. Yield: 46%; ¹H-NMR (400 MHz, DMSO-d6): ⊐ 12.22 (bs, 1H), 7.75 (d, J=8.4 Hz, 1H), 7.66 (d, J=8.4 Hz, 1H), 7.21 (d, J=8.4 Hz, 2H), 6.91 (d, J=8.4 Hz, 2H), 5.46 (dd, J=8.4 Hz & 4.0 Hz, 1H), 3.80 (m, 1H), 3.74 (s, 3H), 3.17-3.11 (m, 1H), 2.64 (q, J=7.6 Hz, 2H), 2.57 (s, 3H), 1.06 (t, J=9.2 Hz, 3H), LCMS: m/z 425.3 [M+H]⁺.

### Example-17: Synthesis of 6-Chloro-3-(5-(4-methoxyphenyl)-1-propionyl-1H-pyrazol-3-yl)quinolin-2(1H)-one

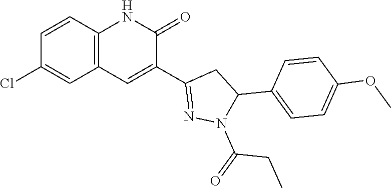 [SMILES: CCC(=O)N1N=C(c2cc3cc(Cl)ccc3[nH]c2=O)CC1c1ccc(OC)cc1]

### Step-a: Synthesis 3-Acetyl-6-chloroquinolin-2(1H)-one (17a)

To a stirred solution of 2-amino-5-chlorobenzaldehyde (3.0 g 1.9 mmol) in xylene (20 mL) was added 2,2,6-trimethyl-4H-1,3-dioxin-4-one (1.91 g 1.3 mmol) and the reaction mixture was heated at 190° C. for 2 h. Progress of the reaction was monitored by TLC. The reaction mixture was cooled to room temperature and filtered. The solid obtained was washed with xylene and dried under vacuum to afford the titled compound. Yield 1.2 g (28%); ¹H-NMR (300 MHz, CDCl₃): ⊐ 12.23 (s, 1H), 8.43 (s, 1H), 8.01 (d, J=2.1 Hz, 1H), 7.65 (dd, J=2.4 Hz & 8.7 Hz, 1H), 7.35 (d, J=8.7 Hz, 1H), 2.61 (s, 3H).

### Step-b: Synthesis of (E)-6-Chloro-3-(3-(4-methoxyphenyl)acryloyl)quinolin-2(1H)-one (17b)

The titled compound was synthesized using the same procedure which was followed for (E)-6-Chloro-3-(3-(4-methoxyphenyl)acryloyl)-4-methylquinolin-2(1H)-one (compound-1c) using 3-Acetyl-6-chloroquinolin-2(1H)-one and 4-methoxybenzaldehyde as starting materials. Yield: 65%. ¹H-NMR (300 MHz, CDCl₃): [symbol] 8.38 (s, 1H), 7.97 (d, J=2.1 Hz, 1H), 7.70 (d, J=8.7 Hz, 2H), 7.64-7.61 (m, 3H), 7.37 (d, J=8.7 Hz, 1H), 7.01 (d, J=8.7 Hz, 2H), 3.81 (s, 3H), [NH proton is not visible in NMR].

### Step-c: Synthesis of 6-Chloro-3-(5-(4-methoxyphenyl)-1-propionyl-4,5-dihydro-1H-pyrazol-3-yl)quinolin-2(1H)-one

The titled compound was synthesized using the same procedure which was followed for 6-Chloro-3-(5-(4-methoxyphenyl)-1-propionyl-4,5-dihydro-1H-pyrazol-3-yl)-4-methylquinolin-2(1H)-one (Example-1) using (E)-6-Chloro-3-(3-(4-methoxyphenyl)acryloyl)quinolin-2(1H)-one as starting material. Yield: 57%; 1H-NMR (300 MHz, CDCl₃): [symbol] 12.16 (s, 1H), 8.49 (s, 1H), 7.96 (d, J=2.4 Hz, 1H), 7.58 (dd, J=2.4 Hz & 8.7 Hz, 1H), 7.32 (d, J=8.7 Hz, 1H), 7.11 (d, J=8.7 Hz, 2H), 6.88 (d, J=8.7 Hz, 2H), 5.45 (dd, J=4.5 Hz & 12.0 Hz, 1H), 3.88 (dd, J=12.0 Hz & 18.9 Hz, 1H), 3.72 (s, 3H), 3.27 (dd, J=4.5 Hz & 18.9 Hz, 1H), 2.72 (q, J=7.5 Hz, 2H), 1.06 (t, J=7.5 Hz, 3H); LCMS (m/z): 410 [M+H]⁺.

### Example-18: Synthesis of 6-chloro-3-(5-(4-methoxyphenyl)-1-propionyl-4,5-dihydro-1H-pyrazol-3-yl)-1,4-dimethylquinolin-2(1H)-one

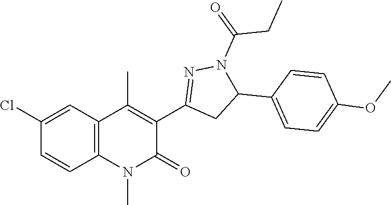 [SMILES: CCC(=O)N1N=C(c2c(C)c3cc(Cl)ccc3n(C)c2=O)CC1c1ccc(OC)cc1]

### Step-a: 6-chloro-3-(5-(4-methoxyphenyl)-1-propionyl-4,5-dihydro-1H-pyrazol-3-yl)-1,4-dimethylquinolin-2(1H)-one

To a stirred solution of 6-chloro-3-(5-(4-methoxyphenyl)-1-propionyl-4,5-dihydro-1H-pyrazol-3-yl)-4-methylquinolin-2(1H)-one (40 mg, 0.094 mmol) in DMF (3 mL) at 0° C. was added K₂CO₃ (39 mg, 0.28 mmol) and followed by addition of methyl iodide (14 mg, 0.104 mmol). The reaction mixture was stirred at 60° C. for 3 h. Reaction progress was monitored by TLC. The reaction mixture was diluted with EtOAC (30 mL), water (10 mL) and extracted. The organic layer was washed with brine (10 mL), dried over Na₂SO₄, filtered and evaporated under reduced pressure. The residue was purified by flash column chromatography using 2-5% methanol in dichloromethane as eluent to afford the titled compound as off-white solid. Yield: 19 mg (46%); ¹H-NMR (400 MHz, DMSO-d6): [symbol] 7.96 (d, J=2.4 Hz, 1H), 7.74 (dd, J=2.0 Hz & 9.2 Hz, 1H), 7.63 (d, J=9.2 Hz, 1H), 7.27 (d, J=8.4 Hz, 2H), 6.91 (d, J=8.4 Hz, 2H), 5.49-5.45 (dd, J=4.0 & 7.6 Hz, 1H), 3.74 (s, 3H), 3.69 (t, J=6.4 Hz, 1H), 3.64 (s, 3H), 3.11-3.06 (m, 1H), 2.58 (q, J=7.4 Hz, 2H), 2.53 (s, 3H), 1.01 (t, J=7.2 Hz, 3H); LCMS: m/z 438.1 [M+H]⁺.

### Example-19: 4-(5-(4-methoxyphenyl)-1-propionyl-4,5-dihydro-1H-pyrazol-3-yl)isoquinolin-3(2H)-one

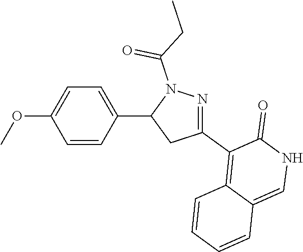 [SMILES: CCC(=O)N1N=C(c2c(=O)[nH]cc3ccccc23)CC1c1ccc(OC)cc1]

### Step-a: Synthesis of 4-bromoisoquinolin-3(2H)-one (19a)

To a stirred solution of isoquinolin-3(2H)-one (0.3 g, 2.06 mmol) in DMF (10 mL) at 0° C. was added NBS (0.404 g, 2.27 mmol) portion wise over a period of 5 min. The reaction mixture was stirred at room temperature for 16 h. Progress of the reaction was monitored by TLC. The reaction mixture was poured over ice water. The solid formed was filtered, washed with water and dried under vacuum to afford the titled compound. Yield: 0.275 g (50%). ¹H-NMR (400 MHz, DMSO-d6): ⊐ 18.91 (s, 1H), 8.01 (d, J=8.0 Hz, 1H), 7.87 (d, J=8.8 Hz, 1H), 7.74 (t, J=8.0 Hz, 1H), 7.41 (t, J=7.2 Hz, 1H), [NH proton is not visible in NMR]; LCMS: m/z 225.9 [M+2H]⁺.

### Step-b: Synthesis of 4-acetylisoquinolin-3(2H)-one (19b)

The titled compound was synthesized using the same procedure which was followed for 1-(4-Aminopyrimidin-5-yl)ethan-1-one (compound-14b) using 4-bromoisoquinolin-3(2H)-one as starting material. Yield: 38%; LCMS: m/z 188.0 [M+H]⁺.

### Step-c: Synthesis of (E)-4-(3-(4-methoxyphenyl)acryloyl)isoquinolin-3(2H)-one (19c)

The titled compound was synthesized using the same procedure which was followed for (E)-6-Chloro-3-(3-(4-methoxyphenyl)acryloyl)-4-methylquinolin-2(1H)-one (compound-1c) using 4-acetylisoquinolin-3(2H)-one and 4-methoxybenzaldehyde as starting materials. Yield: 51%; LCMS: m/z 306.1 [M+H]⁺.

### Step-d: Synthesis of 4-(5-(4-methoxyphenyl)-1-propionyl-4,5-dihydro-1H-pyrazol-3-yl)isoquinolin-3(2H)-one

The titled compound was synthesized using the same procedure which was followed for 6-chloro-3-(5-(4-methoxyphenyl)-1-propionyl-4,5-dihydro-1H-pyrazol-3-yl)-4-methylquinolin-2(1H)-one (Example-7) using (E)-4-(3-(4-methoxyphenyl)acryloyl)isoquinolin-3(2H)-one as starting material. Yield: 29%; ¹H-NMR (400 MHz, DMSO-d6): ⊐ 8.92 (s, 1H), 8.38 (d, J=9.2 Hz, 1H), 7.96 (d, J=8.4 Hz, 1H), 7.66 (t, J=7.2 Hz, 1H), 7.31 (t, J=7.2 Hz, 1H), 7.19 (d, J=8.8 Hz, 2H), 6.89 (d, J=8.8 Hz, 2H), 5.46 (dd, J=4.0 Hz & 12.0 Hz, 1H), 3.95-3.98, (m, 1H), 3.72 (s, 3H), 3.27-3.26 (m, 1H), 2.69-2.66 (m, 2H), 2.57 (s, 3H), 1.06 (t, J=7.2 Hz, 3H); LCMS: m/z 376.2 [M+H]⁺.

### Example-20: Synthesis of 5-(5-(4-Methoxyphenyl)-1-propionyl-4,5-dihydro-1H-pyrazol-3-yl)-4-methylthieno-[2,3-b]pyridin-6(7H)-one

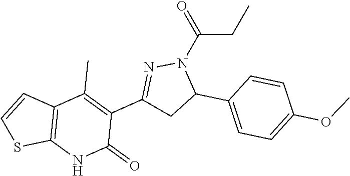 [SMILES: CCC(=O)N1N=C(c2c(C)c3ccsc3[nH]c2=O)CC1c1ccc(OC)cc1]

### Step-a: Synthesis of (N-(3-acetylthiophen-2-yl)-3-oxobutanamide (20a)

To a stirred solution of 1-(2-aminothiophen-3-yl)ethan-1-one (3.0 g 21.24 mmol) in xylene (20 mL) was added 2,2,6-trimethyl-4H-1,3-dioxin-4-one (3.62 g 25.48 mmol) and the reaction mixture was heated at 140° C. for 3 h. Progress of the reaction was monitored by TLC. The reaction mixture was cooled to room temperature and diluted with water (50 mL) and extracted with ethyl acetate (2×100 mL). The combined organic layer was washed with brine (50 mL), dried over Na₂SO₄, filtered and evaporated under reduced pressure to get the crude compound which was purified by flash column chromatography using 20-30% EtOAc in hexane as eluent to afford the titled compound as a colourless liquid. Yield: 4.5 g (93%); ¹H-NMR (400 MHz, DMSO-d6): [symbol] 12.17 (bs, 1H), 7.40 (d, J=6.0 Hz, 1H), 7.01 (d, J=5.6 Hz, 1H), 3.91 (s, 3H), 2.49 (s, 3H), 2.21 (s, 3H); LCMS (m/z): 223.9 [M−H]⁺.

### Step-b: Synthesis of 5-acetyl-4-methylthieno[2,3-b]pyridin-6(7H)-one (25b)

A mixture of (N-(3-acetylthiophen-2-yl)-3-oxobutanamide (4.5 g, 19.97 mmol) and cerium chloride heptahydrate (2.22 g, 5.99 mmol) was heated at 150° C. for 30 mins. Progress of the reaction was monitored by TLC. The reaction mixture was cooled to room temperature and diluted with ethyl acetate (200 mL), water (50 mL) and extracted. The organic layer was washed with brine (50 mL), dried over sodium sulfate, filtered and evaporated under reduced pressure. The residue was diluted with DCM (200 mL) and solid obtained was filtered and dried under vacuum to afford the titled compound as off-white solid. Yield: 1.6 g (38%); ¹H-NMR (400 MHz, DMSO-d6): ⊐ [symbol] 12.50 (bs, 1H), 7.32 (s, 2H), 2.46 (s, 3H), 2.35 (s, 3H); LCMS (m/z): 208.1 [M−H]⁺.

### Step-c: Synthesis of (E)-5-(3-(4-methoxyphenyl)acryloyl)-4-methylthieno[2,3-b]pyridin-6(7H)-one: (20c)

The titled compound was synthesized using the same procedure which was followed for (E)-6-Chloro-3-(3-(4-methoxyphenyl)acryloyl)-4-methylquinolin-2(1H)-one (compound-1c) using 5-acetyl-4-methylthieno[2,3-b]pyridin-6(7H)-one and 4-methoxybenzaldehyde as starting materials. Yield: 95%; LCMS (m/z): 326.2 [M−H]⁺.

### Step-d: Synthesis of 5-(5-(4-methoxyphenyl)-1-propionyl-4,5-dihydro-1H-pyrazol-3-yl)-4-methylthieno[2,3 b]pyridin-6(7H)-one: (20)

To a stirred solution of (E)-5-(3-(4-methoxyphenyl)acryloyl)-4-methylthieno[2,3-b]pyridin-6(7H)-one (7.5 g, 23.0 mmol) in propionic acid (75 mL) was added hydrazine hydrate (7.5 mL) and the reaction mixture was refluxed for 5 h. Progress of the mixture was monitored by TLC. The reaction mixture was allowed to cool to room temperature and quenched with saturated sodium bicarbonate solution (100 mL) and extracted with ethyl acetate (3×250 mL). The combined organic layer was washed with water (100 mL), brine (100 mL), dried over anhydrous sodium sulfate and evaporated under reduced pressure. The residue was purified by flash column chromatography using 2 to 2.5% methanol in DCM as eluent to afford the titled compound as off white solid. Yield: 5.0 g (54%); ¹H-NMR (400 MHz, DMSO-d6): [symbol] 12.45 (bs, 1H), 7.35-7.31 (m, 2H), 7.20 (d, J=8.8 Hz, 2H), 6.89 (d, J=8.8, 7.5 Hz, 2H), 5.43 (dd, J=4.4 Hz &12.0 Hz, 1H), 3.78-3.75 (m, 1H), 3.73 (s, 3H), 3.14 (dd, J=4.4 Hz &18.0 Hz, 1H), 2.62 (q, J=7.6 Hz, 2H), 2.54 (s, 3H), 1.03 (t, J=7.6 Hz, 3H). LCMS: m/z 396.2 [M+H]⁺.

### Chiral Purification of Compound 20.

Step-e: 20(A): HPLC using a chiral pack IA 5 uM, 4.6*250 mm, IPA/DCM (90/10) eluent at flow rate of 0.7 mL/min at 20° C. The isolated enantiomer at 14.21 min.

Step-f: 20 (B): HPLC using a chiral pack IA 5 uM, 4.6*250 mm, IPA/DCM (90/10) eluent at flow rate of 0.7 mL/min at 20° C. The isolated enantiomer at 19.36 min.

### Example-21: Synthesis of 5-(5-(6-methoxypyridin-3-yl)-1-propionyl-4,5-dihydro-1H-pyrazol-3-yl)-4-methylthieno[2,3-b]pyridin-6(7H)-one

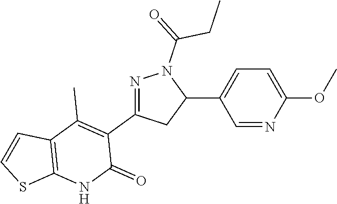 [SMILES: CCC(=O)N1N=C(c2c(C)c3ccsc3[nH]c2=O)CC1c1ccc(OC)nc1]

### Step-a: Synthesis of (E)-5-(3-(6-methoxypyridin-3-yl)acryloyl)-4-methylthieno[2,3-b]pyridin-6(7H)-one (21a)

The titled compound was synthesized using the same procedure which was followed for (E)-6-Chloro-3-(3-(4-methoxyphenyl)acryloyl)-4-methylquinolin-2(1H)-one (compound-1c) using 5-acetyl-4-methylthieno[2,3-b]pyridin-6(7H)-one and 6-methoxynicotinaldehyde as starting materials. Yield: 92%; LCMS: m/z 327.1 [M+H]⁺.

### Step-b: Synthesis of 5-(5-(6-methoxypyridin-3-yl)-1-propionyl-4,5-dihydro-1H-pyrazol-3-yl)-4-methylthieno[2,3-b]pyridin-6(7H)-one

The titled compound was synthesized using the same procedure which was followed for 6-chloro-3-(5-(4-methoxyphenyl)-1-propionyl-4,5-dihydro-1H-pyrazol-3-yl)-4-methylquinolin-2(1H)-one (Example-7) using (E)-5-(3-(6-methoxypyridin-3-yl)acryloyl)-4-methylthieno[2,3-b]pyridin-6(7H)-one as starting material. Yield: 35%; ¹H-NMR (400 MHz, DMSO-d6): [symbol] 8.35 (s, 1H), 7.85 (d, J=7.6, 1H), 7.19 (d, J=6.0 Hz, 1H), 7.06 (d, J=4.0 Hz, 1H), 6.85 (d, J=8.4 Hz, 1H), 5.57 (d J=8.8, Hz, 1H), 4.05 (s, 3H), 3.88-3.60 (m, 1H), 3.51-3.46 (m, 1H), 2.73 (q, J=7.6 Hz, 2H), 2.69 (s, 3H), 1.15 (t, J=7.2, Hz, 3H), [NH proton is not visible in NMR]; LCMS: m/z 397.1 [M+H]⁺.

### Example-22: Synthesis of 5-(5-(4-methoxyphenyl)-1-propionyl-4,5-dihydro-1H-pyrazol-3-yl)thieno[2,3-b]pyridin-6(7H)-one

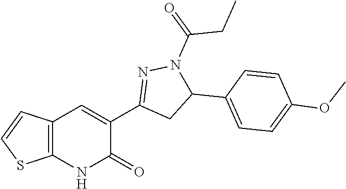 [SMILES: CCC(=O)N1N=C(c2cc3ccsc3[nH]c2=O)CC1c1ccc(OC)cc1]

### Step-a: Synthesis of 3-(dichloromethyl)-2-nitrothiophene (22a)

To a stirred solution of potassium tert-butoxide (23 mL, 23.2 mmol, 1M in THF) in DMF (15 mL) at −78° C. was added 2-nitrothiophene (1.0 g, 7.74 mmol dissolved in 5 mL of DMF) and chloroform (1.3 mL) and the reaction mixture was stirred for 5 min. The reaction mixture was quenched with methanol (2 mL), acetic acid (2 mL), diluted with water (20 mL) and extracted with ethyl acetate (3×20 mL). The combined organic layer was washed with brine (30 mL), dried over anhydrous sodium sulfate and evaporated under reduced pressure to get the crude compound which was purified by flash column chromatography using 3-5% ethyl acetate in hexane as eluent to afford the titled compound as pale yellow solid. Yield: 2.5 g (76%); 1H NMR (400 MHz, DMSO-d6): [symbol] 7.64 (s, 1H), 7.54-7.558 (m, 2H).

### Step-b: Synthesis of 2-nitrothiophene-3-carbaldehyde (22b)

A solution of 3-(dichloromethyl)-2-nitrothiophene (1.0 g, 4.71 mmol) in formic acid was heated at 100° C. for 48 h. Progress of the reaction was monitored by TLC. Upon completion, the reaction mixture was cooled to room temperature, concentrated under reduced pressure. The residue was diluted with aq. sodium bicarbonate solution (30 mL) and extracted with ethyl acetate (3×25 mL). The combined organic layer was washed with brine solution (15 mL), dried over anhydrous sodium sulfate, filtered and evaporated under reduced pressure to get the crude compound which was purified by flash column chromatography using 3-5% ethyl acetate in hexane as eluent to afford the titled compound as brown solid. Yield: 1.1 g (34%); ¹H NMR (400 MHz, DMSO-d6): [symbol] 10.61 (s, 1H), 7.54-7.48 (m, 2H).

### Step-c: Synthesis of 2-aminothiophene-3-carbaldehyde (22c)

To a stirred solution of 2-nitrothiophene-3-carbaldehyde (1.05 g, 6.68 mmol) in ethanol (13 mL) and water (2 mL) was added iron powder (1.87 g, 33.40 mmol) and ammonium chloride (3.67 g, 66.81 mmol). The reaction mixture was heated at 90° C. for 1 h. Reaction progress was monitored by TLC. The reaction mixture was cooled to RT, filtered through celite bed, and the celite bed was washed with methanol (30 mL). Combined filtrate was evaporated under reduce pressure. The residue was neutralized with saturated sodium bicarbonate solution (20 mL) and extracted with ethyl acetate (3×10 mL). The combined organic layer was washed with brine solution (10 mL), dried over anhydrous sodium sulfate and concentrated under reduced pressure to afford the titled compound as brown solid. Yield: 500 mg (58%); LCMS: m/z 128.0 [M+H]⁺.

### Step-d: Synthesis of 5-acetylthieno[2,3-b]pyridin-6(7H)-one (22d)

To a stirred solution of 2-aminothiophene-3-carbaldehyde (500 mg, 3.93 mmol) in DMF (8 mL) was added ethyl acetoacetate (0.75 mL, 5.89 mmol) and molecular sieves (200 mg). The reaction vial was placed in microwave reactor and irradiated at 180° C. for 2 h. The reaction mixture was cooled to room temperature and quenched with brine (10 mL) and then extracted with ethyl acetate (3×10 mL). The combined organic layer was washed with brine (10 mL), dried over Na₂SO₄ evaporated under reduced pressure to get the crude compound which was purified by flash chromatography using 20-30% ethyl acetate in hexane as eluent to afford the titled compound as a brown solid. Yield: 150 mg (20%); LCMS: m/z 193.9 [M+H]⁺.

### Step-e: Synthesis of (E)-5-(3-(4-methoxyphenyl)acryloyl)thieno[2,3-b]pyridin-6(7H)-one (22e)

The titled compound was synthesized using the same procedure which was followed for (E)-6-Chloro-3-(3-(4-methoxyphenyl)acryloyl)-4-methylquinolin-2(1H)-one (compound-1c) using 5-acetylthieno[2,3-b]pyridin-6(7H)-one and 6-methoxybenzaldehyde as starting materials. Yield: 59%; LCMS: m/z 312.1 [M+H]⁺.

### Step-f: Synthesis of -(5-(4-methoxyphenyl)-1-propionyl-4,5-dihydro-1H-pyrazol-3-yl)thieno[2,3-b]pyridin-6(7H)-one

The titled compound was synthesized using the same procedure which was followed for 6-chloro-3-(5-(4-methoxyphenyl)-1-propionyl-4,5-dihydro-1H-pyrazol-3-yl)-4-methylquinolin-2(1H)-one (Example-7) using (E)-5-(3-(4-methoxyphenyl)acryloyl)thieno[2,3-b]pyridin-6(7H)-one as starting material. Yield: 18%; ¹H-NMR (400 MHz, DMSO): ⊐ 8.50 (s, 1H), 7.36-7.27 (m, 2H), 7.10 (d, J=8.4 Hz, 2H), 6.87 (d, J=8.4 Hz, 2H), 5.44 (dd, J=6.3 Hz & 11.6 Hz, 1H), 3.89-3.79 (m, 2H), 3.72 (s, 3H), 2.73-2.70 (m, 2H), 1.05 (t, J=7.6 Hz, 3H), One Exchangeable proton was not observed in NMR; LCMS: m/z 382.2 [M+H]⁺.

### Example-23: Synthesis of 6-chloro-4-cyclopropyl-3-(5-(4-methoxyphenyl)-1-propionyl-4,5-dihydro-1H-pyrazol-3-yl)quinolin-2(1H)-one

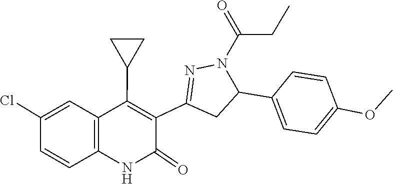 [SMILES: CCC(=O)N1N=C(c2c(C3CC3)c3cc(Cl)ccc3[nH]c2=O)CC1c1ccc(OC)cc1]

### Step-a: (2-amino-5-chlorophenyl)(cyclopropyl)methanone (23a)

The titled compound was synthesized using the same procedure which was followed for 1-(2-Amino-5-chlorophenyl)ethan-1-one (compound-1a) using 2-amino-5-chlorobenzonitrile and cyclopropylmagnesium bromide as starting materials. Yield: 14%. ¹H-NMR (300 MHz, CDCl₃): [symbol] 7.91 (d, J=2.4 Hz, 1H), 7.20 (dd, J=2.4 8.7 Hz, 1H), 6.59 (d, J=9 Hz, 1H), 615 (bs, 2H), 2.58-2.51 (m, 1H), 1.21-1.15 (m, 2H). 1.02-0.87 (m, 2H)

### Step-b: 3-acetyl-6-chloro-4-cyclopropylquinolin-2(1H)-one (23b)

The titled compound was synthesized using the same procedure which was followed for 3-Acetyl-6-chloro-4-methylquinolin-2(11H)-one (compound-1b) using (2-amino-5-chlorophenyl)(cyclopropyl)methanone as starting material. Yield: 23%; ¹H-NMR (300 MHz, CDCl₃): [symbol] 8.11 (d, J=2.1 Hz, 1H), 7.60 (dd, J=2.4, 8.7 Hz, 1H), 7.34 (d, J=8.7 Hz, 1H), 2.47 (s, 3H), 2.09-2.03 (m, 1H), 1.22 (s, 1H), 1.10-1.03 (m, 2H), 0.54-0.49 (m, 2H)

### Step-c: (E)-6-chloro-4-cyclopropyl-3-(3-(4-methoxyphenyl)acryloyl)quinolin-2(1H)-one (23c)

The titled compound was synthesized using the same procedure which was followed for (E)-6-Chloro-3-(3-(4-methoxyphenyl)acryloyl)-4-methylquinolin-2(1H)-one (compound-1c) using 3-acetyl-6-chloro-4-cyclopropylquinolin-2(1H)-one and 4-methoxybenzaldehyde as starting materials. Yield: 24%; ¹H-NMR (300 MHz, CDCl₃): [symbol] 12.42 (bs, 1H), 8.15 (d, J=5.1 Hz, 1H), 7.48 (t, J=8.7 Hz, 2H), 7.40-7.35 (m, 2H), 7.00-6.87 (m, 2H), 3.84 (s, 3H), 2.04-1.91 (m, 1H), 1.40-1.21 (m, 2H), 1.21-1.08 (m, 2H), 0.90-0.87 (m, 2H).

### Step-d: 6-chloro-4-cyclopropyl-3-(5-(4-methoxyphenyl)-1-propionyl-4,5-dihydro-1H-pyrazol-3-yl)quinolin-2(1H)-one

The titled compound was synthesized using the same procedure which was followed for 6-Chloro-3-(5-(4-methoxyphenyl)-1-propionyl-4,5-dihydro-1H-pyrazol-3-yl)-4-methylquinolin-2(1H)-one (Example-1) using (E)-6-chloro-4-cyclopropyl-3-(3-(4-methoxyphenyl)acryloyl)quinolin-2(1H)-one as starting material. Yield: 16%. ¹H-NMR (300 MHz, CDCl₃): [symbol] 13.03 (bs, 1H), 8.20 (d, J=1.5 Hz, 1H), 7.41 (d, J=8.7 Hz, 3H), 7.14 (d, J=8.7 Hz, 1H), 6.90 (d, J=5.7 Hz, 2H), 5.63 (dd, J=4.2, 11.4 Hz, 1H), 3.81 (s, 3H), 3.75-3.65 (m, 1H), 3.53 (dd, J=4.2, 18 Hz, 1H), 2.73 (q, J=7.5 Hz, 2H), 2.20-2.10 (m, 1H), 1.28-1.15 (m, 5H), 0.60-0.49 (m, 2H). LCMS (m/z): 450.2 [M+H]⁺.

### Example-24: Synthesis of 6-chloro-4-methyl-3-(5-(1-methyl-1H-indol-3-yl)-1-propionyl-4,5-dihydro-1H-pyrazol-3-yl)quinolin-2(1H)-one

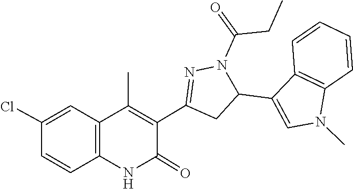 [SMILES: CCC(=O)N1N=C(c2c(C)c3cc(Cl)ccc3[nH]c2=O)CC1c1cn(C)c2ccccc12]

### Step-a: Synthesis of ((E)-6-chloro-4-methyl-3-(3-(1-methyl-1H-indol-3-yl)acryloyl)quinolin-2(1H)-one (24a)

The titled compound was synthesized using the same procedure which was followed for (E)-6-Chloro-3-(3-(4-methoxyphenyl)acryloyl)-4-methylquinolin-2(1H)-one (compound-1c) using 3-acetyl-6-chloro-4-methylquinolin-2(1H)-one and 1-methyl-1H-indole-3-carbaldehyde as starting materials. Yield: 50%; ¹H-NMR (300 MHz, DMSO-d6): ⊐ 12.34 (s, 1H), 8.08 (d, J=8.1 Hz, 2H), 7.74-7.63 (m, 4H), 7.51 (d, J=1.8 Hz, 1H), 7.37-7.32 (m, 2H), 6.97 (d, J=2.1 Hz, 1H), 6.76-6.71 (m, 1H), 4.36 (t, J=6.9 Hz, 2H), 2.42 (s, 3H).

### Step-b: Synthesis of 3-(6-chloro-4-methyl-3-(5-(1-methyl-1H-indol-3-yl)-1-propionyl-4,5-dihydro-1H-pyrazol-3-yl)-quinolin-2(1H)-one

The titled compound was synthesized using the same procedure which was followed for 6-Chloro-3-(5-(4-methoxyphenyl)-1-propionyl-4,5-dihydro-1H-pyrazol-3-yl)-4-methylquinolin-2(1H)-one (Example-1) using ((E)-6-chloro-4-methyl-3-(3-(1-methyl-1H-indol-3-yl)acryloyl)quinolin-2(1H)-one as starting material. Yield: 0.2 g. ¹H-NMR (300 MHz, DMSO-d6): [symbol] 7.83 (d, J=2.1 Hz, 1H), 7.65 (d, J=7.8 Hz, 1H), 7.57 (dd, J=2.1 Hz & 8.7 Hz, 1H), 7.42-7.33 (m, 3H), 7.15 (t, J=6.9 Hz, 1H), 7.02 (t, J=7.2 Hz, 1H), 5.05 (t, J=10.5 Hz, 1H), 3.74 (s, 3H), 3.31-3.26 (m, 1H), 3.14-3.02 (m, 1H), 2.07 (s, 3H), 1.86 (s, 1H), 1.75 (s, 1H), 1.62 (s, 1H), One exchangeable proton not observed in NMR; LCMS: m/z 447.2 [M+H]⁺

### Example-25: Synthesis of 5-(5-(5-methoxypyridin-2-yl)-1-propionyl-4,5-dihydro-1H-pyrazol-3-yl)-4-methylthieno[2,3-b]pyridin-6(7H)-one

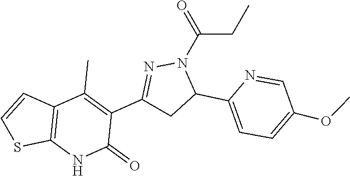 [SMILES: CCC(=O)N1N=C(c2c(C)c3ccsc3[nH]c2=O)CC1c1ccc(OC)cn1]

### Step-a: Synthesis of (E)-5-(3-(5-methoxypyridin-2-yl)acryloyl)-4-methylthieno[2,3-b]pyridin-6(7H)-one (25a)

The titled compound was synthesized using the same procedure which was followed for (E)-6-Chloro-3-(3-(4-methoxyphenyl)acryloyl)-4-methylquinolin-2(11H)-one (compound-1c) using 5-acetyl-4-methylthieno[2,3-b]pyridin-6(7H)-one and 5-methoxypicolinaldehyde as starting materials. Yield: 76%; LCMS (m/z): 327.1 [M+H]⁺.

### Step-b: Synthesis of 5-(5-(5-methoxypyridin-2-yl)-1-propionyl-4,5-dihydro-1H-pyrazol-3-yl)-4-methylthieno[2,3-b]pyridin-6(7H)-one

The titled compound was synthesized using the same procedure which was followed for 6-chloro-3-(5-(4-methoxyphenyl)-1-propionyl-4,5-dihydro-1H-pyrazol-3-yl)-4-methylquinolin-2(1H)-one (Example-7) using (E)-5-(3-(5-methoxypyridin-2-yl)acryloyl)-4-methylthieno[2,3-b]pyridin-6(7H)-one as starting material. Yield: 34%. ¹H-NMR (400 MHz, DMSO-d6): [symbol] 8.22 (d, J=2 Hz, 1H), 7.37-7.35 (m, 3H), 7.25 (d, J=8.8 Hz, 1H), 5.49 (dd, J=4.4 Hz & 7.2 Hz, 1H), 3.81 (s, 3H), 3.80-3.76 (m, 1H), 3.20 (dd, J=4.8 Hz & 13.2 Hz, 1H), 2.62 (q, J=7.6 Hz, 2H), 2.57 (s, 3H), 1.02 (t, J=7.6 Hz, 3H), One exchangeable proton not observed in NMR; LCMS (m/z): 397.2 [M+H]⁺.

### Example-26: Synthesis of 4-methyl-5-(5-(6-oxo-1,6-dihydropyridin-3-yl)-1-propionyl-4,5-dihydro-1H-pyrazol-3-yl)thieno[2,3-b]pyridin-6(7H)-one

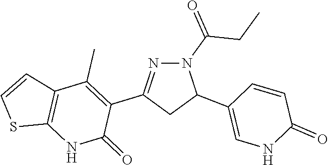 [SMILES: CCC(=O)N1N=C(c2c(C)c3ccsc3[nH]c2=O)CC1c1ccc(=O)[nH]c1]

### Step-a: Synthesis of (E)-4-methyl-5-(3-(6-oxo-1,6-dihydropyridin-3-yl)acryloyl)thieno[2,3-b]pyridin-6(7H)-one (26a)

The titled compound was synthesized using the same procedure which was followed for (E)-6-Chloro-3-(3-(4-methoxyphenyl)acryloyl)-4-methylquinolin-2(11H)-one (compound-1c) using 5-acetyl-4-methylthieno[2,3-b]pyridin-6(7H)-one and 6-oxo-1,6-dihydropyridine-3-carbaldehyde as starting materials. Yield: 80% LCMS (m/z): 313.1 [M+H]⁺.

### Step-b: Synthesis of 4-methyl-5-(5-(6-oxo-1,6-dihydropyridin-3-yl)-1-propionyl-4,5-dihydro-1H-pyrazol-3-yl)thieno[2,3-b]pyridin-6(7H)-one

The titled compound was synthesized using the same procedure which was followed for 6-chloro-3-(5-(4-methoxyphenyl)-1-propionyl-4,5-dihydro-1H-pyrazol-3-yl)-4-methylquinolin-2(1H)-one (Example-7) using (E)-4-methyl-5-(3-(6-oxo-1,6-dihydropyridin-3-yl)acryloyl)thieno[2,3-b]pyridin-6(7H)-one as starting material. Yield: 56%. ¹H-NMR (400 MHz, DMSO-d6): [symbol] 7.44 (d, J=9.2 Hz, 1H), 7.34-7.32 (m, 3H), 6.32 (d, J=9.6 Hz, 1H), 5.30 (dd, J=3.81 Hz & 7.6 Hz, 1H), 3.65-3.61 (m, 1H), 3.21 (dd, J=4.4 Hz & 14.0 Hz, 1H), 2.56 (q, J=7.6 Hz, 2H), 2.52 (s, 3H), 1.02 (t, J=7.6 Hz, 3H), (Exchangeable protons not visible in NMR); LCMS (m/z): 383.1 [M+H]⁺.

### Example-27: Synthesis of 5-(5-(4-fluorophenyl)-1-propionyl-4,5-dihydro-1H-pyrazol-3-yl)-4-methylthieno[2,3-b]pyridin-6(7H)-one

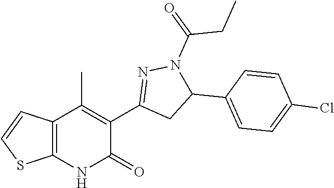 [SMILES: CCC(=O)N1N=C(c2c(C)c3ccsc3[nH]c2=O)CC1c1ccc(Cl)cc1]

### Step-a: Synthesis of (E)-5-(3-(4-fluorophenyl)acryloyl)-4-methylthieno[2,3-b]pyridin-6(7H)-one (27a)

The titled compound was synthesized using the same procedure which was followed for (E)-6-Chloro-3-(3-(4-methoxyphenyl)acryloyl)-4-methylquinolin-2(1H)-one (compound-1c) using 5-acetyl-4-methylthieno[2,3-b]pyridin-6(7H)-one and 4-fluorobenzaldehyde as starting materials. Yield: 66%, LCMS (m/z): 314.0 [M+H]⁺.

### Step-b: Synthesis of 5-(5-(4-fluorophenyl)-1-propionyl-4,5-dihydro-1H-pyrazol-3-yl)-4-methylthieno[2,3-b]pyridin-6(7H)-one

The titled compound was synthesized using the same procedure which was followed for 6-chloro-3-(5-(4-methoxyphenyl)-1-propionyl-4,5-dihydro-1H-pyrazol-3-yl)-4-methylquinolin-2(1H)-one (Example-7) using (E)-5-(3-(4-fluorophenyl)acryloyl)-4-methylthieno[2,3-b]pyridin-6(7H)-one as starting material. Yield: 12%. ¹H-NMR (400 MHz, DMSO-d6): [symbol] 7.37-7.30 (m, 4H), 7.19-7.14 (m, 2H), 5.49 (dd, J=4.4 Hz & 7.6 Hz, 1H), 3.77 (dd, J=6.4 Hz & 12 Hz, 1H), 3.14 (dd, J=4.4 Hz & 14.0 Hz, 1H), 2.66 (q, J=7.2 Hz, 2H), 2.54 (s, 3H), 1.03 (t, J=6.4 Hz, 3H), One exchangeable proton not observed in NMR; LCMS (m/z): 384.1 [M+H]⁺.

### Example-28: Synthesis of 5-(5-(4-chlorophenyl)-1-propionyl-4,5-dihydro-1H-pyrazol-3-yl)-4-methylthieno[2,3-b]pyridin-6(7H)-one

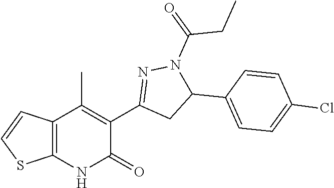 [SMILES: CCC(=O)N1N=C(c2c(C)c3ccsc3[nH]c2=O)CC1c1ccc(Cl)cc1]

### Step-a: (E)-5-(3-(4-chlorophenyl)acryloyl)-4-methylthieno[2,3-b]pyridin-6(7H)-one (28a)

The titled compound was synthesized using the same procedure which was followed for (E)-6-Chloro-3-(3-(4-methoxyphenyl)acryloyl)-4-methylquinolin-2(1H)-one (compound-1c) using 5-acetyl-4-methylthieno[2,3-b]pyridin-6(7H)-one and 4-chlorobenzaldehyde as starting materials. Yield: 94%. ¹H-NMR (400 MHz, DMSO-d6): [symbol] 7.77 (d, J=8.0 Hz, 2H), 7.47 (d, J=2.0 Hz & 8.4 Hz, 2H), 7.46-7.42 (m, 1H), 7.35-7.32 (m, 2H), 7.14 (d, J=16.0 Hz, 1H), 2.34 (s, 3H), One exchangeable proton not observed in NMR; LCMS (m/z): 330.0 [M+H]⁺.

### Step-b: Synthesis of 5-(5-(4-chlorophenyl)-1-propionyl-4,5-dihydro-1H-pyrazol-3-yl)-4-methylthieno[2,3-b]pyridin-6(7H)-one

The titled compound was synthesized using the same procedure which was followed for 6-chloro-3-(5-(4-methoxyphenyl)-1-propionyl-4,5-dihydro-1H-pyrazol-3-yl)-4-methylquinolin-2(1H)-one (Example-7) using (E)-5-(3-(4-chlorophenyl)acryloyl)-4-methylthieno[2,3-b]pyridin-6(7H)-one as starting material. Yield: 27%. ¹H-NMR (400 MHz, DMSO-d6): ⊐ 12.51 (s, 1H), 7.42 (d, J=8.4 Hz, 2H), 7.33-7.28 (m, 4H), 5.49 (dd, J=4.4 Hz & 7.6 Hz, 1H), 3.77 (dd, J=6.4 Hz & 12 Hz, 1H), 3.14 (dd, J=4.4 Hz & 14.0 Hz, 1H), 2.68 (q, J=7.2 Hz, 2H), 2.54 (s, 3H), 1.06 (t, J=6.4 Hz, 3H). LCMS (m/z): 400.0 [M+H]⁺.

### Chiral Purification of Compound 28.

Step-e: 28(A): HPLC using a chiral pack IA 5 uM, 4.6*250 mm, IPA (100) eluent at flow rate of 0.7 mL/min at 20° C. The isolated enantiomer at 19.02 min.

Step-f: 28 (B): HPLC using a chiral pack IA 5 uM, 4.6*250 mm, IPA/DCM (90/10) eluent at flow rate of 0.7 mL/min at 20° C. The isolated enantiomer at 26.79 min.

### Example-29: Synthesis of 5-(5-(5-methoxythiophen-2-yl)-1-propionyl-4,5-dihydro-1H-pyrazol-3-yl)-4-methylthieno[2,3-b]pyridin-6(7H)-one

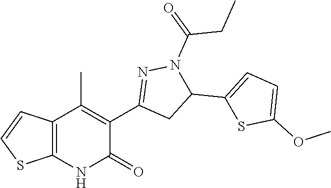 [SMILES: CCC(=O)N1N=C(c2c(C)c3ccsc3[nH]c2=O)CC1c1ccc(OC)s1]

### Step-a: Synthesis of (E)-5-(3-(5-methoxythiophen-2-yl)acryloyl)-4-methylthieno[2,3-b]pyridin-6(7H)-one (29a)

The titled compound was synthesized using the same procedure which was followed for (E)-6-Chloro-3-(3-(4-methoxyphenyl)acryloyl)-4-methylquinolin-2(11H)-one (compound-1c) using 5-acetyl-4-methylthieno[2,3-b]pyridin-6(7H)-one and 5-methoxythiophene-2-carbaldehyde as starting materials. Yield: 63%; LCMS (m/z): 332.0 [M+H]⁺.

### Step-b: Synthesis of 5-(5-(5-methoxythiophen-2-yl)-1-propionyl-4,5-dihydro-1H-pyrazol-3-yl)-4-methylthieno[2,3-b]pyridin-6(7H)-one

The titled compound was synthesized using the same procedure which was followed for 6-chloro-3-(5-(4-methoxyphenyl)-1-propionyl-4,5-dihydro-1H-pyrazol-3-yl)-4-methylquinolin-2(1H)-one (Example-7) using (E)-5-(3-(5-methoxythiophen-2-yl)acryloyl)-4-methylthieno[2,3-b]pyridin-6(7H)-one as starting material. Yield: 6%; ¹H-NMR (400 MHz, DMSO-d6): ⊐ 7.33-7.31 (m, 2H), 6.74 (d, J=4.0 Hz, 1H), 6.11 (d, J=3.6 Hz, 1H), 5.62 (dd, J₁=3.2 Hz & 10.8 Hz, 1H), 3.80 (s, 3H), 3.72 (dd, J=11.6 Hz & 18.4 Hz, 1H), 3.34 (dd, J=3.6 Hz & 17.6 Hz, 1H), 2.56 (q, J=6.8 Hz, 2H), 2.51 (s, 3H), 1.04 (t, J=7.6 Hz, 3H), One exchangeable proton not observed in NMR; LCMS (m/z): 402.2 [M+H]⁺.

### Example-30: Synthesis of 5-(5-(2-methoxythiazol-5-yl)-1-propionyl-4,5-dihydro-1H-pyrazol-3-yl)-4-methylthieno[2,3-b]pyridin-6(7H)-one

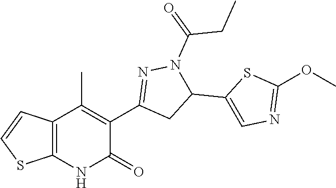 [SMILES: CCC(=O)N1N=C(c2c(C)c3ccsc3[nH]c2=O)CC1c1cnc(OC)s1]

### Step-a: Synthesis of (E)-5-(3-(2-methoxythiazol-5-yl)acryloyl)-4-methylthieno[2,3-b]pyridin-6(7H)-one (30a)

The titled compound was synthesized using the same procedure which was followed for (E)-6-Chloro-3-(3-(4-methoxyphenyl)acryloyl)-4-methylquinolin-2(1H)-one (compound-1c) using 5-acetyl-4-methylthieno[2,3-b]pyridin-6(7H)-one and 2-methoxythiazole-5-carbaldehyde as starting materials. Yield: 48%; LCMS (m/z): 332.90 [M+H]⁺.

### Step-b: Synthesis of 5-(5-(2-methoxythiazol-5-yl)-1-propionyl-4,5-dihydro-1H-pyrazol-3-yl)-4-methylthieno[2,3-b]pyridin-6(7H)-one

The titled compound was synthesized using the same procedure which was followed for 6-chloro-3-(5-(4-methoxyphenyl)-1-propionyl-4,5-dihydro-1H-pyrazol-3-yl)-4-methylquinolin-2(1H)-one (Example-7) using (E)-5-(3-(2-methoxythiazol-5-yl)acryloyl)-4-methylthieno[2,3-b]pyridin-6(7H)-one as starting material. Yield: 5%; ¹H-NMR (400 MHz, DMSO-d6): ⊐ 7.33-7.31 (m, 2H), 7.23 (s, 1H), 5.68 (dd, J=12.0 Hz & 18.0 Hz, 1H), 3.97 (s, 3H), 3.72 (dd, J=11.6 Hz & 18.4 Hz, 1H), 3.34 (dd, J=3.2 Hz & 3.6 Hz, 1H), 2.56 (q, J=6.8 Hz, 2H), 2.51 (s, 3H), 1.04 (t, J=7.6 Hz, 3H), One exchangeable proton not observed in NMR; LCMS (m/z): 402.90 [M+H]⁺.

### Example-31: Synthesis of 5-(5-(5-bromothiophen-3-yl)-1-propionyl-4,5-dihydro-1H-pyrazol-3-yl)-4-methylthieno[2,3-b]pyridin-6(7H)-one

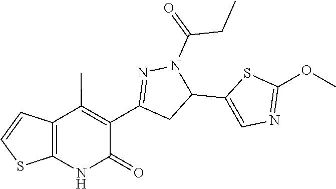 [SMILES: CCC(=O)N1N=C(c2c(C)c3ccsc3[nH]c2=O)CC1c1cnc(OC)s1]

### Step-a: Synthesis of (E)-5-(3-(5-bromothiophen-3-yl)acryloyl)-4-methylthieno[2,3-b]pyridin-6(7H)-one (31a)

The titled compound was synthesized using the same procedure which was followed for (E)-6-chloro-3-(3-(4-methoxyphenyl)acryloyl)-4-methylquinolin-2(11H)-one (compound-1c) using 5-acetyl-4-methylthieno[2,3-b]pyridin-6(7H)-one and 5-bromothiophene-3-carbaldehyde as starting materials. Yield: 36%; LCMS (m/z): 382.0 [M+2H]⁺.

### Step-b: Synthesis of 5-(5-(5-bromothiophen-3-yl)-1-propionyl-4,5-dihydro-1H-pyrazol-3-yl)-4-methylthieno[2,3-b]pyridin-6(7H)-one

The titled compound was synthesized using the same procedure which was followed for 6-chloro-3-(5-(4-methoxyphenyl)-1-propionyl-4,5-dihydro-1H-pyrazol-3-yl)-4-methylquinolin-2(1H)-one (Example-7) using (E)-5-(3-(5-bromothiophen-3-yl)acryloyl)-4-methylthieno[2,3-b]pyridin-6(7H)-one as starting material. Yield: 5%; ¹H-NMR (400 MHz, DMSO-d6): [symbol] 7.42 (s, 1H), 7.35-7.33 (m, 2H), 7.21 (d, J=0.8 Hz, 1H), 5.53 (dd, J=3.2 Hz & 11.2 Hz, 1H), 3.62 (dd, J=11.6 Hz & 17.6 Hz, 1H), 3.25 (dd, J=3.6 Hz & 18.0 Hz, 1H), 2.61 (q, J=7.6 Hz, 2H), 2.50 (s, 3H), 1.03 (t, J=8.0 Hz, 3H), One exchangeable proton not observed in NMR; LCMS (m/z): 452.1 [M+2H]⁺.

### Example-32: Synthesis of 4-methyl-5-(5-phenyl-1-propionyl-4,5-dihydro-1H-pyrazol-3-yl)thieno[2,3-b]pyridin-6(7H)-one

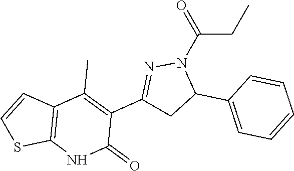 [SMILES: CCC(=O)N1N=C(c2c(C)c3ccsc3[nH]c2=O)CC1c1ccccc1]

### Step-a: Synthesis of 5-cinnamoyl-4-methylthieno[2,3-b]pyridin-6(7H)-one (32a)

The titled compound was synthesized using the same procedure which was followed for (E)-6-Chloro-3-(3-(4-methoxyphenyl)acryloyl)-4-methylquinolin-2(1H)-one (compound-1c) using 5-acetyl-4-methylthieno[2,3-b]pyridin-6(7H)-one and benzaldehyde as starting materials. Yield: 98%, ¹H-NMR (400 MHz, DMSO-d6): [symbol] 7.74-7.71 (m, 2H), 7.40-7.45 (m, 4H), 7.39-7.35 (m, 2H), 7.12 (d, J=16.4 Hz, 1H), 2.35 (s, 3H), One exchangeable proton not observed in NMR; LCMS (m/z): 296.1 [M+H]⁺.

### Step-b: Synthesis of 4-methyl-5-(5-phenyl-1-propionyl-4,5-dihydro-1H-pyrazol-3-yl)thieno[2,3-b]pyridin-6(7H)-one

The titled compound was synthesized using the same procedure which was followed for 6-chloro-3-(5-(4-methoxyphenyl)-1-propionyl-4,5-dihydro-1H-pyrazol-3-yl)-4-methylquinolin-2(1H)-one (Example-7) using 5-cinnamoyl-4-methylthieno[2,3-b]pyridin-6(7H)-one as starting material. Yield: Yield: 37%; ¹H-NMR (400 MHz, DMSO-d6): [symbol] 7.36-7.32 (m, 4H), 7.27-7.25 (m, 3H), 5.48 (dd, J=4.0 Hz & 11.6 Hz, 1H), 3.80 (dd, J=12.0 Hz &18.4 Hz, 1H), 3.14 (dd, J=4.4 Hz & 18.4 Hz, 1H), 2.66 (q, J=7.2 Hz, 2H), 2.55 (s, 3H), 1.04 (t, J=7.2 Hz, 3H), [NH proton is not visible in NMR]; LCMS (m/z): 366.2 [M+H]⁺.

### Example-32: Synthesis of 4-methyl-5-(1-propionyl-5-(thiophen-3-yl)-4,5-dihydro-1H-pyrazol-3-yl)thieno[2,3-b]pyridin-6(7H)-one

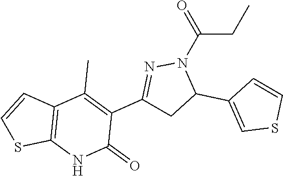 [SMILES: CCC(=O)N1N=C(c2c(C)c3ccsc3[nH]c2=O)CC1c1ccsc1]

### Step-a: Synthesis of (E)-4-methyl-5-(3-(thiophen-3-yl)acryloyl)thieno[2,3-b]pyridin-6(7H)-one (33a)

The titled compound was synthesized using the same procedure which was followed for (E)-6-Chloro-3-(3-(4-methoxyphenyl)acryloyl)-4-methylquinolin-2(1H)-one (compound-1c) using 5-acetyl-4-methylthieno[2,3-b]pyridin-6(7H)-one and thiophene-3-carbaldehyde as starting materials. Yield: 96%. ¹H-NMR (400 MHz, DMSO-d6): ⊐ 12.24 (bs, 1H) 8.17 (d, J=2.0 Hz, 1H), 7.64-7.57 (m, 2H), 7.42-7.38 (m, 1H), 7.34-7.31 (m, 2H), 6.91 (d, J=16.4 Hz, 1H), 2.32 (s, 3H); LCMS (m/z): 302.0 [M+H]⁺.

### Step-b: Synthesis of 4-methyl-5-(1-propionyl-5-(thiophen-3-yl)-4,5-dihydro-1H-pyrazol-3-yl)thieno[2,3-b]pyridin-6(7H)-one

The titled compound was synthesized using the same procedure which was followed for 6-chloro-3-(5-(4-methoxyphenyl)-1-propionyl-4,5-dihydro-1H-pyrazol-3-yl)-4-methylquinolin-2(1H)-one (Example-7) using (E)-4-methyl-5-(3-(thiophen-3-yl)acryloyl)thieno[2,3-b]pyridin-6(7H)-one as starting material. Yield: 35%. ¹H-NMR (400 MHz, DMSO-d6): [symbol] 7.50-7.48 (m, 1H), 7.41-7.40 (m, 1H), 7.34-7.33 (m, 2H), 7.10 (d, J=4.0 Hz, 1H), 5.60 (dd, J=5.0 Hz & 15.2 Hz, 1H), 3.69-3.68 (m, 1H), 3.25 (dd, J=3.2 Hz & 15.2 Hz, 1H), 2.61 (q, J=7.2 Hz, 2H), 2.50 (s, 3H), 1.03 (t, J=8.0 Hz, 3H), [NH proton is not visible in NMR]; LCMS (m/z): 372.0 [M+H]⁺.

### Chiral Purification of Compound 33.

Step-e: 33 (A): HPLC using a chiral pack IA 5 uM, 4.6*250 mm, IPA (100) eluent at flow rate of 0.7 mL/min at 20° C. The isolated enantiomer at 16.33 min.

Step-f: 33 (B): HPLC using a chiral pack IA 5 uM, 4.6*250 mm, IPA/DCM (90/10) eluent at flow rate of 0.7 mL/min at 20° C. The isolated enantiomer at 21.93 min.

### Example-34: Synthesis of 5-(5-(4-bromothiophen-2-yl)-1-propionyl-4,5-dihydro-1H-pyrazol-3-yl)-4-methylthieno[2,3-b]pyridin-6(7H)-one

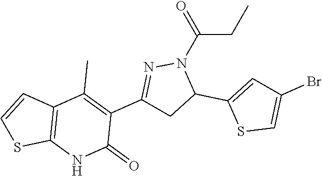 [SMILES: CCC(=O)N1N=C(c2c(C)c3ccsc3[nH]c2=O)CC1c1cc(Br)cs1]

### Step-a: Synthesis of ((E)-5-(3-(4-bromothiophen-2-yl)acryloyl)-4-methylthieno[2,3-b]pyridin-6(7H)-one (34a)

The titled compound was synthesized using the same procedure which was followed for (E)-6-Chloro-3-(3-(4-methoxyphenyl)acryloyl)-4-methylquinolin-2(11H)-one (compound-1c) using 5-acetyl-4-methylthieno[2,3-b]pyridin-6(7H)-one and 4-bromothiophene-2-carbaldehyde as starting materials. Yield: 95%. LCMS (m/z): 481.9 [M+2H]⁺

### Step-b: Synthesis of 5-(5-(4-bromothiophen-2-yl)-1-propionyl-4,5-dihydro-1H-pyrazol-3-yl)-4-methylthieno[2,3-b]pyridin-6(7H)-one

The titled compound was synthesized using the same procedure which was followed for 6-chloro-3-(5-(4-methoxyphenyl)-1-propionyl-4,5-dihydro-1H-pyrazol-3-yl)-4-methylquinolin-2(1H)-one (Example-7) using ((E)-5-(3-(4-bromothiophen-2-yl)acryloyl)-4-methylthieno[2,3-b]pyridin-6(7H)-one as starting material. Yield: 35%. 1H-NMR (400 MHz, DMSO-d6): ⊐ 7.56 (s, 1H), 7.33 (bs, 2H), 7.14 (s, 1H), 5.77 (d, J=8.0 Hz, 1H), 3.76 (dd, J=12 Hz & 18.4 Hz, 1H), 3.37 (bs, 1H), 2.63-2.58 (m, 2H), 2.53 s, 3H), 1.05 (t, J=7.2 Hz, 3H), one Exchangeable proton not observed in NMR; LCMS (m/z): 452.0 [M+2H]⁺.

### Example-35: Synthesis of 4-methyl-5-(1-propionyl-5-(pyridin-4-yl)-4,5-dihydro-1H-pyrazol-3-yl)thieno[2,3-b]pyridin-6(7H)-one

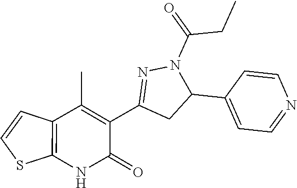 [SMILES: CCC(=O)N1N=C(c2c(C)c3ccsc3[nH]c2=O)CC1c1ccncc1]

### Step-a: Synthesis of (E)-4-methyl-5-(3-(pyridin-4-yl)acryloyl)thieno[2,3-b]pyridin-6(7H)-one (35a)

The titled compound was synthesized using the same procedure which was followed for (E)-6-Chloro-3-(3-(4-methoxyphenyl)acryloyl)-4-methylquinolin-2(1H)-one (compound-1c) using 5-acetyl-4-methylthieno[2,3-b]pyridin-6(7H)-one and isonicotinaldehyde as starting materials. Yield: 60%; LCMS (m/z): 297.10 [M+H]⁺.

### Step-b: Synthesis of 4-methyl-5-(1-propionyl-5-(pyridin-4-yl)-4,5-dihydro-1H-pyrazol-3-yl)thieno[2,3-b]pyridin-6(7H)-one (35b)

The titled compound was synthesized using the same procedure which was followed for 6-chloro-3-(5-(4-methoxyphenyl)-1-propionyl-4,5-dihydro-1H-pyrazol-3-yl)-4-methylquinolin-2(1H)-one (Example-7) using (E)-4-methyl-5-(3-(pyridin-4-yl)acryloyl)thieno[2,3-b]pyridin-6(7H)-one as starting material. Yield: 5%; ¹H-NMR (400 MHz, DMSO-d6): ⊐ 8.55 (d, J=5.6 Hz, 2H), 7.35-732 (m, 2H), 7.26 (d, J=6.0 Hz, 2H), 5.52 (dd, J₁=4.4 Hz & 12.0 Hz, 1H), 3.85 (dd, J₁=12.0 Hz & 18.0 Hz, 1H), 3.17 (dd, J=4.4 Hz & 18.0 Hz, 1H), 2.62 (q, J=6.8 Hz, 2H), 2.55 (s, 3H), 1.04 (t, J=7.6 Hz, 3H), One exchangeable proton not observed in NMR; LCMS (m/z): 367.2 M+H]⁺.

### Example-36: Synthesis of 4-methyl-5-(1-propionyl-5-(pyridin-3-yl)-4,5-dihydro-1H-pyrazol-3-yl)thieno[2,3-b]pyridin-6(7H)-one

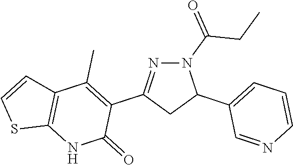 [SMILES: CCC(=O)N1N=C(c2c(C)c3ccsc3[nH]c2=O)CC1c1cccnc1]

### Step-a: Synthesis of (E)-4-methyl-5-(3-(pyridin-3-yl)acryloyl)thieno[2,3-b]pyridin-6(7H)-one (36a)

The titled compound was synthesized using the same procedure which was followed for (E)-6-Chloro-3-(3-(4-methoxyphenyl)acryloyl)-4-methylquinolin-2(1H)-one (compound-1c) using 5-acetyl-4-methylthieno[2,3-b]pyridin-6(7H)-one and nicotinaldehyde as starting materials. Yield: 98%. LCMS (m/z): 297.1 [M+H]⁺.

### Step-b: Synthesis of 4-methyl-5-(1-propionyl-5-(pyridin-3-yl)-4,5-dihydro-1H-pyrazol-3-yl)thieno[2,3-b]pyridin-6(7H)-one

The titled compound was synthesized using the same procedure which was followed for 6-chloro-3-(5-(4-methoxyphenyl)-1-propionyl-4,5-dihydro-1H-pyrazol-3-yl)-4-methylquinolin-2(1H)-one (Example-7) using (E)-4-methyl-5-(3-(pyridin-3-yl)acryloyl)thieno[2,3-b]pyridin-6(7H)-one as starting material. Yield: 23%. ¹H-NMR (400 MHz, DMSO-d6): [symbol] 12.51 (bs, 1H), 8.53 (d, J=2.0 Hz, 1H), 8.48 (dd, J=1.6 Hz & 4.8 Hz, 1H), 7.68 (d, J=7.6 Hz, 1H), 7.40-7.32 (m, 3H), 5.55 (dd, J=4.4 Hz & 11.6 Hz, 1H), 3.83 (dd, J=11.6 Hz &18.4 Hz, 1H), 3.24 (dd, J=4.8 Hz & 19.2 Hz, 1H), 2.64 (q, J=7.6 Hz, 2H), 2.50 (s, 3H), 1.03 (t, J=7.6 Hz, 3H). LCMS (m/z): 367.1 [M+H]⁺.

### Example-37: Synthesis of 4-methyl-5-(1-propionyl-5-(p-tolyl)-4,5-dihydro-1H-pyrazol-3-yl)thieno[2,3-b]pyridin-6(7H)-one

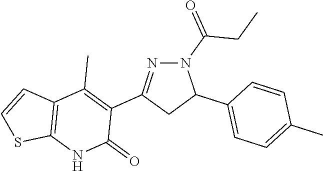 [SMILES: CCC(=O)N1N=C(c2c(C)c3ccsc3[nH]c2=O)CC1c1ccc(C)cc1]

### Step-a: Synthesis of (E)-4-methyl-5-(3-(pyridin-3-yl)acryloyl)thieno[2,3-b]pyridin-6(7H)-one (37a)

The titled compound was synthesized using the same procedure which was followed for (E)-6-Chloro-3-(3-(4-methoxyphenyl)acryloyl)-4-methylquinolin-2(1H)-one (compound-1c) using 5-acetyl-4-methylthieno[2,3-b]pyridin-6(7H)-one and 4-methylbenzaldehyde as starting materials. Yield: 96%, ¹H-NMR (400 MHz, DMSO-d6): ⊐ 12.24 (bs, 1H), 7.60 (d, J=8.0 Hz, 2H), 7.38 (d, J=16.4 Hz, 1H), 7.33-7.30 (m, 2H), 7.22 (d, J=8.0 Hz, 2H), 7.06 (d, J=16.4 Hz, 1H), 2.33 (s, 6H); LCMS (m/z): 310.2 [M+H]⁺

### Step-b: Synthesis of 4-methyl-5-(1-propionyl-5-(p-tolyl)-4,5-dihydro-1H-pyrazol-3-yl)thieno[2,3-b]pyridin-6(7H)-one

The titled compound was synthesized using the same procedure which was followed for 6-chloro-3-(5-(4-methoxyphenyl)-1-propionyl-4,5-dihydro-1H-pyrazol-3-yl)-4-methylquinolin-2(1H)-one (Example-7) using (E)-4-methyl-5-(3-(pyridin-3-yl)acryloyl)thieno[2,3-b]pyridin-6(7H)-one as starting material. Yield: 37%. ¹H-NMR (400 MHz, DMSO-d6): ⊐ 7.35-7.34 (m, 2H), 7.19-7.14 (m, 4H), 5.44 (dd, J=4.0 Hz & 11.6 Hz, 1H), 3.76 (dd, J=11.6 Hz &18.0 Hz, 1H), 3.10 (dd, J=4.4 Hz & 18.4 Hz, 1H), 2.66 (q, J=7.2 Hz, 2H), 2.54 (s, 3H), 2.27 (s, 3H), 1.03 (t, J=7.6 Hz, 3H), One exchangeable proton not observed in NMR; LCMS (m/z): 380.1 [M+H]⁺.

### Chiral Purification of Compound 37.

Step-e: 37 (A): HPLC using a chiral pack IA 5 uM, 4.6*250 mm, IPA (100) eluent at flow rate of 0.7 mL/min at 20° C. The isolated enantiomer at 13.13 min, (the levo (−) isomer).

Step-f: 37 (B): HPLC using a chiral pack IA 5 uM, 4.6*250 mm, IPA/DCM (90/10) eluent at flow rate of 0.7 mL/min at 20° C. The isolated enantiomer at 18.87 min, (the dextro (+) isomer).

### Example-38: Synthesis of 4-methyl-5-(5-(4-(4-methylpiperazin-1-yl)phenyl)-1-propionyl-4,5-dihydro-1H-pyrazol-3-yl)thieno[2,3-b]pyridin-6(7H)-one

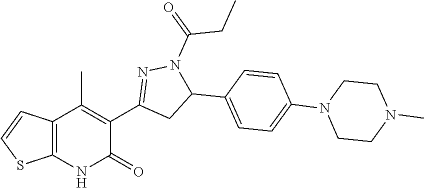 [SMILES: CCC(=O)N1N=C(c2c(C)c3ccsc3[nH]c2=O)CC1c1ccc(N2CCN(C)CC2)cc1]

### Step-a: Synthesis of (E)-4-methyl-5-(3-(4-(4-methylpiperazin-1-yl)phenyl)acryloyl)thieno[2,3-b]pyridin-6(7H)-one

The titled compound was synthesized using the same procedure which was followed for (E)-6-Chloro-3-(3-(4-methoxyphenyl)acryloyl)-4-methylquinolin-2(1H)-one (compound-1c) using 5-acetyl-4-methylthieno[2,3-b]pyridin-6(7H)-one and 4-(4-methyl piperazin-1-yl)benzaldehyde as starting materials. Yield: 79%; LCMS: m/z 393.51 [M+H]⁺.

### Step-b: Synthesis of 4-methyl-5-(5-(4-(4-methylpiperazin-1-yl)phenyl)-1-propionyl-4,5-dihydro-1H-pyrazol-3-yl)thieno[2,3-b]pyridin-6(7H)-one

The titled compound was synthesized using the same procedure which was followed for 6-chloro-3-(5-(4-methoxyphenyl)-1-propionyl-4,5-dihydro-1H-pyrazol-3-yl)-4-methylquinolin-2(1H)-one (Example-7) using (E)-4-methyl-5-(3-(4-(4-methylpiperazin-1-yl)phenyl)acryloyl)thieno[2,3-b]pyridin-6(7H)-one as starting material. Yield: 11%; ¹H-NMR (400 MHz, DMSO-d6): [symbol] 12.45 (bs, H), 7.32-7.31 (m, 2H), 7.12 (d, J=8.8 Hz, 2H), 6.883 (d, J=8.4 Hz, 2H), 5.381 (dd, J=4.4 Hz & J=11.6 Hz, 1H), 3.72 (m, 1H), 3.28 (m, 1H), 3.115 (m, 5H), 2.58 (s, 3H), 2.60 (m, 2H), 2.537 (s, 3H), 2.43 (m, 4H), 2.208 (s, 3H), 1.33 (s, 1H), 1.027 (t, J=7.6 Hz, 3H); LCMS: m/z 464.20 [M+H]⁺.

### Example-39: Synthesis of 5-(5-(4-(dimethylamino)phenyl)-1-propionyl-4,5-dihydro-1H-pyrazol-3-yl)-4-methylthieno[2,3-b]pyridin-6(7H)-one

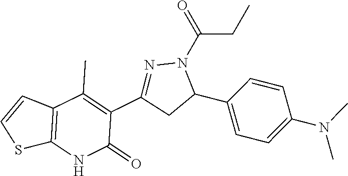 [SMILES: CCC(=O)N1N=C(c2c(C)c3ccsc3[nH]c2=O)CC1c1ccc(N(C)C)cc1]

### Step-a: Synthesis of (E)-5-(3-(4-(dimethylamino)phenyl)acryloyl)-4-methylthieno[2,3-b]pyridin-6(7H)-one

The titled compound was synthesized using the same procedure which was followed for (E)-6-Chloro-3-(3-(4-methoxyphenyl)acryloyl)-4-methylquinolin-2(1H)-one (compound-1c) using 5-acetyl-4-methylthieno[2,3-b]pyridin-6(7H)-one and 4-(dimethylamino)benzaldehyde as starting materials. Yield: 53%; LCMS: m/z 339.48[M+H]⁺.

### Step-b: Synthesis of 5-(5-(4-(dimethylamino)phenyl)-1-propionyl-4,5-dihydro-1H-pyrazol-3-yl)-4-methylthieno[2,3-b]pyridin-6(7H)-on

The titled compound was synthesized using the same procedure which was followed for 6-chloro-3-(5-(4-methoxyphenyl)-1-propionyl-4,5-dihydro-1H-pyrazol-3-yl)-4-methylquinolin-2(1H)-one (Example-7) using (E)-5-(3-(4-(dimethylamino)phenyl)acryloyl)-4-methylthieno[2,3-b]pyridin-6(7H)-one as starting material. Yield: 11%; ¹H-NMR (400 MHz, DMSO-d6): □ 7.33 (s, 2H), 7.10 (d, J=8.4 Hz, 2H), 6.67 (d, J=8.8 Hz, 2H), 5.37 (dd, J=4 Hz & J=11.2 Hz, 1H), 3.71 (m, 1H), 3.11 (dd, J=4.4 Hz & J=18.4 Hz, 1H), 2.86 (s, 6H), 2.59 (m, 2H), 2.54 (s, 3H), 1.02 (t, J=7.6 Hz, 3H), [NH proton is not visible in NMR]; LCMS: m/z 409.30[M+H]⁺.

### Example-40: Synthesis of 3-chloro-5-(5-(4-methoxyphenyl)-1-propionyl-4,5-dihydro-1H-pyrazol-3-yl)-4-methylthieno[2,3-b]pyridin-6(7H)-one

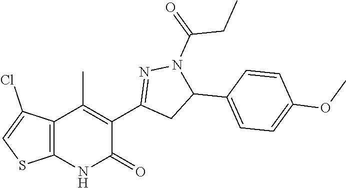 [SMILES: CCC(=O)N1N=C(c2c(C)c3c(Cl)csc3[nH]c2=O)CC1c1ccc(OC)cc1]

### Step-a: synthesis of 5-acetyl-3-chloro-4-methylthieno[2,3-b]pyridin-6(7H)-one

To a stirred solution of 5-acetyl-4-methylthieno[2,3-b]pyridin-6(7H)-one (0.12 g, 0.58 mmol) in DMF (5 mL) at 0° C. was added N-chlorosuccinimide (0.85 g 0.638 mmol). The reaction mixture was stirred at room temperature for 16 h, and progress of the reaction was monitored by TLC. Reaction mixture was diluted with water (10 mL), extracted with ethyl acetate (2×20 mL). The combined organic layer was washed with brine (15 mL), dried over Na₂SO₄ and evaporated under reduced pressure to get the crude compound which was purified by flash column chromatography with 10-20% ethyl acetate in hexane as eluent to afford the titled compound. Yield: 0.1 g; LCMS: m/z 242.10[M+H]⁺.

### Step-b: Synthesis of (E)-3-chloro-5-(3-(4-methoxyphenyl)acryloyl)-4-methylthieno[2,3-b]pyridin-6(7H)-one

The titled compound was synthesized using the same procedure which was followed for (E)-6-Chloro-3-(3-(4-methoxyphenyl)acryloyl)-4-methylquinolin-2(1H)-one (compound-1c) using 5-acetyl-3-chloro-4-methylthieno[2,3-b]pyridin-6(7H)-one and 4-methoxy benzaldehyde as starting materials. LCMS: m/z 360.10 [M+H]⁺.

### Step-c: Synthesis of 3-chloro-5-(5-(4-methoxyphenyl)-1-propionyl-4,5-dihydro-1H-pyrazol-3-yl)-4-methylthieno[2,3-b]pyridin-6(7H)-one

The titled compound was synthesized using the same procedure which was followed for 6-chloro-3-(5-(4-methoxyphenyl)-1-propionyl-4,5-dihydro-1H-pyrazol-3-yl)-4-methylquinolin-2(1H)-one (Example-7) using (E)-3-chloro-5-(3-(4-methoxyphenyl)acryloyl)-4-methylthieno[2,3-b]pyridin-6(7H)-one as starting material. Yield: 4%; ¹H-NMR (400 MHz, DMSO-d6): □ 7.45 (s, 1H), 7.20 (d, J=8.8 Hz, 2H), 6.88 (d, J=8.8 Hz, 2H), 5.42 (dd, J=4 Hz & J=11.6 Hz, 1H), 3.732 (m, 4H), 3.10 (dd, J=4.4 Hz & J=18.4 Hz, 1H), 2.67-2.55 (q, 2H), 2.52-2.5 (s, 3H), 1.025 (t, =7.6 Hz, 3H), [NH proton is not visible in NMR]; LCMS: m/z 430.30 [M+H]⁺.

### Example-41: Synthesis of 5-(5-(5-methylthiophen-2-yl)-1-propionyl-4,5-dihydro-1H-pyrazol-3-yl)-4-methylthieno[2,3-b]pyridin-6(7H)-one

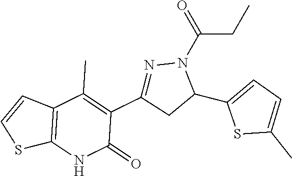 [SMILES: CCC(=O)N1N=C(c2c(C)c3ccsc3[nH]c2=O)CC1c1ccc(C)s1]

### Step-a: Synthesis of (E)-4-methyl-5-(3-(5-methylthiophen-2-yl)acryloyl)thieno[2,3-b]pyridin-6(7H)-one

The titled compound was synthesized using the same procedure which was followed for (E)-6-Chloro-3-(3-(4-methoxyphenyl)acryloyl)-4-methylquinolin-2(1H)-one (compound-1c) using 5-acetyl-4-methylthieno[2,3-b]pyridin-6(7H)-one and 5-methylthiophene-2-carbaldehyde as starting materials. Yield: 52%; LCMS: m/z 316.00 [M+H]⁺.

### Step-b: Synthesis of 5-(5-(5-methylthiophen-2-yl)-1-propionyl-4,5-dihydro-1H-pyrazol-3-yl)-4-methylthieno[2,3-b]pyridin-6(7H)-one

The titled compound was synthesized using the same procedure which was followed for 6-chloro-3-(5-(4-methoxyphenyl)-1-propionyl-4,5-dihydro-1H-pyrazol-3-yl)-4-methylquinolin-2(1H)-one (Example-7) using (E)-4-methyl-5-(3-(5-methylthiophen-2-yl)acryloyl)thieno[2,3-b]pyridin-6(7H)-one as starting material. Yield: 10%; ¹H-NMR (400 MHz, DMSO-d6): □ 7.33 (s, 2H), 6.86 (d, J=3.2 Hz, 1H), 6.62 (d, J=2 Hz, 1H), 5.69 (dd, J=3.2 Hz & J=11.2 Hz, 1H), 3.761 (m, 1H), 3.29 (dd, J=4.8 Hz & 13.6 Hz, 1H), 2.58-2.50 (m, 5H), 2.38 (s, 3H), 1.043 (t, J=7.6 Hz, 3H), [NH proton is not visible in NMR]; LCMS: m/z 386.0[M+H]⁺.

### Example-42: Synthesis of 4-methyl-5-(1-propionyl-5-(4-(trifluoromethyl)phenyl)-4,5-dihydro-1H-pyrazol-3-yl)thieno[2,3-b]pyridin-6(7H)-one

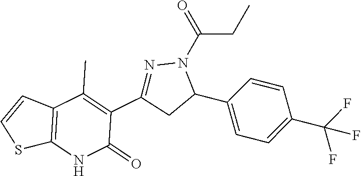 [SMILES: CCC(=O)N1N=C(c2c(C)c3ccsc3[nH]c2=O)CC1c1ccc(C(F)(F)F)cc1]

### Step-a: Synthesis of ((E)-4-methyl-5-(3-(4-(trifluoromethyl)phenyl)acryloyl)thieno[2,3-b]pyridin-6(7H)-one (42a)

The titled compound was synthesized using the same procedure which was followed for (E)-6-Chloro-3-(3-(4-methoxyphenyl)acryloyl)-4-methylquinolin-2(1H)-one (compound-1c) using 5-acetyl-4-methylthieno[2,3-b]pyridin-6(7H)-one and 4-(trifluoromethyl)benzaldehyde as starting materials. Yield: 82%. LCMS (m/z): 364.1 [M+H]⁺.

### Step-b: Synthesis of 4-methyl-5-(1-propionyl-5-(4-(trifluoromethyl)phenyl)-4,5-dihydro-1H-pyrazol-3-yl)thieno[2,3-b]pyridin-6(7H)-one

The titled compound was synthesized using the same procedure which was followed for 6-chloro-3-(5-(4-methoxyphenyl)-1-propionyl-4,5-dihydro-1H-pyrazol-3-yl)-4-methylquinolin-2(1H)-one (Example-7) using ((E)-4-methyl-5-(3-(4-(trifluoromethyl)phenyl)acryloyl)thieno[2,3-b]pyridin-6(7H)-one as starting material. Yield: 16%, ¹H-NMR (400 MHz, DMSO-d6): [symbol] 7.72 (d, J=8.4 Hz, 2H), 7.48 (d, J=8.0 Hz, 2H), 7.35-7.32 (m, 2H), 5.55 (dd, J=4.4 Hz & 7.6 Hz, 1H), 3.86 (dd, J=12.0 Hz & 18.4 Hz, 1H), 3.16 (dd, J=4.8 Hz & 13.6 Hz, 1H), 2.69-2.64 (m, 2H), 2.56 (s, 3H), 1.03 (t, J=7.6 Hz, 3H), One exchangeable proton not observed in NMR; LCMS (m/z): 434.1 [M+H]⁺.

### Example-43: Synthesis of 5-(5-(4-(difluoromethyl)phenyl)-1-propionyl-4,5-dihydro-1H-pyrazol-3-yl)-4-methylthieno[2,3-b]pyridin-6(7H)-one

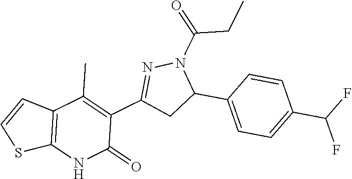 [SMILES: CCC(=O)N1N=C(c2c(C)c3ccsc3[nH]c2=O)CC1c1ccc(C(F)F)cc1]

### Step-a: Synthesis of (E)-5-(3-(4-(difluoromethyl)phenyl)acryloyl)-4-methylthieno[2,3-b]pyridin-6(7H)-one

The titled compound was synthesized using the same procedure which was followed for (E)-6-Chloro-3-(3-(4-methoxyphenyl)acryloyl)-4-methylquinolin-2(1H)-one (compound-1c) using 5-acetyl-4-methylthieno[2,3-b]pyridin-6(7H)-one and 4-(difluoromethyl) benzaldehyde as starting materials. Yield: 79%; LCMS: m/z 346.10 [M+H]⁺.

### Step-b: Synthesis of 5-(5-(4-(difluoromethyl)phenyl)-1-propionyl-4,5-dihydro-1H-pyrazol-3-yl)-4-methylthieno[2,3-b]pyridin-6(7H)-one

The titled compound was synthesized using the same procedure which was followed for 6-chloro-3-(5-(4-methoxyphenyl)-1-propionyl-4,5-dihydro-1H-pyrazol-3-yl)-4-methylquinolin-2(1H)-one (Example-7) using Synthesis of (E)-5-(3-(4-(difluoromethyl)phenyl)acryloyl)-4-methylthieno[2,3-b]pyridin-6(7H)-one as starting material. Yield: 19%; ¹H-NMR (400 MHz, DMSO-d6): □ 7.35-7.32 (m, 2H), 7.40 (d, J=3.2 Hz 1H), 6.26 (d, J=2.0 Hz 1H), 5.543 (dd, J=4.4 Hz & J=12 Hz, 1H), 3.80 (q, J=7.6 Hz, 1H), 3.14 (dd, J=4.8 Hz & J=18.8 Hz, 1H), 2.64 (m, 2H), 2.55 (s, 3H), 1.04 (t, J=7.2 Hz, 3H), [NH proton is not visible in NMR]; LCMS: m/z 416.10 [M+H]⁺.

### Example-44: Synthesis of 5-(5-(4-ethylphenyl)-1-propionyl-4,5-dihydro-1H-pyrazol-3-yl)-4-methylthieno[2,3-b]pyridin-6(7H)-one

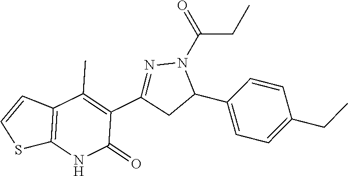 [SMILES: CCC(=O)N1N=C(c2c(C)c3ccsc3[nH]c2=O)CC1c1ccc(CC)cc1]

### Step-a: Synthesis of (E)-5-(3-(4-ethylphenyl)acryloyl)-4-methylthieno[2,3-b]pyridin-6(7H)-one (44a)

The titled compound was synthesized using the same procedure which was followed for (E)-6-Chloro-3-(3-(4-methoxyphenyl)acryloyl)-4-methylquinolin-2(1H)-one (compound-1c) using 5-acetyl-4-methylthieno[2,3-b]pyridin-6(7H)-one and 4-ethylbenzaldehyde as starting materials. Yield: 83%. LCMS (m/z): 324.1 [M+H]⁺.

### Step-b: Synthesis of 5-(5-(4-ethylphenyl)-1-propionyl-4,5-dihydro-1H-pyrazol-3-yl)-4-methylthieno[2,3-b]pyridin-6(7H)-one

The titled compound was synthesized using the same procedure which was followed for 6-chloro-3-(5-(4-methoxyphenyl)-1-propionyl-4,5-dihydro-1H-pyrazol-3-yl)-4-methylquinolin-2(1H)-one (Example-7) using (E)-5-(3-(4-ethylphenyl)acryloyl)-4-methylthieno[2,3-b]pyridin-6(7H)-one as starting material. Yield: 34%, ¹H-NMR (400 MHz, DMSO-d6): [symbol] 7.35-7.33 (m, 2H), 7.17-7.14 (m, 4H), 5.55 (dd, J=4.4 Hz & 12.0 Hz, 1H), 3.78 (dd, J=11.6 Hz & 18.4 Hz, 1H), 3.12 (dd, J=4.4 Hz & 14.0 Hz, 1H), 2.64 (q, J=7.6 Hz, 2H), 2.63-2.59 (m, 2H), 2.54 (s, 3H), 1.17 (t, J=7.2 Hz, 3H), 1.03 (t, J=7.6 Hz, 3H), One exchangeable proton not observed in NMR; LCMS (m/z): 394.1 [M+H]⁺.

### Example-45: Synthesis of 4-methyl-5-(5-(6-methylpyridin-3-yl)-1-propionyl-4,5-dihydro-1H-pyrazol-3-yl)thieno[2,3-b]pyridin-6(7H)-one

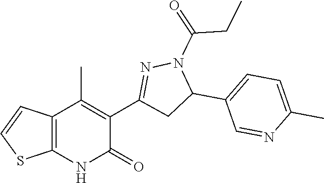 [SMILES: CCC(=O)N1N=C(c2c(C)c3ccsc3[nH]c2=O)CC1c1ccc(C)nc1]

### Step-a: Synthesis of (E)-4-methyl-5-(3-(6-methylpyridin-3-yl)acryloyl)thieno[2,3-b]pyridin-6(7H)-one (45a)

The titled compound was synthesized using the same procedure which was followed for (E)-6-Chloro-3-(3-(4-methoxyphenyl)acryloyl)-4-methylquinolin-2(11H)-one (compound-1c) using 5-acetyl-4-methylthieno[2,3-b]pyridin-6(7H)-one and 6-methylnicotinaldehyde as starting materials. Yield: 98%. LCMS (m/z): 311.1 [M+H]⁺.

### Step-b: Synthesis of 4-methyl-5-(5-(6-methylpyridin-3-yl)-1-propionyl-4,5-dihydro-1H-pyrazol-3-yl)thieno[2,3-b]pyridin-6(7H)-one

The titled compound was synthesized using the same procedure which was followed for 6-chloro-3-(5-(4-methoxyphenyl)-1-propionyl-4,5-dihydro-1H-pyrazol-3-yl)-4-methylquinolin-2(1H)-one (Example-7) using (E)-4-methyl-5-(3-(6-methylpyridin-3-yl)acryloyl)thieno[2,3-b]pyridin-6(7H)-one as starting material. Yield: 22%, 1H-NMR (400 MHz, DMSO-d6): ⊐ 12.50 (bs, 1H), 8.39 (d, J=2.0 Hz, 1H), 7.56 (dd, J=2.0 Hz & 8.0 Hz, 1H), 7.35-7.31 (m, 2H), 7.23 (d, J=8.0 Hz, 1H), 5.52 (dd, J=4.4 Hz & 12.0 Hz, 1H), 3.78 (dd, J=12.0 Hz & 18.4 Hz, 1H), 3.22 (dd, J=4.0 Hz &18.4 Hz, 1H), 2.64 (q, J=7.6 Hz, 2H), 2.58 (s, 3H), 2.44 (s, 3H), 1.02 (t, J=7.2 Hz, 3H); LCMS (m/z): 381.1 [M+H]⁺.

### Chiral Purification of Compound 45.

Step-e: 45 (A): HPLC using a chiral pack IA 5 uM, 4.6*250 mm, IPA (100) eluent at flow rate of 0.7 mL/min at 20° C. The isolated enantiomer at 19.45 min, (the dextro (+) isomer).

Step-f: 45 (B): HPLC using a chiral pack IA 5 uM, 4.6*250 mm, IPA/DCM (90/10) eluent at flow rate of 0.7 mL/min at 20° C. The isolated enantiomer at 41.22 min, (the levo (−) isomer).

### Example-46: Synthesis of 4-methyl-5-(1-propionyl-5-(4-(pyrrolidin-1-yl)phenyl)-4,5-dihydro-1H-pyrazol-3-yl)thieno[2,3-b]pyridin-6(7H)-one

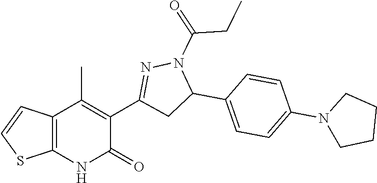 [SMILES: CCC(=O)N1N=C(c2c(C)c3ccsc3[nH]c2=O)CC1c1ccc(N2CCCC2)cc1]

### Step-a: 4-methyl-5(-5-(4-(pyrrolidin-1-yl)phenyl)-4,5-dihydro-1H-pyrazol-3-yl)thieno[2,3-b]pyridin-6(7H)-one

To a solution of (E)-4-methyl-5-(3-(4-(pyrrolidin-1-yl)phenyl)acryloyl)thieno[2,3-b]pyridin-6(7H)-one (155 mg, 0.315 mmol) in ethanol (3 mL) was added 99% hydrazine hydrate (0.1 mL) and refluxed for 2 h. Progress of the reaction was monitored by TLC. The reaction mixture was allowed to cool to room temperature and ethanol was evaporated under reduced pressure. The residue was diluted with ethanol (15 mL) and solid obtained was filtered, washed with water and dried under vacuum to afford the titled compound as off white solid. Yield: 50 mg (42%). LC-MS: m/z 378.49[M+H]⁺.

### Step-b: Synthesis of 4-methyl-5-(1-propionyl-5-(4-(pyrrolidin-1-yl)phenyl)-4,5-dihydro-1H-pyrazol-3-yl)thieno[2,3-b]pyridin-6(7H)-one

To a stirred solution of 4-methyl-5(-5-(4-(pyrrolidin-1-yl)phenyl)-4,5-dihydro-1H-pyrazol-3-yl)thieno[2,3-b]pyridin-6(7H)-one (0.05 g, 0.131 mmol) in DCM (3 mL) at 0° C. was added pyridine (0.031 g, 0.394 mmol), DMAP (0.001 g, 0.157 mmol) and propionyl chloride (0.014 g, 0.0131 mmol). The reaction mixture was allowed to warm to room temperature and stirred for 1 h. Progress of the reaction was monitored by TLC. The reaction mixture was extracted with DCM (10 mL) and water (10 mL). The organic layer was washed brine (10 mL), dried over Na₂SO₄ and evaporated under reduced pressure to get crude compound which was purified by flash column chromatography using 3-2% Methanol in DCM as an eluent to afford the titled compound as off-white solid. Yield: 0.012 g (19%); ¹H-NMR (300 MHz, DMSO-d6): □ 7.336 (s, 2H), 7.091 (d, J=8.8 Hz, 2H), 6.478 (d, J=8.8 Hz, 2H), 5.359 (dd, J=3.6 Hz & J=11.6 Hz, 1H), 3.709 (m, 1H), 3.193 (t, J=6.4 Hz, 4H), 3.14-3.08 (dd, J=4 Hz & J=18.4 Hz), 2.67-2.57 (m, 2H), 2.541 (s, 3H), 1.934 (t, J=6.4 Hz, 4H) [NH proton is not visible in NMR]: LCMS: m/z 434.56[M+H]

### Example-47: Synthesis of 5-(5-(4-cyclopropylphenyl)-1-propionyl-4,5-dihydro-1H-pyrazol-3-yl)-4-methylthieno[2,3-b]pyridin-6(7H)-one

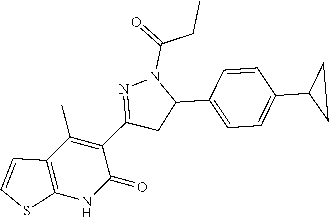 [SMILES: CCC(=O)N1N=C(c2c(C)c3ccsc3[nH]c2=O)CC1c1ccc(C2CC2)cc1]

### Step-a: Synthesis of (E)-5-(3-(4-cyclopropylphenyl)acryloyl)-4-methylthieno[2,3-b]pyridin-6(7H)-one (47a)

The titled compound was synthesized using the same procedure which was followed for (E)-6-Chloro-3-(3-(4-methoxyphenyl)acryloyl)-4-methylquinolin-2(11H)-one (compound-1c) using 5-acetyl-4-methylthieno[2,3-b]pyridin-6(7H)-one and 4-cyclopropylbenzaldehyde as starting materials. Yield: 86%. ¹H-NMR (400 MHz, DMSO-d6): ⊐ 7.56 (d, J=8.4 Hz, 2H), 7.37-7.33 (m, 1H), 7.26 (d, J=5.6 Hz, 2H), 7.11-7.08 (m, 3H), 2.30 (s, 3H), 1.96-1.92 (m, 1H), 1.08-0.96 (m, 2H), 0.73-0.66 (m, 2H), One exchangeable proton not observed in NMR; LCMS (m/z): 336.0 [M+H]⁺.

### Step-b: Synthesis of 5-(5-(4-cyclopropylphenyl)-1-propionyl-4,5-dihydro-1H-pyrazol-3-yl)-4-methylthieno[2,3-b]pyridin-6(7H)-one

The titled compound was synthesized using the same procedure which was followed for 6-chloro-3-(5-(4-methoxyphenyl)-1-propionyl-4,5-dihydro-1H-pyrazol-3-yl)-4-methylquinolin-2(1H)-one (Example-7) using (E)-5-(3-(4-cyclopropylphenyl)acryloyl)-4-methylthieno[2,3-b]pyridin-6(7H)-one as starting material. Yield: 14%, ¹H-NMR (400 MHz, DMSO-d6): ⊐ 7.34-7.33 (m, 2H), 7.13 (d, J=8.0 Hz, 2H), 7.05 (d, J=8.0 Hz, 2H), 5.55 (dd, J=4.4 Hz & 12.0 Hz, 1H), 3.78 (dd, J=11.6 Hz & 18.4 Hz, 1H), 3.12 (dd, J=4.4 Hz & 18.4 Hz, 1H), 2.64 (q, J=7.6 Hz, 2H), 2.54 (s, 3H), 1.90-1.86 (m, 1H), 1.03 (t, J=7.6 Hz, 3H), 0.94-0.89 (m, 2H), 0.66-0.62 (m, 2H), One exchangeable proton not observed in NMR; LCMS (m/z): 406.2 [M+H]⁺.

### Example-48: Synthesis of 4-methyl-5-(5-(2-methylthiazol-4-yl)-1-propionyl-4,5-dihydro-1H-pyrazol-3-yl)thieno[2,3-b]pyridin-6(7H)-one

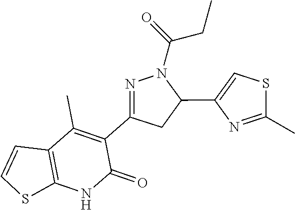 [SMILES: CCC(=O)N1N=C(c2c(C)c3ccsc3[nH]c2=O)CC1c1csc(C)n1]

### Step-a: Synthesis of (E)-4-methyl-5-(3-(2-methylthiazol-4-yl)acryloyl)thieno[2,3-b]pyridin-6(7H)-one

The titled compound was synthesized using the same procedure which was followed for (E)-6-Chloro-3-(3-(4-methoxyphenyl)acryloyl)-4-methylquinolin-2(1H)-one (compound-1c) using 5-acetyl-4-methylthieno[2,3-b]pyridin-6(7H)-one and 2-methylthiazole-4-carbaldehyde as starting materials. Yield: 52%; LCMS: m/z 317.10 [M+H]⁺.

### Step-b: Synthesis of 4-methyl-5-(5-(2-methylthiazol-4-yl)-1-propionyl-4,5-dihydro-1H-pyrazol-3-yl)thieno[2,3-b]pyridin-6(7H)-one)

The titled compound was synthesized using the same procedure which was followed for 6-chloro-3-(5-(4-methoxyphenyl)-1-propionyl-4,5-dihydro-1H-pyrazol-3-yl)-4-methylquinolin-2(1H)-one (Example-7) using (E)-4-methyl-5-(3-(2-methylthiazol-4-yl)acryloyl)thieno[2,3-b]pyridin-6(7H)-one as starting material. Yield: 51%; ¹H-NMR (400 MHz, DMSO-d6): □ 12.45 (bs, H), 7.33 (d, J=5.2 Hz, 2H), 7.23 (s, 1H), 5.57 (dd, J=4.4 Hz & J=11.6 Hz, 1H), 3.76 (q, J=11.6, 1H), 3.16 (dd, J=4.4 Hz & J=18 Hz, 1H), 2.67-2.57 (m, 8H), 1.025 (m, J=14.8 Hz, 3H); LCMS: m/z 387.20[M+H]⁺.

### Example-49: Synthesis of 5-(5-(4-isopropylphenyl)-1-propionyl-4,5-dihydro-1H-pyrazol-3-yl)-4-methylthieno[2,3-b]pyridin-6(7H)-one

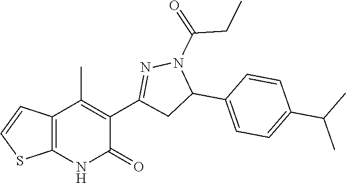 [SMILES: CCC(=O)N1N=C(c2c(C)c3ccsc3[nH]c2=O)CC1c1ccc(C(C)C)cc1]

### Step-a: Synthesis of (E)-5-(3-(4-isopropylphenyl)acryloyl)-4-methylthieno[2,3-b]pyridin-6(7H)-one

The titled compound was synthesized using the same procedure which was followed for (E)-6-Chloro-3-(3-(4-methoxyphenyl)acryloyl)-4-methylquinolin-2(1H)-one (compound-1c) using 5-acetyl-4-methylthieno[2,3-b]pyridin-6(7H)-one and 4-isopropylbenzaldehyde as starting materials. Yield: 61%; LCMS: m/z 338.10[M+H]⁺.

### Step-b: Synthesis of 5-(5-(4-isopropylphenyl)-1-propionyl-4,5-dihydro-1H-pyrazol-3-yl)-4-methylthieno[2,3-b]pyridin-6(7H)-one)

The titled compound was synthesized using the same procedure which was followed for 6-chloro-3-(5-(4-methoxyphenyl)-1-propionyl-4,5-dihydro-1H-pyrazol-3-yl)-4-methylquinolin-2(1H)-one (Example-7) using (E)-5-(3-(4-isopropylphenyl)acryloyl)-4-methylthieno[2,3-b]pyridin-6(7H)-one Synthesis of as starting material. Yield: 20%; ¹H-NMR (400 MHz, DMSO-d6): □ 7.336 (s, 2H), 7.22-7.16 (q, J=8.4 Hz & J=14.8, 4H), 5.443 (dd, J=4.4 Hz & J=12 Hz, 1H), 3.76 (m, J=12 Hz & J=18.4 Hz, 1H), 3.11 (dd, J=4.4 Hz & J=18 Hz, 1H), 2.86 (m, 1H), 2.647 (q, J=7.6 Hz, 2H), 2.55 (s, 3H), 1.19 (d, J=6.8 Hz, 6H), 1.03 (t, J=7.6 Hz, 3H), [NH proton is not visible in NMR]; LCMS: m/z 408.10[M+H]⁺.

### Example-50: Synthesis of 5-(5-(4,6-dimethylpyridin-3-yl)-1-propionyl-4,5-dihydro-1H-pyrazol-3-yl)-4-methylthieno[2,3-b]pyridin-6(7H)-one

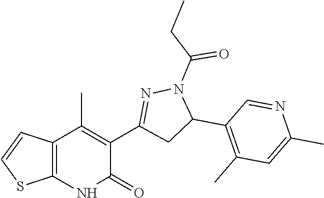 [SMILES: CCC(=O)N1N=C(c2c(C)c3ccsc3[nH]c2=O)CC1c1cnc(C)cc1C]

### Step-a: Synthesis of (E)-5-(3-(4,6-dimethylpyridin-3-yl)acryloyl)-4-methylthieno[2,3-b]pyridin-6(7H)-one

The titled compound was synthesized using the same procedure which was followed for (E)-6-Chloro-3-(3-(4-methoxyphenyl)acryloyl)-4-methylquinolin-2(1H)-one (compound-1c) using 5-acetyl-4-methylthieno[2,3-b]pyridin-6(7H)-one and 4,6-dimethylnicotinaldehyde as starting materials. Yield: 83%; LCMS: m/z 325.0 [M+H]⁺.

### Step-b: Synthesis of 5-(5-(4,6-dimethylpyridin-3-yl)-1-propionyl-4,5-dihydro-1H-pyrazol-3-yl)-4-methylthieno[2,3-b]pyridin-6(7H)-one

The titled compound was synthesized using the same procedure which was followed for 6-chloro-3-(5-(4-methoxyphenyl)-1-propionyl-4,5-dihydro-1H-pyrazol-3-yl)-4-methylquinolin-2(1H)-one (Example-7) using (E)-5-(3-(4,6-dimethylpyridin-3-yl)acryloyl)-4-methylthieno[2,3-b]pyridin-6(7H)-one as starting material. Yield: 36%; ¹H-NMR (400 MHz, DMSO-d6): [symbol] 8.08 (s, 1H), 7.35-7.34 (m, 2H), 7.06 (s, 1H), 5.60 (dd, J=4.8 Hz & J=12.0 Hz, 1H), 3.88 (dd, J=12.0 Hz & J=18.0 Hz, 1H), 3.06 (dd, J=5.2 Hz & J=18.4 Hz, 1H), 2.69 (q, J=7.6 Hz, 2H), 2.58 (s, 3H), 2.38 (s, 3H), 2.33 (s, 3H), 1.05 (t, J=7.2, Hz, 3H), [NH proton is not visible in NMR]; LCMS: m/z 395.10 [M+H]⁺.

### Example-51: Synthesis of 4-methyl-5-(5-(2-methylpyrimidin-5-yl)-1-propionyl-4,5-dihydro-1H-pyrazol-3-yl)thieno[2,3-b]pyridin-6(7H)-one

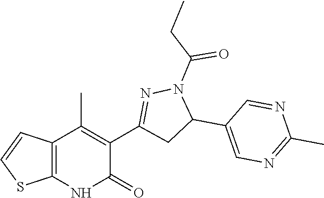 [SMILES: CCC(=O)N1N=C(c2c(C)c3ccsc3[nH]c2=O)CC1c1cnc(C)nc1]

### Step-a: Synthesis of (E)-4-methyl-5-(3-(2-methylpyrimidin-5-yl)acryloyl)thieno[2,3-b]pyridin-6(7H)-one

The titled compound was synthesized using the same procedure which was followed for (E)-6-Chloro-3-(3-(4-methoxyphenyl)acryloyl)-4-methylquinolin-2(1H)-one (compound-1c) using 5-acetyl-4-methylthieno[2,3-b]pyridin-6(7H)-one and 2-methylpyrimidine-5-carbaldehyde as starting materials. Yield: 20%; LCMS: m/z 312.0 [M+H]⁺.

### Step-b: Synthesis of 4-methyl-5-(5-(2-methylpyrimidin-5-yl)-1-propionyl-4,5-dihydro-1H-pyrazol-3-yl)thieno[2,3-b]pyridin-6(7H)-one

The titled compound was synthesized using the same procedure which was followed for 6-chloro-3-(5-(4-methoxyphenyl)-1-propionyl-4,5-dihydro-1H-pyrazol-3-yl)-4-methylquinolin-2(1H)-one (Example-7) using (E)-5-(3-(4,6-dimethylpyridin-3-yl)acryloyl)-4-methylthieno[2,3-b]pyridin-6(7H)-one as starting material. Yield: 50%; ¹H-NMR (400 MHz, DMSO-d6): [symbol] 8.66 (s, 2H), 7.33-725 (m, 2H), 5.54 (dd, J=4.4 Hz & J=11.6 Hz, 1H), 3.76 (dd, J=12.4 Hz & J=18.8 Hz, 1H), 3.28 (m, 1H), 2.60 (q, J=7.6 Hz, 2H), 2.58 (s, 3H), 2.54 (s, 3H), 1.01 (t, J=7.6, Hz, 3H), [NH proton is not visible in NMR]; LCMS: m/z 382.0 [M+H]⁺. Example-52: Synthesis of 5-(5-(5-chloro-6-methylpyridin-3-yl)-1-propionyl-4,5-dihydro-1H-pyrazol-3-yl)-4-methylthieno[2,3-b]pyridin-6(7H)-one

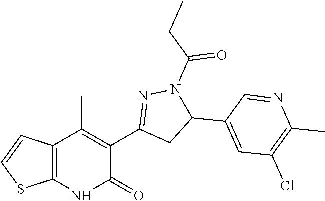 [SMILES: CCC(=O)N1N=C(c2c(C)c3ccsc3[nH]c2=O)CC1c1cnc(C)c(Cl)c1]

### Step-a: Synthesis of (E)-5-(3-(5-chloro-6-methylpyridin-3-yl)acryloyl)-4-methylthieno[2,3-b]pyridin-6(7H)-one

The titled compound was synthesized using the same procedure which was followed for (E)-6-Chloro-3-(3-(4-methoxyphenyl)acryloyl)-4-methylquinolin-2(1H)-one (compound-1c) using 5-acetyl-4-methylthieno[2,3-b]pyridin-6(7H)-one and 5-chloro-6-methylnicotinaldehyde as starting materials. Yield: 99%; LCMS: m/z 344.90 [M+H]⁺.

### Step-b: Synthesis of 5-(5-(5-chloro-6-methylpyridin-3-yl)-1-propionyl-4,5-dihydro-1H-pyrazol-3-yl)-4-methylthieno[2,3-b]pyridin-6(7H)-one

The titled compound was synthesized using the same procedure which was followed for 6-chloro-3-(5-(4-methoxyphenyl)-1-propionyl-4,5-dihydro-1H-pyrazol-3-yl)-4-methylquinolin-2(1H)-one (Example-7) using (E)-5-(3-(5-chloro-6-methylpyridin-3-yl)acryloyl)-4-methylthieno[2,3-b]pyridin-6(7H)-one as starting material. Yield: 10%; ¹H-NMR (400 MHz, DMSO-d6): [symbol] 8.38 (s, 1H), 7.80 (s, 1H), 7.4-7.32 (m, 2H), 5.55 (dd, J=4.4, Hz & J=11.6, Hz, 1H), 3.76 (dd, J=11.6 Hz & J=18.4 Hz, 1H), 3.28 (dd, J=4.8 Hz & J=18.8 Hz, 1H), 2.63 (q, J=7.6 Hz, 2H), 2.54 (s, 3H), 2.52 (s, 3H), 1.01 (t, J=7.6, Hz, 3H), [NH proton is not visible in NMR]; LCMS: m/z 414.90 [M+H]⁺.

### Example-53: Synthesis of 5-(5-(4-methoxy-6-methylpyridin-3-yl)-1-propionyl-4,5-dihydro-1H-pyrazol-3-yl)-4-methylthieno[2,3-b]pyridin-6(7H)-one

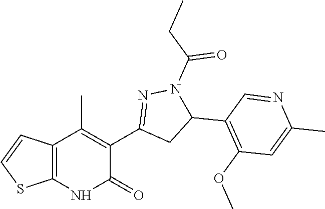 [SMILES: CCC(=O)N1N=C(c2c(C)c3ccsc3[nH]c2=O)CC1c1cnc(C)cc1OC]

### Step-a: Synthesis of (E)-5-(3-(4-methoxy-6-methylpyridin-3-yl)acryloyl)-4-methylthieno[2,3-b]pyridin-6(7H)-one

The titled compound was synthesized using the same procedure which was followed for (E)-6-Chloro-3-(3-(4-methoxyphenyl)acryloyl)-4-methylquinolin-2(1H)-one (compound-1c) using 5-acetyl-4-methylthieno[2,3-b]pyridin-6(7H)-one and 4-methoxy-6-methylnicotinaldehyde as starting materials. Yield: 99%; LCMS: m/z 341.00 [M+H]⁺.

### Step-b: Synthesis of 5-(5-(4-methoxy-6-methylpyridin-3-yl)-1-propionyl-4,5-dihydro-1H-pyrazol-3-yl)-4-methylthieno[2,3-b]pyridin-6(7H)-one

The titled compound was synthesized using the same procedure which was followed for 6-chloro-3-(5-(4-methoxyphenyl)-1-propionyl-4,5-dihydro-1H-pyrazol-3-yl)-4-methylquinolin-2(1H)-one (Example-7) using 5-(5-(4-methoxy-6-methylpyridin-3-yl)-1-propionyl-4,5-dihydro-1H-pyrazol-3-yl)-4-methylthieno[2,3-b]pyridin-6(7H)-one as starting material. Yield: 14%; ¹H-NMR (400 MHz, DMSO-d6): ⊐ 7.92 (s, 1H), 7.34-7.32 (m, 2H), 6.95 (s, 1H), 5.54 (dd, J=4.8, Hz & J=12.0, Hz, 1H), 3.84 (s, 3H), 3.81 (dd, J=11.6 Hz & J=18.4 Hz, 1H), 2.99 (dd, J=4.8 Hz & J=18.8 Hz, 1H), 2.62 (q, J=7.6 Hz, 2H), 2.59 (s, 3H), 2.41 (s, 3H), 1.05 (t, J=7.2, Hz, 3H), [NH proton is not visible in NMR]; LCMS: m/z 411.0 [M+H]⁺.

### Example-54: Synthesis of 5-(5-(5,6-dimethylpyridin-3-yl)-1-propionyl-4,5-dihydro-1H-pyrazol-3-yl)-4-methylthieno[2,3-b]pyridin-6(7H)-one

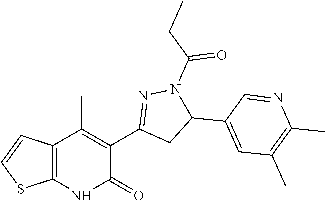 [SMILES: CCC(=O)N1N=C(c2c(C)c3ccsc3[nH]c2=O)CC1c1cnc(C)c(C)c1]

### Step-a: Synthesis of (E)-5-(3-(5,6-dimethylpyridin-3-yl)acryloyl)-4-methylthieno[2,3-b]pyridin-6(7H)-one

The titled compound was synthesized using the same procedure which was followed for (E)-6-Chloro-3-(3-(4-methoxyphenyl)acryloyl)-4-methylquinolin-2(1H)-one (compound-1c) using 5-acetyl-4-methylthieno[2,3-b]pyridin-6(7H)-one and 5,6-dimethylnicotinaldehyde as starting materials. ¹H-NMR (400 MHz, DMSO-d6): [symbol] 12.07 (bs, 1H), 8.52 (s, 1H), 7.95 (s, 1H), 7.41 (d, J=16.0 Hz, 1H), 7.34-7.32 (m, 2H), 7.18 (d, J=16.4 Hz, 1H), 2.45 (s, 3H), 2.35 (m, 3H). 2.26 (s, 3H). Yield: 88%; LCMS: m/z 325.00 [M+H]⁺.

### Step-b: Synthesis of 5-(5-(5,6-dimethylpyridin-3-yl)-1-propionyl-4,5-dihydro-1H-pyrazol-3-yl)-4-methylthieno[2,3-b]pyridin-6(7H)-one

The titled compound was synthesized using the same procedure which was followed for 6-chloro-3-(5-(4-methoxyphenyl)-1-propionyl-4,5-dihydro-1H-pyrazol-3-yl)-4-methylquinolin-2(1H)-one (Example-7) using (E)-5-(3-(5,6-dimethylpyridin-3-yl)acryloyl)-4-methylthieno[2,3-b]pyridin-6(7H)-one as starting material. Yield: 7%; ¹H-NMR (400 MHz, DMSO-d6): [symbol] 8.20 (d, J=2.0, 1H), 7.42 (s, 1H), 7.34-7.32 (m, 2H), 6.95 (s, 1H), 5.54 (dd, J=4.4, Hz & J=12.0, Hz, 1H), 3.79 (dd, J=12.0 Hz & J=18.4 Hz, 1H), 3.20 (dd, J=4.4 Hz & J=18.4 Hz, 1H), 2.62 (q, J=7.2 Hz, 2H), 2.55 (s, 3H), 2.39 (s, 3H), 2.23 (s, 3H), 1.05 (t, J=7.6, Hz, 3H), [NH proton is not visible in NMR]; LCMS: m/z 395.10 [M+H]⁺.

### Example-55: Synthesis of 5-(5-(5-fluoro-6-methylpyridin-3-yl)-1-propionyl-4,5-dihydro-1H-pyrazol-3-yl)-4-methylthieno[2,3-b]pyridin-6(7H)-one

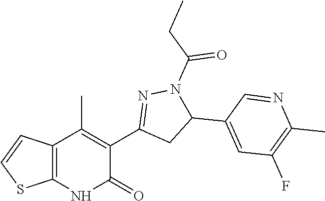 [SMILES: CCC(=O)N1N=C(c2c(C)c3ccsc3[nH]c2=O)CC1c1cnc(C)c(F)c1]

### Step-a: Synthesis of (E)-5-(3-(5-fluoro-6-methylpyridin-3-yl)acryloyl)-4-methylthieno[2,3-b]pyridin-6(7H)-one

The titled compound was synthesized using the same procedure which was followed for (E)-6-Chloro-3-(3-(4-methoxyphenyl)acryloyl)-4-methylquinolin-2(1H)-one (compound-1c) using 5-acetyl-4-methylthieno[2,3-b]pyridin-6(7H)-one and 5-fluoro-6-methylnicotinaldehyde as starting materials. ¹H-NMR (400 MHz, DMSO-d6): ⊐ 12.09 (bs, 1H), 8.61 (s, 1H), 8.12 (d, J=9.6 Hz, 1H), 8.47 (d, J=16.8 Hz, 1H), 7.34-7.32 (m, 2H), 7.27 (d, J=16.4 Hz, 1H), 2.47 (s, 3H), 2.34 (s, 3H). Yield: 63%; LCMS: m/z 328.90 [M+H]⁺.

### Step-b: Synthesis of 5-(5-(5-fluoro-6-methylpyridin-3-yl)-1-propionyl-4,5-dihydro-1H-pyrazol-3-yl)-4-methylthieno[2,3-b]pyridin-6(7H)-one

The titled compound was synthesized using the same procedure which was followed for 6-chloro-3-(5-(4-methoxyphenyl)-1-propionyl-4,5-dihydro-1H-pyrazol-3-yl)-4-methylquinolin-2(1H)-one (Example-7) using (E)-5-(3-(5-fluoro-6-methylpyridin-3-yl)acryloyl)-4-methylthieno[2,3-b]pyridin-6(7H)-one as starting material. Yield: 8%; ¹H-NMR (400 MHz, DMSO-d6): [symbol] 8.29 (d, J=2.0, 1H), 7.56 (d, J=9.2, 1H), 7.35-7.32 (m, 2H), 5.56 (dd, J=4.4, Hz & J=11.6, Hz, 1H), 3.77 (dd, J=11.6 Hz & J=18.4 Hz, 1H), 3.26 (dd, J=4.4 Hz & J=18.4 Hz, 1H), 2.62 (q, J=7.2 Hz, 2H), 2.59 (s, 3H), 2.49 (s, 3H), 1.02 (t, J=7.6, Hz, 3H), [NH proton is not visible in NMR]; LCMS: m/z 399.00 [M+H]⁺.

### Example-56: Synthesis of 5-(5-(4-chloro-6-methylpyridin-3-yl)-1-propionyl-4,5-dihydro-1H-pyrazol-3-yl)-4-methylthieno[2,3-b]pyridin-6(7H)-one

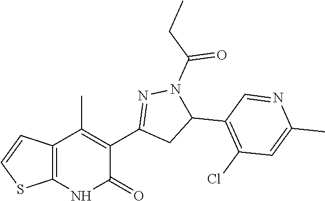 [SMILES: CCC(=O)N1N=C(c2c(C)c3ccsc3[nH]c2=O)CC1c1cnc(C)cc1Cl]

### Step-a: Synthesis of (E)-5-(3-(4-chloro-6-methylpyridin-3-yl)acryloyl)-4-methylthieno[2,3-b]pyridin-6(7H)-one

To a stirred solution of 5-acetyl-4-methylthieno[2,3-b]pyridin-6(7H)-one (0.3 g, 1.44 mmol) in Ethanol (10 mL) was added 4-chloro-6-methylnicotinaldehyde (0.34 g, 2.17 mmol) and the reaction mixture was cooled to 0° C. and then 25% aq. sodium hydroxide (1.15 mL) was added slowly in drop-wise manner for 3 to 5 min. The reaction mixture was allowed to stir at room temperature for 1 h and progress of the reaction was monitored by TLC. The reaction mixture was cooled to 0° C. and acidified with 6 N HCl (up to pH˜5) and extracted with ethyl acetate (3×50 mL). The combined organic layer was washed with brine (25 mL), dried over Na₂SO₄ and evaporated under reduced pressure to get the crude compound which was purified by flash column chromatography using 50-60% ethyl acetate in hexane as an eluent to afford the titled compound as off-white solid. Yield: 0.48 g (96%); LCMS: m/z 345.00 [M+H]⁺.

### Step-b: Synthesis of 5-(5-(4-chloro-6-methylpyridin-3-yl)-4,5-dihydro-1H-pyrazol-3-yl)-4-methylthieno[2,3-b]pyridin-6(7H)-one

The titled compound was synthesized using the same procedure which was followed for 6-Chloro-3-(5-(4-methoxyphenyl)-4,5-dihydro-1H-pyrazol-3-yl)-4-methylquinolin-2(1H)-one (Example-2a) using (E)-5-(3-(4-chloro-6-methylpyridin-3-yl)acryloyl)-4-methylthieno[2,3-b]pyridin-6(7H)-one as starting material. Yield: 44%; LCMS: m/z 358.90 [M+H]⁺.

### Step-c: Synthesis of 5-(5-(4-chloro-6-methylpyridin-3-yl)-1-propionyl-4,5-dihydro-1H-pyrazol-3-yl)-4-methylthieno[2,3-b]pyridin-6(7H)-one

The titled compound was synthesized using the same procedure which was followed for 6-Chloro-3-(1-(cyclopropanecarbonyl)-5-(4-methoxyphenyl)-4,5-dihydro-1H-pyrazol-3-yl)-4-methylquinolin-2(1H)-one (Example-2) using 5-(5-(4-chloro-6-methylpyridin-3-yl)-4,5-dihydro-1H-pyrazol-3-yl)-4-methylthieno[2,3-b]pyridin-6(7H)-one as starting material. Yield: 47%; ¹H-NMR (400 MHz, DMSO-d6): [symbol] 8.19 (s, 1H), 7.46 (s, 1H), 7.34-7.32 (m, 2H), 5.69 (dd, J=5.2, Hz & J=11.6, Hz, 1H), 3.91 (dd, J=12.0 Hz & J=18.4 Hz, 1H), 3.13 (dd, J=5.6 Hz & J=18.4 Hz, 1H), 2.66 (q, J=7.2 Hz, 2H), 2.57 (s, 3H), 2.45 (s, 3H), 1.06 (t, J=7.2, Hz, 3H), [NH proton is not visible in NMR]; LCMS: m/z 414.90 [M+H]⁺.

### Example-57: Synthesis of 4-methyl-5-(5-(2-methylthiazol-5-yl)-1-propionyl-4,5-dihydro-1H-pyrazol-3-yl)thieno[2,3-b]pyridin-6(7H)-one

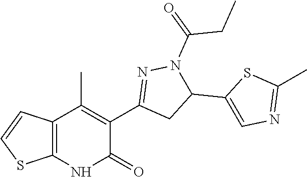 [SMILES: CCC(=O)N1N=C(c2c(C)c3ccsc3[nH]c2=O)CC1c1cnc(C)s1]

### Step-a: Synthesis of (E)-4-methyl-5-(3-(2-methylthiazol-5-yl)acryloyl)thieno[2,3-b]pyridin-6(7H)-one

The titled compound was synthesized using the same procedure which was followed for (E)-6-Chloro-3-(3-(4-methoxyphenyl)acryloyl)-4-methylquinolin-2(1H)-one (compound-1c) using 5-acetyl-4-methylthieno[2,3-b]pyridin-6(7H)-one and 2-methylthiazole-5-carbaldehyde as starting materials. Yield: 96%; LCMS: m/z 317.10 [M+H]⁺.

### Step-b: Synthesis of 5-(5-(2-methoxythiazol-5-yl)-1-propionyl-4,5-dihydro-1H-pyrazol-3-yl)-4-methylthieno[2,3-b]pyridin-6(7H)-one

The titled compound was synthesized using the same procedure which was followed for 6-chloro-3-(5-(4-methoxyphenyl)-1-propionyl-4,5-dihydro-1H-pyrazol-3-yl)-4-methylquinolin-2(1H)-one (Example-7) using (E)-4-methyl-5-(3-(2-methylthiazol-5-yl)acryloyl)thieno[2,3-b]pyridin-6(7H)-one as starting material. Yield: 43%; ¹H-NMR (400 MHz, DMSO-d6): ⊐ 7.63 (s, 1H), 7.35-7.32 (m, 2H), 5.81 (dd, J=3.2, Hz & J=11.2, Hz, 1H), 3.79 (dd, J=11.6 Hz & J=18.4 Hz, 1H), 3.39 (dd, J=3.2 Hz & J=18.0 Hz, 1H), 2.59 (q, J=7.2 Hz, 2H), 2.59 (s, 3H), 2.49 (s, 3H), 1.02 (t, J=7.6, Hz, 3H), [NH proton is not visible in NMR]; LCMS: m/z 387.0 [M+H]⁺.

### Example-58: Synthesis of 5-(5-cyclohexyl-1-propionyl-4,5-dihydro-1H-pyrazol-3-yl)-4-methylthieno[2,3-b]pyridin-6(7H)-one

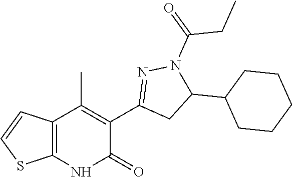 [SMILES: CCC(=O)N1N=C(c2c(C)c3ccsc3[nH]c2=O)CC1C1CCCCC1]

### Step-a: Synthesis of (E)-5-(3-cyclohexylacryloyl)-4-methylthieno[2,3-b]pyridin-6(7H)-one

The titled compound was synthesized using the same procedure which was followed for (E)-6-Chloro-3-(3-(4-methoxyphenyl)acryloyl)-4-methylquinolin-2(1H)-one (compound-1c) using 5-acetyl-4-methylthieno[2,3-b]pyridin-6(7H)-one and cyclohexanecarbaldehyde as starting materials. Yield: 21%; LCMS: m/z 302.00 [M+H]⁺.

### Step-b: Synthesis of 5-(5-cyclohexyl-1-propionyl-4,5-dihydro-1H-pyrazol-3-yl)-4-methylthieno[2,3-b]pyridin-6(7H)-one

The titled compound was synthesized using the same procedure which was followed for 6-chloro-3-(5-(4-methoxyphenyl)-1-propionyl-4,5-dihydro-1H-pyrazol-3-yl)-4-methylquinolin-2(1H)-one (Example-7) using (E)-5-(3-cyclohexylacryloyl)-4-methylthieno[2,3-b]pyridin-6(7H)-one as starting material. Yield: 11%; ¹H-NMR (400 MHz, DMSO-d6): [symbol] 7.31-7.29 (m, 2H), 4.40 (dd, J=3.2, Hz & J=6.8, Hz, 1H), 3.26 (dd, J=11.6 Hz & J=18.4 Hz, 1H), 3.10 (dd, J=4.4 Hz & J=18.4 Hz, 1H), 2.62 (q, J=7.2 Hz, 2H), 2.05-2.04 (m, 1H), 1.69-1.51 (m, 4H), 1.36-1.32 (m, 1H), 1.23-1.21 (m, 2H), 1.18-1.14 (m, 2H), 1.08 (t, J=7.6, Hz, 3H), 0.98-0.96 (m, 1H), [NH proton is not visible in NMR]; LCMS: m/z 372.10 [M+H]⁺.

### Example-59: Synthesis of 6-chloro-4-methyl-3-(1-propionyl-5-(p-tolyl)-4,5-dihydro-1H-pyrazol-3-yl)quinolin-2(1H)-one

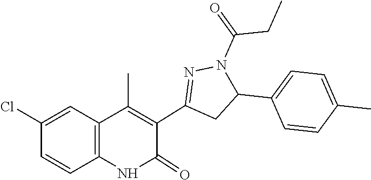 [SMILES: CCC(=O)N1N=C(c2c(C)c3cc(Cl)ccc3[nH]c2=O)CC1c1ccc(C)cc1]

### Step-a: Synthesis of (E)-6-chloro-4-methyl-3-(3-(p-tolyl)acryloyl)quinolin-2(1H)-one

The titled compound was synthesized using the same procedure which was followed for (E)-6-Chloro-3-(3-(4-methoxyphenyl)acryloyl)-4-methylquinolin-2(1H)-one (compound-1c) using 3-acetyl-6-chloro-4-methylquinolin-2(1H)-one and 4-methylbenzaldehyde as starting materials. ¹H-NMR (400 MHz, DMSO-d6): [symbol] 12.09 (bs, 1H), 7.86 (d, J=2.0 Hz, 1H), 7.64-7.62 (m, 3H), 7.46 (d, J=16.8 Hz, 1H), 7.38 (d, J=8.8 Hz, 1H), 7.24 (d, J=8.0 Hz, 2H), 7.03 (d, J=16.0 Hz, 1H), 2.47 (s, 3H), 2.33 (s, 3H), 2.30 (s, 3H) Yield: 83%; LCMS: m/z 338.00 [M+H]⁺.

### Step-b: Synthesis of 6-chloro-4-methyl-3-(1-propionyl-5-(p-tolyl)-4,5-dihydro-1H-pyrazol-3-yl)quinolin-2(1H)-one

The titled compound was synthesized using the same procedure which was followed for 6-chloro-3-(5-(4-methoxyphenyl)-1-propionyl-4,5-dihydro-1H-pyrazol-3-yl)-4-methylquinolin-2(1H)-one (Example-7) using (E)-6-chloro-4-methyl-3-(3-(p-tolyl)acryloyl)quinolin-2(1H)-one as starting material. Yield: 11%; ¹H-NMR (400 MHz, DMSO-d6): ⊐ 12.07 (bs, 1H), 7.89 (d, J=2.0, 1H), 7.62 (dd, J=2.4, Hz & J=8.8, Hz, 1H), 7.37 (d, J=8.8, 1H), 7.18-7.93 (m, 4H), 5.49 (dd, J=4.4, Hz & J=12.0, Hz, 1H), 3.78 (dd, J=12.4 Hz & J=18.8 Hz, 1H), 3.09 (dd, J=4.8 Hz & J=18.4 Hz, 1H), 2.62 (q, J=7.6 Hz, 2H), 2.59 (s, 3H), 2.28 (s, 3H), 1.03 (t, J=7.2, Hz, 3H), LCMS: m/z 408.20 [M+H]⁺.

### Example-60: Synthesis of 4-methyl-3-(1-propionyl-5-(p-tolyl)-4,5-dihydro-1H-pyrazol-3-yl)quinolin-2(1H)-one

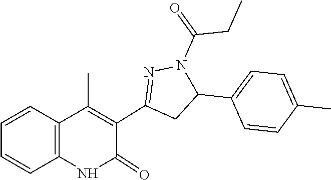 [SMILES: CCC(=O)N1N=C(c2c(C)c3ccccc3[nH]c2=O)CC1c1ccc(C)cc1]

### Step-a: Synthesis of (E)-4-methyl-3-(3-(p-tolyl)acryloyl)quinolin-2(1H)-one

The titled compound was synthesized using the same procedure which was followed for (E)-6-Chloro-3-(3-(4-methoxyphenyl)acryloyl)-4-methylquinolin-2(1H)-one (compound-1c) using 3-acetyl-4-methylquinolin-2(1H)-one and 4-methylbenzaldehyde as starting materials. Yield: 89%; ¹H-NMR (400 MHz, DMSO-d6): [symbol] 11.95 (bs, 1H), 7.83 (d, J=8.4, 1H), 7.69 (d, J=8.0, Hz, 1H), 7.58 (t, J=7.2, Hz, 2H), 7.43 (d, J=5.6, Hz, 1H), 7.37 (d, J=8.4, Hz, 1H), 7.28-7.22 (m, 3H), 7.02 (d, J=16.4, Hz, 1H), 2.35 (s, 3H), 2.32 (s, 3H), LCMS: m/z 304.20 [M+H]⁺

### Step-b: Synthesis of 4-methyl-3-(1-propionyl-5-(p-tolyl)-4,5-dihydro-1H-pyrazol-3-yl)quinolin-2(1H)-one

The titled compound was synthesized using the same procedure which was followed for 6-chloro-3-(5-(4-methoxyphenyl)-1-propionyl-4,5-dihydro-1H-pyrazol-3-yl)-4-methylquinolin-2(1H)-one (Example-7) using (E)-4-methyl-3-(3-(p-tolyl)acryloyl)quinolin-2(1H)-one as starting material. Yield: 37%; ¹H-NMR (400 MHz, DMSO-d6): ⊐ 12.07 (bs, 1H), 7.85 (d, J=8.0, Hz, 1H), 7.56 (t, J=7.2, Hz, 1H), 7.35 (d, J=8.0, Hz, 1H), 7.26 (t, J=7.2, Hz, 1H), 7.19-7.14 (m, 4H), 5.48 (dd, J=4.4, Hz & J=12.0, Hz, 1H), 3.78 (dd, J=12.4 Hz & J=18.4 Hz, 1H), 3.09 (dd, J=4.8 Hz & J=18.4 Hz, 1H), 2.62 (q, J=7.2 Hz, 2H), 2.59 (s, 3H), 2.28 (s, 3H), 1.03 (t, J=7.2, Hz, 3H), LCMS: m/z 374.20 [M+H]⁺.

Chiral purification of compound 60.

Step-e: 60 (A): HPLC using a chiral pack IA 5 uM, 4.6*250 mm, IPA (100) eluent at flow rate of 0.7 mL/min at 20° C. The isolated enantiomer at 20.97 min.

Step-f: 60 (B): HPLC using a chiral pack IA 5 uM, 4.6*250 mm, IPA/DCM (90/10) eluent at flow rate of 0.7 mL/min at 20° C. The isolated enantiomer at 31.04 min.

### Example-61: Synthesis of 6-chloro-4-methyl-3-(1-propionyl-5-(p-tolyl)-4,5-dihydro-1H-pyrazol-3-yl)-1,5-naphthyridin-2(1H)-one

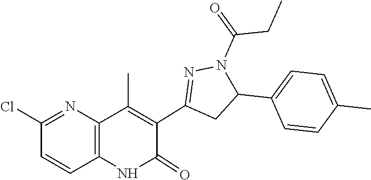 [SMILES: CCC(=O)N1N=C(c2c(C)c3nc(Cl)ccc3[nH]c2=O)CC1c1ccc(C)cc1]

### Step-a: Synthesis of (E)-6-chloro-4-methyl-3-(3-(p-tolyl)acryloyl)-1,5-naphthyridin-2(1H)-one

The titled compound was synthesized using the same procedure which was followed for (E)-6-Chloro-3-(3-(4-methoxyphenyl)acryloyl)-4-methylquinolin-2(1H)-one (compound-1c) using 3-acetyl-6-chloro-4-methyl-1,5-naphthyridin-2(1H)-one and 4-methylbenzaldehyde as starting materials. Yield: 88%; ¹H-NMR (400 MHz, DMSO-d6): ⊐ 12.23 (bs, 1H), 7.78 (d, J=8.8, 1H), 7.68 (d, J=2.8, Hz, 1H), 7.65 (d, J=8.0, Hz, 2H), 7.54 (d, J=16.4, Hz, 1H), 7.25 (d, J=8.0, Hz, 2H), 7.03 (d, J=16.4, Hz, 1H), 2.34 (s, 3H), 2.31 (s, 3H), LCMS: m/z 339.00 [M+H]⁺.

### Step-b: Synthesis of 6-chloro-4-methyl-3-(1-propionyl-5-(p-tolyl)-4,5-dihydro-1H-pyrazol-3-yl)-1,5-naphthyridin-2(1H)-one

The titled compound was synthesized using the same procedure which was followed for 6-chloro-3-(5-(4-methoxyphenyl)-1-propionyl-4,5-dihydro-1H-pyrazol-3-yl)-4-methylquinolin-2(1H)-one (Example-7) using (E)-6-chloro-4-methyl-3-(3-(p-tolyl)acryloyl)-1,5-naphthyridin-2(1H)-one as starting material. Yield: 34%; ¹H-NMR (400 MHz, DMSO-d6): [symbol] 12.23 (bs, 1H), 7.75 (d, J=8.4, Hz, 1H), 7.56 (t, J=8.4, Hz, 1H), 7.16-7.14 (m, 4H), 5.48 (dd, J=4.4, Hz & J=11.6, Hz, 1H), 3.85 (dd, J=12.0 Hz & J=18.4 Hz, 1H), 3.14 (dd, J=4.4 Hz & J=18.4 Hz, 1H), 2.62 (q, J=7.2 Hz, 2H), 2.60 (s, 3H), 2.28 (s, 3H), 1.03 (t, J=7.2, Hz, 3H), LCMS: m/z 409.20 [M+H]⁺.

### Example-62: Synthesis of 5-(1-acetyl-5-(p-tolyl)-4,5-dihydro-1H-pyrazol-3-yl)-4-methyl-7,7a-dihydrothieno[2,3-b]pyridin-6(7H)-one

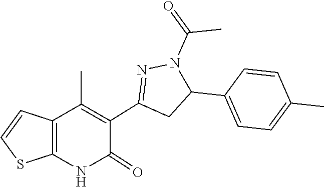 [SMILES: CC(=O)N1N=C(c2c(C)c3ccsc3[nH]c2=O)CC1c1ccc(C)cc1]

### Step-a: Synthesis of 4-methyl-5-(5-(p-tolyl)-4,5-dihydro-1H-pyrazol-3-yl)-7,7a-dihydrothieno[2,3-b]pyridin-6(7H)-one

The titled compound was synthesized using the same procedure which was followed for 6-Chloro-3-(5-(4-methoxyphenyl)-4,5-dihydro-1H-pyrazol-3-yl)-4-methylquinolin-2(1H)-one (compound-2a) using (E)-4-methyl-5-(3-(p-tolyl)acryloyl)-7,7a-dihydrothieno[2,3-b]pyridin-6(3aH)-one and hydrazine hydrate as starting materials. Yield: 72%; LCMS: m/z 324.10 [M+H]⁺.

### Step-b: Synthesis of 5-(1-acetyl-5-(p-tolyl)-4,5-dihydro-1H-pyrazol-3-yl)-4-methyl-7,7a-dihydrothieno[2,3-b]pyridin-6(7H)-one

The titled compound was synthesized using the same procedure which was followed for 6-Chloro-3-(1-(cyclopropanecarbonyl)-5-(4-methoxyphenyl)-4,5-dihydro-1H-pyrazol-3-yl)-4-methylquinolin-2(11H)-one (Example-2) using 4-methyl-5-(5-(p-tolyl)-4,5-dihydro-1H-pyrazol-3-yl)-7,7a-dihydrothieno[2,3-b]pyridin-6(3aH)-one and acetyl chloride as starting material. Yield: 53%; ¹H-NMR (400 MHz, DMSO-d6): [symbol] 7.34-7.32 (m, 2H), 7.18-7.13 (m, 4H), 5.46 (dd, J=4.0, Hz & J=11.6, Hz, 1H), 3.79 (dd, J=11.6 Hz & J=18.4 Hz, 1H), 3.13 (dd, J=4.0 Hz & J=18.0 Hz, 1H), 2.54 (s, 3H), 2.27 (s, 3H), 2.21 (s, 3H), [NH proton is not visible in NMR]; LCMS: m/z 366.00 [M+H]⁺.

### Example-63: Synthesis of 5-(1-isobutyryl-5-(p-tolyl)-4,5-dihydro-1H-pyrazol-3-yl)-4-methyl-7,7a-dihydrothieno[2,3-b]pyridin-6(7H)-one

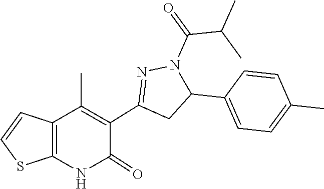 [SMILES: Cc1ccc(C2CC(c3c(C)c4ccsc4[nH]c3=O)=NN2C(=O)C(C)C)cc1]

### Step-a: Synthesis of 5-(1-isobutyryl-5-(p-tolyl)-4,5-dihydro-1H-pyrazol-3-yl)-4-methyl-7,7a-dihydrothieno[2,3-b]pyridin-6(7H)-one

The titled compound was synthesized using the same procedure which was followed for 6-Chloro-3-(1-(cyclopropanecarbonyl)-5-(4-methoxyphenyl)-4,5-dihydro-1H-pyrazol-3-yl)-4-methylquinolin-2(11H)-one (Example-2) using 4-methyl-5-(5-(p-tolyl)-4,5-dihydro-1H-pyrazol-3-yl)-7,7a-dihydrothieno[2,3-b]pyridin-6(3aH)-one and isobutyryl chloride as starting material. Yield: 45%; ¹H-NMR (400 MHz, DMSO-d6): [symbol] 7.34-7.32 (m, 2H), 7.18-7.13 (m, 4H), 5.46 (dd, J=4.4, Hz & J=12.0, Hz, 1H), 3.79 (dd, J=12.0 Hz & J=18.4 Hz, 1H), 3.32-3.27 (m, 1H), 3.13 (dd, J=4.4 Hz & J=18.4 Hz, 1H), 2.54 (s, 3H), 2.27 (s, 3H), 1.07-1.04 (m, 6H), [NH proton is not visible in NMR]; LCMS: m/z 394.20 [M+H]⁺.

### Example-64: Synthesis of 4-methyl-5-(5-(p-tolyl)-1-(2,2,2-trifluoroacetyl)-4,5-dihydro-1H-pyrazol-3-yl)-7,7a-dihydrothieno[2,3-b]pyridin-6(7H)-one

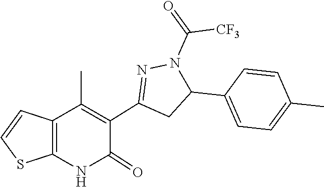 [SMILES: Cc1ccc(C2CC(c3c(C)c4ccsc4[nH]c3=O)=NN2C(=O)C(F)(F)F)cc1]

### Step-a: Synthesis of 4-methyl-5-(5-(p-tolyl)-1-(2,2,2-trifluoroacetyl)-4,5-dihydro-1H-pyrazol-3-yl)-7,7a-dihydrothieno[2,3-b]pyridin-6(7H)-one

The titled compound was synthesized using the same procedure which was followed for 6-Chloro-3-(1-(cyclopropanecarbonyl)-5-(4-methoxyphenyl)-4,5-dihydro-1H-pyrazol-3-yl)-4-methylquinolin-2(11H)-one (Example-2) using 4-methyl-5-(5-(p-tolyl)-4,5-dihydro-1H-pyrazol-3-yl)-7,7a-dihydrothieno[2,3-b]pyridin-6(3aH)-one and 2,2,2-trifluoroacetic anhydride as starting material. Yield: 54%; ¹H-NMR (400 MHz, DMSO-d6): [symbol] 7.37-7.35 (m, 2H), 7.23-7.18 (m, 4H), 5.63 (dd, J=4.0, Hz & J=11.6, Hz, 1H), 3.77 (dd, J=11.6 Hz & J=18.8 Hz, 1H), 3.39 (dd, J=4.4 Hz & J=18.4 Hz, 1H), 2.55 (s, 3H), 2.29 (s, 3H), [NH proton is not visible in NMR]; LCMS: m/z 420.10 [M+H]⁺.

### Example-65: Synthesis of 5-(5-(2-chloro-4-methylphenyl)-1-propionyl-4,5-dihydro-1H-pyrazol-3-yl)-4-methylthieno[2,3-b]pyridin-6(7H)-one

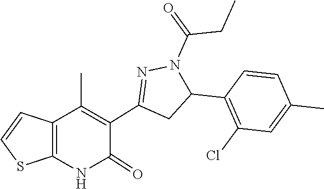 [SMILES: CCC(=O)N1N=C(c2c(C)c3ccsc3[nH]c2=O)CC1c1ccc(C)cc1Cl]

### Step-a: Synthesis of (E)-5-(3-(2-chloro-4-methylphenyl)acryloyl)-4-methylthieno[2,3-b]pyridin-6(7H)-one

The titled compound was synthesized using the same procedure which was followed for (E)-6-Chloro-3-(3-(4-methoxyphenyl)acryloyl)-4-methylquinolin-2(1H)-one (compound-1c) using 5-acetyl-4-methylthieno[2,3-b]pyridin-6(7H)-one and 2-chloro-4-methylbenzaldehyde as starting materials. Yield: 73%; ¹H-NMR (400 MHz, DMSO-d6): ⊐ 12.23 (bs, 1H), 7.83 (d, J=15.6, 1H), 7.45 (d, J=7.2, Hz, 1H), 7.10 (s, 1H), 701.-6.99 (m, 2H), 6.90 (d, J=7.6, Hz, 1H), 6.86 (d, J=5.6, Hz, 1H), 2.37 (s, 3H), 2.26 (s, 3H), LCMS: m/z 344.00 [M+H]⁺.

### Step-b: Synthesis of 5-(5-(2-chloro-4-methylphenyl)-1-propionyl-4,5-dihydro-1H-pyrazol-3-yl)-4-methylthieno[2,3-b]pyridin-6(7H)-one

The titled compound was synthesized using the same procedure which was followed for 6-chloro-3-(5-(4-methoxyphenyl)-1-propionyl-4,5-dihydro-1H-pyrazol-3-yl)-4-methylquinolin-2(1H)-one (Example-7) using (E)-5-(3-(2-chloro-4-methylphenyl)acryloyl)-4-methylthieno[2,3-b]pyridin-6(7H)-one as starting material. Yield: 33%; ¹H-NMR (400 MHz, DMSO-d6): [symbol] 7.35-7.32 (m, 2H), 7.31 (s, 1H), 7.13 (d, J=8.0, Hz, 1H), 7.00 (d, J=8.0, Hz, 1H), 5.67 (dd, J=4.8 Hz & J=12.0, Hz, 1H), 3.87 (dd, J=12.0 Hz & J=18.0 Hz, 1H), 3.03 (dd, J=4.8 Hz & J=18.4 Hz, 1H), 2.72 (q, J=7.2 Hz, 2H), 2.59 (s, 3H), 2.49 (s, 3H), 1.02 (t, J=7.6, Hz, 3H), [NH proton is not visible in NMR]; LCMS: m/z 414.00 [M+H]⁺.

### Example-66: Synthesis of 5-(5-(2-fluoro-4-methylphenyl)-1-propionyl-4,5-dihydro-1H-pyrazol-3-yl)-4-methylthieno[2,3-b]pyridin-6(7H)-one

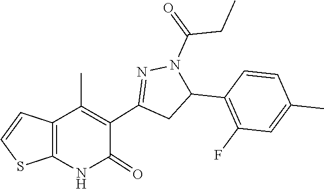 [SMILES: CCC(=O)N1N=C(c2c(C)c3ccsc3[nH]c2=O)CC1c1ccc(C)cc1F]

### Step-a: Synthesis of (E)-5-(3-(2-fluoro-4-methylphenyl)acryloyl)-4-methylthieno[2,3-b]pyridin-6(7H)-one

The titled compound was synthesized using the same procedure which was followed for (E)-6-Chloro-3-(3-(4-methoxyphenyl)acryloyl)-4-methylquinolin-2(1H)-one (compound-1c) using 5-acetyl-4-methylthieno[2,3-b]pyridin-6(7H)-one and 2-fluoro-4-methylbenzaldehyde as starting materials. Yield: 51%; LCMS: m/z 328.00 [M+H]⁺.

### Step-b: Synthesis of 5-(5-(2-fluoro-4-methylphenyl)-1-propionyl-4,5-dihydro-1H-pyrazol-3-yl)-4-methylthieno[2,3-b]pyridin-6(7H)-one

The titled compound was synthesized using the same procedure which was followed for 6-chloro-3-(5-(4-methoxyphenyl)-1-propionyl-4,5-dihydro-1H-pyrazol-3-yl)-4-methylquinolin-2(1H)-one (Example-7) using (E)-5-(3-(2-fluoro-4-methylphenyl)acryloyl)-4-methylthieno[2,3-b]pyridin-6(7H)-one as starting material. Yield: 53%; ¹H-NMR (400 MHz, DMSO-d6): [symbol] 7.35-7.32 (m, 2H), 7.09-6.97 (m, 3H), 5.59 (dd, J=4.8, Hz & J=12.0, Hz, 1H), 3.87 (dd, J=12.4 Hz & J=18.4 Hz, 1H), 3.26 (dd, J=4.8 Hz & J=18.4 Hz, 1H), 2.65 (q, J=7.6 Hz, 2H), 2.59 (s, 3H), 2.29 (s, 3H), 1.05 (t, J=7.6, Hz, 3H), [NH proton is not visible in NMR]; LCMS: m/z 398.20 [M+H]⁺.

### Chiral Purification of Compound 66.

Step-e: 66 (A): HPLC using a chiral pack IA 5 uM, 4.6*250 mm, IPA (100) eluent at flow rate of 0.7 mL/min at 20° C. The isolated enantiomer at 12.43 min.

Step-f: 66 (B): HPLC using a chiral pack IA 5 uM, 4.6*250 mm, IPA/DCM (90/10) eluent at flow rate of 0.7 mL/min at 20° C. The isolated enantiomer at 23.59 min.

### Example-67: Synthesis of 6-chloro-4-methyl-3-(1-propionyl-5-(p-tolyl)-4,5-dihydro-1H-pyrazol-3-yl)-1,8-naphthyridin-2(1H)-one

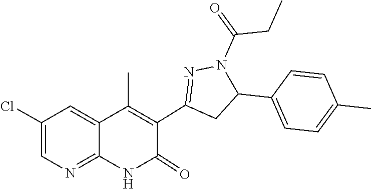 [SMILES: CCC(=O)N1N=C(c2c(C)c3cc(Cl)cnc3[nH]c2=O)CC1c1ccc(C)cc1]

### Step-a: Synthesis of (E)-6-chloro-4-methyl-3-(3-(p-tolyl)acryloyl)-1,8-naphthyridin-2(1H)-one

The titled compound was synthesized using the same procedure which was followed for (E)-6-Chloro-3-(3-(4-methoxyphenyl)acryloyl)-4-methylquinolin-2(1H)-one (compound-1c) using 3-acetyl-6-chloro-4-methyl-1,8-naphthyridin-2(1H)-one and 4-methylbenzaldehyde as starting materials. Yield: 90%; LCMS: m/z 339.10 [M+H]⁺.

### Step-b: Synthesis of 6-chloro-4-methyl-3-(1-propionyl-5-(p-tolyl)-4,5-dihydro-1H-pyrazol-3-yl)-1,8-naphthyridin-2(1H)-one

The titled compound was synthesized using the same procedure which was followed for 6-chloro-3-(5-(4-methoxyphenyl)-1-propionyl-4,5-dihydro-1H-pyrazol-3-yl)-4-methylquinolin-2(1H)-one (Example-7) using (E)-6-chloro-4-methyl-3-(3-(p-tolyl)acryloyl)-1,8-naphthyridin-2(1H)-one as starting material. Yield: 43%; ¹H-NMR (400 MHz, DMSO-d6): ⊐ 12.53 (bs, 1H), 8.61 (d, J=2.4, Hz, 1H), 8.40 (d, J=2.0, Hz, 1H), 7.19-7.13 (m, 4H), 5.49 (dd, J=4.4, Hz & J=11.6, Hz, 1H), 3.82 (dd, J=11.6 Hz & J=18.4 Hz, 1H), 3.14 (dd, J=4.8 Hz & J=18.4 Hz, 1H), 2.64 (q, J=7.2 Hz, 2H), 2.58 (s, 3H), 2.28 (s, 3H), 1.03 (t, J=7.6, Hz, 3H), LCMS: m/z 409.10 [M+H]⁺.

### Example-68: Synthesis of 5-(5-(4-(fluoromethyl)phenyl)-1-propionyl-4,5-dihydro-1H-pyrazol-3-yl)-4-methylthieno[2,3-b]pyridin-6(7H)-one

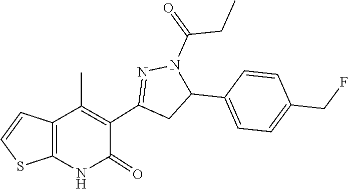 [SMILES: CCC(=O)N1N=C(c2c(C)c3ccsc3[nH]c2=O)CC1c1ccc(CF)cc1]

### Step-a: Synthesis of (E)-5-(3-(4-(fluoromethyl)phenyl)acryloyl)-4-methylthieno[2,3-b]pyridin-6(7H)-one

The titled compound was synthesized using the same procedure which was followed for (E)-6-Chloro-3-(3-(4-methoxyphenyl)acryloyl)-4-methylquinolin-2(1H)-one (compound-1c) using 5-acetyl-4-methylthieno[2,3-b]pyridin-6(7H)-one and 4-(fluoromethyl)benzaldehyde as starting materials. Yield: 76%; LCMS: m/z 328.00 [M+H]⁺.

### Step-b: Synthesis of 5-(5-(4-(fluoromethyl)phenyl)-1-propionyl-4,5-dihydro-1H-pyrazol-3-yl)-4-methylthieno[2,3-b]pyridin-6(7H)-one

The titled compound was synthesized using the same procedure which was followed for 6-chloro-3-(5-(4-methoxyphenyl)-1-propionyl-4,5-dihydro-1H-pyrazol-3-yl)-4-methylquinolin-2(1H)-one (Example-7) using (E)-5-(3-(4-(fluoromethyl)phenyl)acryloyl)-4-methylthieno[2,3-b]pyridin-6(7H)-one as starting material. Yield: 34%; ¹H-NMR (400 MHz, DMSO-d6): [symbol] 7.40-7.38 (m, 2H), 7.33-7.29 (m, 4H), 5.52 (dd, J=4.0, Hz & J=11.6, Hz, 1H), 5.45 (s, 1H), 5.33 (s, 1H), 3.83 (dd, J=12.0 Hz & J=18.4 Hz, 1H), 3.16 (dd, J=4.8 Hz & J=18.4 Hz, 1H), 2.65 (q, J=7.6 Hz, 2H), 2.58 (s, 3H), 2.29 (s, 3H), 1.05 (t, J=7.6, Hz, 3H), [NH proton is not visible in NMR]; LCMS: m/z 398.20 [M+H]⁺.

### Example-69: Synthesis of 4-methyl-3-(1-propionyl-5-(p-tolyl)-4,5-dihydro-1H-pyrazol-3-yl)-1,5-naphthyridin-2(1H)-one

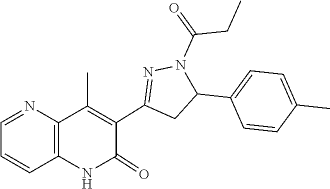 [SMILES: CCC(=O)N1N=C(c2c(C)c3ncccc3[nH]c2=O)CC1c1ccc(C)cc1]

### Step-a: Synthesis of (E)-4-methyl-3-(3-(p-tolyl)acryloyl)-1,5-naphthyridin-2(1H)-one

The titled compound was synthesized using the same procedure which was followed for (E)-6-Chloro-3-(3-(4-methoxyphenyl)acryloyl)-4-methylquinolin-2(1H)-one (compound-1c) using 3-acetyl-4-methyl-1,5-naphthyridin-2(1H)-one as starting materials. Yield: 54%; LCMS: m/z 305.00 [M+H]⁺.

### Step-b: Synthesis of 4-methyl-3-(1-propionyl-5-(p-tolyl)-4,5-dihydro-1H-pyrazol-3-yl)-1,5-naphthyridin-2(1H)-one

The titled compound was synthesized using the same procedure which was followed for 6-chloro-3-(5-(4-methoxyphenyl)-1-propionyl-4,5-dihydro-1H-pyrazol-3-yl)-4-methylquinolin-2(1H)-one (Example-7) using (E)-4-methyl-3-(3-(p-tolyl)acryloyl)-1,5-naphthyridin-2(1H)-one as starting material. Yield: 34%; ¹H-NMR (400 MHz, DMSO-d6): ⊐ 12.04 (bs, 1H), 8.57 (t, J=2.8, Hz, 1H), 7.70 (d, J=7.2, Hz, 1H), 7.58 (dd, J=4.8, Hz & J=8.0, Hz, 1H), 7.16-7.14 (m, 4H), 5.50 (dd, J=4.4, Hz & J=12.0, Hz, 1H), 3.82 (dd, J=12.0 Hz & J=18.0 Hz, 1H), 3.15 (dd, J=4.8 Hz & J=18.4 Hz, 1H), 2.62 (q, J=7.2 Hz, 2H), 2.59 (s, 3H), 2.28 (s, 3H), 1.03 (t, J=7.6, Hz, 3H), LCMS: m/z 375.20 [M+H]⁺.

### Example-70: Synthesis of 2,4-dimethyl-5-(1-propionyl-5-(p-tolyl)-4,5-dihydro-1H-pyrazol-3-yl)thieno[2,3-b]pyridin-6(7H)-one

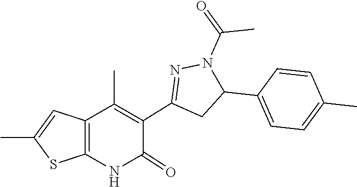

### Step-a: Synthesis of tert-butyl (3-cyano-5-methylthiophen-2-yl)carbamate

To a stirred solution of 2-amino-5-methylthiophene-3-carbonitrile (2.0 g, 14.47 mmol) in DMF (20 mL) at room temperature were added diisopropylethylamine (7.8 mL, 43.41 mmol), dimetylaminopyridine (0.17 g, 1.477 mmol) and di-tert-butyl dicarbonate (4.6 mL, 21.70 mmol). The reaction mixture was heated at 90° C. for 2 h and progress of the reaction was monitored by TLC. Reaction mixture was slowly brought to room temperature diluted with water (100 mL), extracted with ethyl acetate (2×100 mL). The combined organic layer was washed with brine (100 mL), dried over Na₂SO₄ and evaporated under reduced pressure to get the crude compound which was purified by flash column chromatography with 10-20% ethyl acetate in hexane as eluent to afford the titled compound as a yellow solid. Yield: 2.3 g (66%); ¹H-NMR (400 MHz, DMSO-d6): ⊐ 10.92 (bs, 1H), 7.09 (d, J=1.2 Hz, 1H), 2.45 (s, 3H), 1.41 (s, 9H).

### Step-b: Synthesis of tert-butyl (3-acetyl-5-methylthiophen-2-yl)carbamate

To a stirred solution of tert-butyl (3-cyano-5-methylthiophen-2-yl)carbamate (2.3 g, 9.62 mmol) in dry THF (25 mL) at 0° C. was added methyl magnesium bromide (32.8 mL, 96.0 mmol, 3 M in diethyl ether) slowly in dropwise manner for about 10-15 min. The reaction mixture was heated at 50° C. for 2 to 3 h and progress of the reaction was monitored by TLC. Reaction mixture was slowly brought to room temperature and cooled to 0° C., quenched with 2N HCl (100 mL) and then extracted with ethyl acetate (2×150 mL). Combined organic layer was washed with water (100 mL), brine (50 mL), dried over Na₂SO₄ and concentrated under reduced pressure to get the crude compound which was purified by column chromatography and eluted with 20-25% ethyl acetate in hexane to afford the titled compound as a solid. Yield: 1.03 g (41%); ¹H-NMR (400 MHz, DMSO-d6): [symbol] 10.92 (bs, 1H), 6.76 (d, J=1.2 Hz, 1H), 2.42 (s, 3H), 2.36 (s, 3H), 1.52 (s, 9H).

### Step-c: Synthesis of 1-(2-amino-5-methylthiophen-3-yl)ethan-1-one

To a stirred solution of tert-butyl (3-acetyl-5-methylthiophen-2-yl)carbamate (1 g, 3.90 mmol) in dry DCM (10 mL) at 0° C. was added 4 m HCl The reaction mixture was stirred for 5 to 6 h at room temperature. Progress of the reaction was monitored by TLC. Reaction mixture was concentrated under reduced pressure quenched and then diluted with ethyl acetate (150 mL). Organic layer was washed with saturate NaHCO₃ (50 mL) water (50 mL), brine (50 mL), dried over Na₂SO₄ and concentrated under reduced pressure to get the crude compound which was purified by column chromatography and eluted with 30-35% ethyl acetate in hexane to afford the titled compound as a solid. Yield: 0.48 g (79%); ¹H-NMR (400 MHz, DMSO-d6): [symbol] 7.89 (bs, 2H), 6.63 (d, J=1.2 Hz, 1H), 2.21 (s, 3H), 2.19 (s, 3H).

### Step-d: Synthesis of N-(3-acetyl-5-methylthiophen-2-yl)-3-oxobutanamide

To a stirred solution of 1-(2-amino-5-methylthiophen-3-yl)ethan-1-one (0.485 g 3.10 mmol) in xylene (10 mL) was added 2,2,6-trimethyl-4H-1,3-dioxin-4-one (0.6 mL, 4.66 mmol) and the reaction mixture was heated at 140° C. for 3 h. Progress of the reaction was monitored by TLC. The reaction mixture was cooled to room temperature and diluted with water (20 mL) and extracted with ethyl acetate (2×50 mL). The combined organic layer was washed with brine (50 mL), dried over Na₂SO₄, filtered and evaporated under reduced pressure to get the crude compound which was purified by flash column chromatography using 20-30% EtOAc in hexane as eluent to afford the titled compound as a colourless liquid. Yield: 0.54 g (72%); ¹H-NMR (400 MHz, DMSO-d6): [symbol] 11.93 (bs, 2H), 7.08 (d, J=1.2 Hz, 1H), 3.88 (s, 3H), 2.43 (s, 3H), 2.35 (s, 3H), 2.21 (s, 3H).

### Step-e: Synthesis of 5-acetyl-2, 4-dimethylthieno[2,3-b]pyridin-6(7H)-one

A mixture of N-(3-acetyl-5-methylthiophen-2-yl)-3-oxobutanamide (0.54 g, 2.25 mmol) and cerium chloride heptahydrate (0.25 g, 0.66 mmol) was heated at 160° C. for 30 mins. Progress of the reaction was monitored by TLC. The reaction mixture was cooled to room temperature and diluted with ethyl acetate (100 mL), water (50 mL) and extracted. The organic layer was washed with brine (50 mL), dried over sodium sulfate, filtered and evaporated under reduced pressure. The residue was diluted with DCM (50 mL) and solid obtained was filtered and dried under vacuum to afford the titled compound as off-white solid. Yield: 0.16 g (33%); ¹H-NMR (400 MHz, DMSO-d6): [symbol] ⊐ 12.23 (bs, 1H), 7.03 (s, 1H), 2.46 (s, 3H), 2.35 (s, 3H), LCMS: m/z 222.00 [M+H]⁺.

### Step-f: Synthesis of (E)-2, 4-dimethyl-5-(3-(p-tolyl)acryloyl)thieno[2,3-b]pyridin-6(7H)-one

The titled compound was synthesized using the same procedure which was followed for (E)-6-Chloro-3-(3-(4-methoxyphenyl)acryloyl)-4-methylquinolin-2(1H)-one (compound-1c) using 5-acetyl-2,4-dimethylthieno[2,3-b]pyridin-6(7H)-one and 4-methylbenzaldehyde as starting materials. Yield: 89%; ¹H-NMR (400 MHz, DMSO-d6): ⊐ 7.61 (d, J=8.4, 2H), 7.37 (d, J=16.0 Hz, 1H), 7.23 (d, J=8.0 Hz, 2H), 7.07 (s, 1H), 7.04 (d J=5.2, Hz, 1H), 2.56 (s, 3H), 2.32 (s, 3H), 2.29 (s, 3H), [NH proton is not visible in NMR]; LCMS: m/z 324.1 [M+H]⁺.

### Step-g: Synthesis of 2,4-dimethyl-5-(1-propionyl-5-(p-tolyl)-4,5-dihydro-1H-pyrazol-3-yl)thieno[2,3-b]pyridin-6(7H)-one

The titled compound was synthesized using the same procedure which was followed for 6-chloro-3-(5-(4-methoxyphenyl)-1-propionyl-4,5-dihydro-1H-pyrazol-3-yl)-4-methylquinolin-2(1H)-one (Example-7) using (E)-2,4-dimethyl-5-(3-(p-tolyl)acryloyl)thieno[2,3-b]pyridin-6(7H)-one as starting material. Yield: 18%; ¹H-NMR (400 MHz, DMSO-d6): [symbol] 27.10-7.13 (m, 4H), 7.04-7.10 (s, 1H), 5.45 (dd, J=4.0 Hz & J=12.0 Hz, 1H), 3.76 (dd, J=12.0 Hz & J=18.4 Hz, 1H), 3.11 (dd, J=4.0 Hz & J=18.0 Hz, 1H), 2.60 (q, J=7.6 Hz, 2H), 2.58 (s, 3H), 2.49 (s, 3H), 2.28 (s, 3H), 1.01 (t, J=7.2, Hz, 3H), [NH proton is not visible in NMR]; LCMS: m/z 394.10 [M+H]⁺.

### Example-71: Synthesis of 5-(5-benzyl-1-propionyl-4,5-dihydro-1H-pyrazol-3-yl)-4-methylthieno[2,3-b]pyridin-6(7H)-one

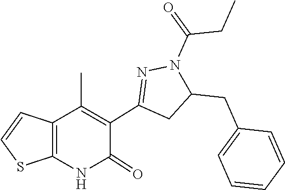 [SMILES: CCC(=O)N1N=C(c2c(C)c3ccsc3[nH]c2=O)CC1Cc1ccccc1]

### Step-a: Synthesis of (E)-4-methyl-5-(4-phenylbut-2-enoyl)thieno[2,3-b]pyridin-6(7H)-one

To a stirred solution of 5-acetyl-4-methylthieno[2,3-b]pyridin-6(7H)-one (100 mg, 0.483 mmol) and 2-phenylacetaldehyde (86 mg, 0.74 mmol), in methanol (3 mL) was added piperidine (123 mg, 1.44 mmol) and reaction mixture was heated at 90° C. in microwave 1 h. Progress of the reaction was monitored by TLC. The reaction mixture diluted with water (10 mL), obtained solid was filtered, dried under vacuum to afford the titled compound as a green solid. Yield: 110 mg (73%); LCMS: m/z 310.0 [M+H]⁺.

### Step-b: Synthesis of 5-(5-benzyl-1-propionyl-4,5-dihydro-1H-pyrazol-3-yl)-4-methylthieno[2,3-b]pyridin-6(7H)-one

The titled compound was synthesized using the same procedure which was followed for 6-chloro-3-(5-(4-methoxyphenyl)-1-propionyl-4,5-dihydro-1H-pyrazol-3-yl)-4-methylquinolin-2(1H)-one (Example-7) using (E)-4-methyl-5-(4-phenylbut-2-enoyl)thieno[2,3-b]pyridin-6(7H)-one as starting material. Yield: 14%; ¹H-NMR (400 MHz, DMSO-d6): ⊐ 7.30-7.24 (m, 5H), 7.22-7.18 (m, 2H), 4.77 (dd, J=4.4, Hz & J=9.2, Hz, 1H), 3.51 (dd, J=11.2 Hz & J=18.4 Hz, 1H), 2.97 (d, J=6.4 Hz, 1H), 2.94 (dd, J=11.2 Hz & J=18.4 Hz, 1H), 2.65 (q, J=7.6 Hz, 2H), 2.06 (s, 3H), 2.29 (s, 3H), 1.12 (t, J=7.6, Hz, 3H), [NH proton is not visible in NMR]; LCMS: m/z 379.90 [M+H]⁺.

### Example-72: Synthesis of 2-chloro-4-methyl-5-(1-propionyl-5-(p-tolyl)-4,5-dihydro-1H-pyrazol-3-yl)thieno[2,3-b]pyridin-6(7H)-one

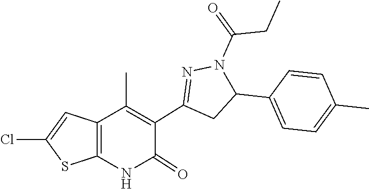 [SMILES: CCC(=O)N1N=C(c2c(C)c3cc(Cl)sc3[nH]c2=O)CC1c1ccc(C)cc1]

### Step-a: Synthesis of 2-amino-5-chlorothiophene-3-carbonitrile (72a)

To a stirred solution of 2-aminothiophene-3-carbonitrile (2.0 g, 16.10 mmol) in DMF (20 mL) at 0° C. was added N-chlorosuccinimide (2.36 g 17.71 mmol). The reaction mixture was stirred at room temperature for 16 h, and progress of the reaction was monitored by TLC. Reaction mixture was diluted with water (100 mL), extracted with ethyl acetate (2×100 mL). The combined organic layer was washed with brine (100 mL), dried over Na₂SO₄ and evaporated under reduced pressure to get the crude compound which was purified by flash column chromatography with 10-20% ethyl acetate in hexane as eluent to afford the titled compound as a yellow solid. Yield: 1.2 g, 48%; ¹H-NMR (400 MHz, DMSO-d6): ⊐ 6.57 (s, 1H), 4.78 (bs, 2H).

### Step-b: Synthesis of tert-butyl (5-chloro-3-cyanothiophen-2-yl)carbamate (72b)

The titled compound was synthesized using the same procedure which was followed for tert-butyl (3-cyano-5-methylthiophen-2-yl)carbamate (70a) using 2-amino-5-chlorothiophene-3-carbonitrile as starting material. Yield: 34%; ¹H-NMR (400 MHz, CDCl3): [symbol] 7.68 (bs, 1H), 6.75 (s, 1H), 1.54 (s, 9H).

### Step-c: Synthesis of tert-butyl (3-acetyl-5-chlorothiophen-2-yl)carbamate (72c)

The titled compound was synthesized using the same procedure which was followed for tert-butyl (3-acetyl-5-methylthiophen-2-yl)carbamate (70b) using tert-butyl (3-cyano-5-methylthiophen-2-yl)carbamate as starting material. Yield: 66%; ¹H-NMR (400 MHz, CDCl3): [symbol] 10.99 (bs, 1H), 6.96 (s, 1H), 2.42 (s, 3H), 1.54 (s, 9H).

### Step-d: Synthesis of 1-(2-amino-5-chlorothiophen-3-yl)ethan-1-one (72d)

The titled compound was synthesized using the same procedure which was followed for 1-(2-amino-5-methylthiophen-3-yl)ethan-1-one (70c) using tert-butyl (3-acetyl-5-chlorothiophen-2-yl)carbamate as starting material. Yield: 65%; ¹H-NMR (400 MHz, DMSO-d6): [symbol] 8.15 (bs, 2H), 6.96 (s, 1H), 2.24 (s, 3H).

### Step-e: Synthesis of N-(3-acetyl-5-chlorothiophen-2-yl)-3-oxobutanamide (72e)

The titled compound was synthesized using the same procedure which was followed for N-(3-acetyl-5-methylthiophen-2-yl)-3-oxobutanamide (70d) using 1-(2-amino-5-methylthiophen-3-yl)ethan-1-one as starting material. Yield: 70%; ¹H-NMR (400 MHz, DMSO-d6): [symbol] 12.14 (bs, 1H), 7.52 (s, 1H), 3.94 (s, 2H). 2.47 (s, 3H). 3.20 (s, 3H).

### Step-f: Synthesis of 5-acetyl-2-chloro-4-methylthieno[2,3-b]pyridin-6(7H)-one (72f)

The titled compound was synthesized using the same procedure which was followed for N-(3-acetyl-5-methylthiophen-2-yl)-3-oxobutanamide (70d) using N-(3-acetyl-5-methylthiophen-2-yl)-3-oxobutanamide as starting material. Yield: 41%; LCMS: m/z 242.0.1 [M+H]⁺.

### Step-g: Synthesis of (E)-2-chloro-4-methyl-5-(3-(p-tolyl)acryloyl)thieno[2,3-b]pyridin-6(7H)-one (72g)

The titled compound was synthesized using the same procedure which was followed for (E)-6-Chloro-3-(3-(4-methoxyphenyl)acryloyl)-4-methylquinolin-2(1H)-one (compound-1c) using 5-acetyl-2-chloro-4-methylthieno[2,3-b]pyridin-6(7H)-one and 4-methylbenzaldehyde as starting materials. Yield: 70%; LCMS: m/z 344.0 [M+H]⁺.

### Step-h: Synthesis of 2-chloro-4-methyl-5-(1-propionyl-5-(p-tolyl)-4,5-dihydro-1H-pyrazol-3-yl)thieno[2,3-b]pyridin-6(7H)-one

The titled compound was synthesized using the same procedure which was followed for 6-chloro-3-(5-(4-methoxyphenyl)-1-propionyl-4,5-dihydro-1H-pyrazol-3-yl)-4-methylquinolin-2(1H)-one (Example-7) using (E)-2-chloro-4-methyl-5-(3-(p-tolyl)acryloyl)thieno[2,3-b]pyridin-6(7H)-one as starting material. Yield: 15%; ¹H-NMR (400 MHz, DMSO-d6): ⊐ 7.52 (s, 1H), 7.10-7.13 (m, 4H), 5.45 (dd, J=4.0 Hz & J=11.6 Hz, 1H), 3.78 (dd, J=12.0 Hz & J=18.4 Hz, 1H), 3.10 (dd, J=4.4 Hz & J=18.4 Hz, 1H), 2.64 (q, J=7.6 Hz, 2H), 2.49 (s, 3H), 2.27 (s, 3H), 1.03 (t, J=7.2, Hz, 3H), [NH proton is not visible in NMR]; LCMS: m/z 414.00 [M+H]⁺.

### Example-73: Synthesis of 4-methyl-3-(1-propionyl-5-(p-tolyl)-4,5-dihydro-1H-pyrazol-3-yl)-1,8-naphthyridin-2(1H)-one

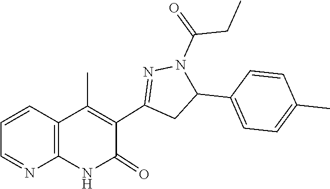 [SMILES: CCC(=O)N1N=C(c2c(C)c3cccnc3[nH]c2=O)CC1c1ccc(C)cc1]

### Step-a: Synthesis of (E)-4-methyl-3-(3-(p-tolyl)acryloyl)-1,8-naphthyridin-2(1H)-one

The titled compound was synthesized using the same procedure which was followed for (E)-6-Chloro-3-(3-(4-methoxyphenyl)acryloyl)-4-methylquinolin-2(1H)-one (compound-1c) using 3-acetyl-4-methyl-1,8-naphthyridin-2(1H)-one and 4-methylbenzaldehyde as starting materials. Yield: 38%; LCMS: m/z 305.40 [M+H]⁺.

### Step-b: Synthesis of 4-methyl-3-(1-propionyl-5-(p-tolyl)-4,5-dihydro-1H-pyrazol-3-yl)-1,8-naphthyridin-2(1H)-one

The titled compound was synthesized using the same procedure which was followed for 6-chloro-3-(5-(4-methoxyphenyl)-1-propionyl-4,5-dihydro-1H-pyrazol-3-yl)-4-methylquinolin-2(1H)-one (Example-7) using (E)-4-methyl-3-(3-(p-tolyl)acryloyl)-1,8-naphthyridin-2(1H)-one as starting material. Yield: 40%; ¹H-NMR (400 MHz, DMSO-d6): ⊐ 12.34 (bs, 1H) 8.57 (dd, J=1.6, Hz & J=4.8, Hz, 1H), 8.31 (d, J=8.4, Hz, 1H), 7.34 (dd, J=4.8, Hz & J=8.0, Hz, 1H), 7.22-7.14 (m, 4H), 5.50 (dd, J=4.4, Hz & J=11.6, Hz, 1H), 3.82 (dd, J=12.0 Hz & J=18.4 Hz, 1H), 3.10 (dd, J=4.4 Hz & J=18.4 Hz, 1H), 2.62 (q, J=7.6 Hz, 2H), 2.59 (s, 3H), 2.28 (s, 3H), 1.03 (t, J=7.2, Hz, 3H), LCMS: m/z 375.20 [M+H]⁺.

### Example-74: Synthesis of 4-methyl-3-(1-propionyl-5-(p-tolyl)-4,5-dihydro-1H-pyrazol-3-yl)-1,7-naphthyridin-2(1H)-one

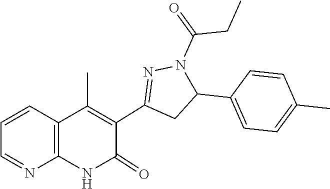 [SMILES: CCC(=O)N1N=C(c2c(C)c3cccnc3[nH]c2=O)CC1c1ccc(C)cc1]

### Step-a: Synthesis of 3-acetyl-4-methyl-1,7-naphthyridin-2(1H)-one

To a stirred solution of 1-(3-aminopyridin-4-yl)ethan-1-one (0.3 g 2.20 mmol) in xylene (10 mL) were added 2,2,6-trimethyl-4H-1,3-dioxin-4-one (0.6 mL, 4.66 mmol) and cerium chloride·heptahydrate (0.24 g, 0.66 mmol). The reaction mixture was heated at 140° C. for 3 h. Progress of the reaction was monitored by TLC. The reaction mixture was cooled to room temperature and diluted with water (50 mL) and extracted with ethyl acetate (2×100 mL). The combined organic layer was washed with brine (50 mL), dried over Na₂SO₄, filtered and evaporated under reduced pressure to get the crude compound which was purified by flash column chromatography using 2 to 3% Methanol in dichlormethane as eluent to afford the titled compound as a yellow solid. Yield: 0.21, 48%; ¹H-NMR (400 MHz, DMSO-d6): ⊐ 12.27 (bs, 1H), 8.68 (s, 1H) 8.40 (d, J=5.6 Hz, 1H), 7.76 (d, J=1.4 Hz, 1H), 2.46 (s, 3H), 2.28 (s, 3H).

### Step-b: Synthesis of (E)-4-methyl-3-(3-(p-tolyl)acryloyl)-1,7-naphthyridin-2(1H)-one

The titled compound was synthesized using the same procedure which was followed for (E)-6-Chloro-3-(3-(4-methoxyphenyl)acryloyl)-4-methylquinolin-2(1H)-one (compound-1c) using 3-acetyl-4-methyl-1,7-naphthyridin-2(1H)-one and 4-methylbenzaldehyde as starting materials. Yield: 73%; LCMS: m/z 305.40 [M+H]⁺.

### Step-c: Synthesis of 4-methyl-3-(1-propionyl-5-(p-tolyl)-4,5-dihydro-1H-pyrazol-3-yl)-1,7-naphthyridin-2(1H)-one

The titled compound was synthesized using the same procedure which was followed for 6-chloro-3-(5-(4-methoxyphenyl)-1-propionyl-4,5-dihydro-1H-pyrazol-3-yl)-4-methylquinolin-2(1H)-one (Example-7) using (E)-4-methyl-3-(3-(p-tolyl)acryloyl)-1,7-naphthyridin-2(1H)-one as starting material. Yield: 36%; ¹H-NMR (400 MHz, DMSO-d6): ⊐ 12.21 (bs, 1H), 8.69 (s, 1H), 8.41 (d, J=5.6, Hz, 1H), 7.78 (d, J=8.8, Hz, 1H), 7.19-7.14 (m, 4H), 5.50 (dd, J=4.8, Hz & J=12.80, Hz, 1H), 3.82 (dd, J=12.0 Hz & J=18.4 Hz, 1H), 3.10 (dd, J=4.4 Hz & J=18.4 Hz, 1H), 2.62 (q, J=7.6 Hz, 2H), 2.59 (s, 3H), 2.28 (s, 3H), 1.03 (t, J=7.2, Hz, 3H), LCMS: m/z 375.50 [M+H]⁺.

### Chiral Purification of Compound 74.

Step-e: 74 (A): HPLC using a chiral pack IA 5 uM, 4.6*250 mm, IPA (100) eluent at flow rate of 0.7 mL/min at 20° C. The isolated enantiomer at 12.51 min.

Step-f: 74 (B): HPLC using a chiral pack IA 5 uM, 4.6*250 mm, IPA/DCM (90/10) eluent at flow rate of 0.7 mL/min at 20° C. The isolated enantiomer at 16.94 min.

### Example-75: Synthesis of 4,7-dimethyl-3-(1-propionyl-5-(p-tolyl)-4,5-dihydro-1H-pyrazol-3-yl)quinolin-2(1H)-one

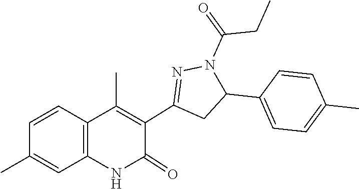

### Step-a: Synthesis of 3-acetyl-4-methyl-1,7-naphthyridin-2(1H)-one

The titled compound was synthesized using the same procedure which was followed for 3-acetyl-4-methyl-1,7-naphthyridin-2(1H)-one (compound-74a) using 1-(2-amino-4-methylphenyl)ethan-1-one as starting materials. Yield: 68%; LCMS: m/z 215.30 [M+H]⁺.

### Step-b: Synthesis of (E)-4,7-dimethyl-3-(3-(p-tolyl)acryloyl)quinolin-2(1H)-one

The titled compound was synthesized using the same procedure which was followed for (E)-6-Chloro-3-(3-(4-methoxyphenyl)acryloyl)-4-methylquinolin-2(1H)-one (compound-1c) using 3-acetyl-4-methyl-1,7-naphthyridin-2(1H)-one and 4-methylbenzaldehyde as starting materials. Yield: 59%; LCMS: m/z 317.20 [M+H]⁺.

### Step-c: Synthesis of 4,7-dimethyl-3-(1-propionyl-5-(p-tolyl)-4,5-dihydro-1H-pyrazol-3-yl)quinolin-2(1H)-one

The titled compound was synthesized using the same procedure which was followed for 6-chloro-3-(5-(4-methoxyphenyl)-1-propionyl-4,5-dihydro-1H-pyrazol-3-yl)-4-methylquinolin-2(1H)-one (Example-7) using (E)-4,7-dimethyl-3-(3-(p-tolyl)acryloyl)quinolin-2(1H)-one as starting material. Yield: 35%; ¹H-NMR (400 MHz, DMSO-d6): [symbol] 11.84 (bs, 1H), 7.75 (d, J=8.4, Hz, 1H), 7.19-7.14 (m, 5H), 7.10 (d, J=8.8, Hz, 1H), 5.48 (dd, J=4.4, Hz & J=11.6, Hz, 1H), 3.82 (dd, J=12.0 Hz & J=18.4 Hz, 1H), 3.10 (dd, J=4.4 Hz & J=18.4 Hz, 1H), 2.62 (q, J=7.6 Hz, 2H), 2.39 (s, 3H), 2.28 (s, 3H), 1.03 (t, J=7.2, Hz, 3H), LCMS: m/z 387.70 [M+H]⁺.

### Example-76: Synthesis of 4,5-dimethyl-3-(1-propionyl-5-(p-tolyl)-4,5-dihydro-1H-pyrazol-3-yl)quinolin-2(1H)-one

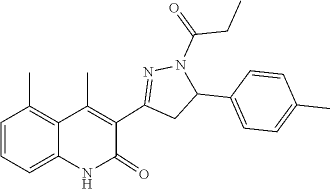 [SMILES: CCC(=O)N1N=C(c2c(C)c3c(C)cccc3[nH]c2=O)CC1c1ccc(C)cc1]

### Step-a: Synthesis of 3-acetyl-4-methyl-1,7-naphthyridin-2(1H)-one

The titled compound was synthesized using the same procedure which was followed for 3-acetyl-4-methyl-1,7-naphthyridin-2(1H)-one (compound-74a) using 1-(2-amino-6-methylphenyl)ethan-1-one as starting materials. Yield: 91%; LCMS: m/z 216.00 [M+H]⁺.

### Step-b: Synthesis of (E)-4,5-dimethyl-3-(3-(p-tolyl)acryloyl)quinolin-2(1H)-one

The titled compound was synthesized using the same procedure which was followed for (E)-6-Chloro-3-(3-(4-methoxyphenyl)acryloyl)-4-methylquinolin-2(1H)-one (compound-1c) using 3-acetyl-4-methyl-1,7-naphthyridin-2(1H)-one and 4-methylbenzaldehyde as starting materials. Yield: 51%; ¹H-NMR (400 MHz, DMSO-d6): [symbol] 11.88 (bs, 1H), 7.64 (d, J=7.6, 2H), 7.44 (d, J=11.6 Hz, 1H), 7.39 (d, J=16.8 Hz, 1H), 7.23 (d, J=7.6 Hz, 2H), 7.04 (d, J=7.2, Hz, 1H), 6.95 (d, J=16.4, Hz, 1H), 2.74 (s, 3H), 2.49 (s, 3H), 2.33 (s, 3H), LCMS: m/z 324.1 [M+H]⁺. LCMS: m/z 318.10 [M+H]⁺.

### Step-c: Synthesis of 4,5-dimethyl-3-(1-propionyl-5-(p-tolyl)-4,5-dihydro-1H-pyrazol-3-yl)quinolin-2(1H)-one

The titled compound was synthesized using the same procedure which was followed for 6-chloro-3-(5-(4-methoxyphenyl)-1-propionyl-4,5-dihydro-1H-pyrazol-3-yl)-4-methylquinolin-2(1H)-one (Example-7) using (E)-4,5-dimethyl-3-(3-(p-tolyl)acryloyl)quinolin-2(1H)-one as starting material. Yield: 22%; ¹H-NMR (400 MHz, DMSO-d6): [symbol] 11.87 (bs, 1H), 7.40 (t, J=7.6, Hz, 1H), 7.23-7.14 (m, 5H), 7.10 (d, J=7.2, Hz, 1H), 5.50 (dd, J=4.4, Hz & J=11.6, Hz, 1H), 3.76 (dd, J=12.0 Hz & J=18.4 Hz, 1H), 3.04 (dd, J=7.7 Hz & J=18.4 Hz, 1H), 2.75 (s, 3H), 2.62 (q, J=7.6 Hz, 2H), 2.64 (s, 3H), 2.28 (s, 3H), 1.03 (t, J=7.2, Hz, 3H), LCMS: m/z 388.20 [M+H]⁺.

### Example-77: Synthesis of 4-methyl-7-morpholino-3-(1-propionyl-5-(p-tolyl)-4,5-dihydro-1H-pyrazol-3-yl)quinolin-2(1H)-one

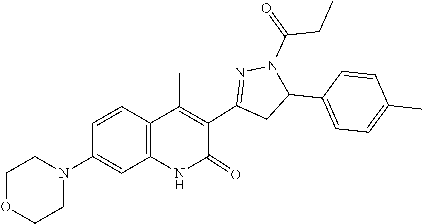 [SMILES: CCC(=O)N1N=C(c2c(C)c3ccc(N4CCOCC4)cc3[nH]c2=O)CC1c1ccc(C)cc1]

### Step-a: Synthesis of 4-morpholino-2-nitrobenzonitrile (77a)

To a stirred solution of 4-chloro-2-nitrobenzonitrile (4.0 g, 21.9 mmol) in THF (40 mL) at room temperature was added morpholine (5.66 mL, 65.70 mmol). The reaction mixture was stirred at 80° C. for 16 h, and progress of the reaction was monitored by TLC. Reaction mixture was diluted with water (100 mL), extracted with ethyl acetate (2×100 mL). The combined organic layer was washed with brine (100 mL), dried over Na₂SO₄ and evaporated under reduced pressure to get the crude compound which was purified by flash column chromatography with 10-20% ethyl acetate in hexane as eluent to afford the titled compound as an orange solid. Yield: 4.0 g, 78%; ¹H-NMR (400 MHz, DMSO-d6): [symbol] 7.88 (d, J=8.8, 1H), 7.73 (d, J=2.8 Hz, 1H), 7.37 (dd, J=2.8 Hz & J=9.2 Hz, 1H), 3.72 (t, J=4.8 Hz, 4H), 3.43 (t, J=5.2 Hz, 4H).

### Step-b: Synthesis of 2-amino-4-morpholinobenzonitrile (77b)

To a stirred solution of 4-morpholino-2-nitrobenzonitrile (2.0 g, 8.58 mmol) in methanol (20 mL) at room temperature was added 10% Pd/c (200 mg). The reaction mixture was stirred at under hydrogen balloon for 3 h, and progress of the reaction was monitored by TLC. Reaction mixture was filtered through celite, filtrate was evaporated under reduced pressure to afford the titled compound as an orange solid. Yield: 1.65 g, 94%; LCMS: m/z 204.10 [M+H]⁺.

### Step-c: Synthesis of 1-(2-amino-4-morpholinophenyl)ethan-1-one (77c)

The titled compound was synthesized using the same procedure which was followed for tert-butyl (3-acetyl-5-methylthiophen-2-yl)carbamate (70b) using tert-butyl (3-cyano-5-methylthiophen-2-yl)carbamate as starting material. Yield: 23%; LCMS: m/z 221.20 [M+H]⁺.

### Step-d: Synthesis of 3-acetyl-4-methyl-7-morpholinoquinolin-2(1H)-one (77d)

The titled compound was synthesized using the same procedure which was followed for 3-acetyl-4-methyl-1,7-naphthyridin-2(1H)-one (compound-74a) using 1-(2-amino-4-morpholinophenyl)ethan-1-one as starting materials. Yield: 15%; LCMS: m/z 287.50 [M+H]⁺.

### Step-e: Synthesis of (E)-4-methyl-7-morpholino-3-(3-(p-tolyl)acryloyl)quinolin-2(1H)-one

The titled compound was synthesized using the same procedure which was followed for (E)-6-Chloro-3-(3-(4-methoxyphenyl)acryloyl)-4-methylquinolin-2(1H)-one (compound-1c) using 3-acetyl-4-methyl-7-morpholinoquinolin-2(1H)-one and 4-methylbenzaldehyde as starting materials. Yield: 58%; ¹H-NMR (400 MHz, DMSO-d6): [symbol] 11.59 (bs, 1H), 7.66 (d, J=9.2, Hz, 1H), 7.61 (d, J=8.4, Hz, 2H), 7.41 (d, J=16.4 Hz, 1H), 7.24 (d, J=8.4 Hz, 2H), 7.04 (d, J=16.4, Hz, 1H), 6.99 (d, J=2.0, Hz, 1H), 6.72 (d, J=2.0, Hz, 1H), 3.77 (t, J=4.4, Hz, 4H), 3.23 (t, J=4.8, Hz, 4H), 2.32 (s, 3H), 2.27 (s, 3H), LCMS: m/z 389.20 [M+H]⁺.

### Step-f: Synthesis of 4-methyl-7-morpholino-3-(1-propionyl-5-(p-tolyl)-4,5-dihydro-1H-pyrazol-3-yl)quinolin-2(1H)-one

The titled compound was synthesized using the same procedure which was followed for 6-chloro-3-(5-(4-methoxyphenyl)-1-propionyl-4,5-dihydro-1H-pyrazol-3-yl)-4-methylquinolin-2(1H)-one (Example-7) using (E)-4-methyl-7-morpholino-3-(3-(p-tolyl)acryloyl)quinolin-2(1H)-one as starting material. Yield: 25%; ¹H-NMR (400 MHz, DMSO-d6): [symbol] 11.58 (bs, 1H), 7.66 (d, J=9.2, Hz, 1H), 7.23-7.14 (m, 4H), 6.97 (dd, J=2.0, Hz & J=9.2, Hz, 1H), 6.69 (d, J=2.4, Hz, 1H), 5.45 (dd, J=4.4, Hz & J=11.6, Hz, 1H), 3.80-3.72 (m, 5H), 3.34-3.21 (m, 4H), 3.11 (dd, J=4.4 Hz & J=18.0 Hz, 1H), 2.62 (q, J=7.6 Hz, 2H), 2.48 (s, 3H), 2.26 (s, 3H), 1.03 (t, J=7.2, Hz, 3H), LCMS: m/z 459.20 [M+H]⁺.

### Example-78: Synthesis of 4,8-dimethyl-3-(1-propionyl-5-(p-tolyl)-4,5-dihydro-1H-pyrazol-3-yl)-1,7-naphthyridin-2(1H)-one

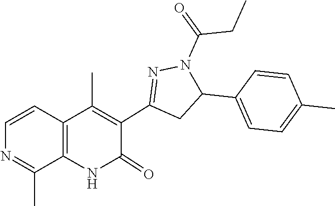 [SMILES: CCC(=O)N1N=C(c2c(C)c3ccnc(C)c3[nH]c2=O)CC1c1ccc(C)cc1]

### Step-a: Synthesis of 2-methyl-3-nitropyridin-4-ol (78a)

2-methylpyridin-4-ol (2.0 g, 18.32 mmol) was added to a mixture of fuming nitric acid and con H₂SO₄. The reaction mixture was stirred at 130° C. for 2 h, and progress of the reaction was monitored by TLC. Reaction mixture was brought to room temperature, and neutralized with sodium bicarbonate, obtained solid was filtered, dried under vacuum to afford the titled compound as yellow solid. Yield: 1.5 g, 53%; ¹H-NMR (400 MHz, DMSO-d6): [symbol] 7.51 (d, J=6.4, 1H), 6.03 (d, J=6.4 Hz, 1H), 2.13 (s, 3H).

### Step-b: Synthesis of 4-chloro-2-methyl-3-nitropyridine (78b)

2-methyl-3-nitropyridin-4-ol (2.0 g, 12.90 mmol) was added to a solution of POCl3 (10 mL) at room temperature. The reaction mixture was stirred at 110° C. for 2 h, and progress of the reaction was monitored by TLC. Reaction mixture was brought to room temperature, then evaporated under reduced pressure and quenched with ice water, neutralized with sodium bicarbonate, then extracted with ethyl acetate (2×100 mL). The combined organic layer was washed with brine (100 mL), dried over Na₂SO₄ and evaporated under reduced pressure to get the crude compound which was purified by flash column chromatography with 10-20% ethyl acetate in hexane as eluent to afford the titled compound as a reddish liquid. Yield: 0.8 g, 36%; 1H-NMR (400 MHz, CDCl₃): [symbol] 8.52 (d, J=5.2, 1H), 7.35 (d, J=5.2 Hz, 1H), 2.60 (s, 3H).

### Step-c: Synthesis of 1-(2-methyl-3-nitropyridin-4-yl)ethan-1-one (78c)

The titled compound was synthesized using the same procedure which was followed for 1-(4-Aminopyrimidin-5-yl)ethan-1-one (compound-14b) using 4-chloro-2-methyl-3-nitropyridine as starting material. Yield: 66%; ¹H-NMR (400 MHz, CDCl₃): ⊐ 18.79 (d, J=4.8, 1H), 7.40 (d, J=5.2 Hz, 1H), 2.69 (s, 3H), 2.60 (s, 3H).

### Step-d: Synthesis of 1-(3-amino-2-methylpyridin-4-yl)ethan-1-one (78d)

The titled compound was synthesized using the same procedure which was followed for 2-amino-4-morpholinobenzonitrile (compound-93b) using 1-(2-methyl-3-nitropyridin-4-yl)ethan-1-one as starting materials. Yield: 68%; ¹H-NMR (400 MHz, CDCl₃): ⊐ 7.88 (d, J=5.6, 1H), 7.37 (d, J=5.2 Hz, 1H), 6.25 (bs, 2H), 2.59 (s, 3H), 2.48 (s, 3H).

### Step-e: Synthesis of 3-acetyl-4,8-dimethyl-1,7-naphthyridin-2(1H)-one (78e)

The titled compound was synthesized using the same procedure which was followed for 3-acetyl-4-methyl-1,7-naphthyridin-2(1H)-one (compound-74a) using 1-(3-amino-2-methylpyridin-4-yl)ethan-1-one as starting materials. Yield: 61%; ¹H-NMR (400 MHz, CDCl₃): [symbol] 11.50 (bs, 1H), 8.27 (d, J=5.6, 1H), 7.60 (d, J=5.6 Hz, 1H), 2.67 (s, 3H), 2.45 (s, 3H). 2.32 (s, 3H). LCMS: m/z 217.00 [M+H]⁺.

### Step-f: Synthesis of (E)-4,8-dimethyl-3-(3-(p-tolyl)acryloyl)-1,7-naphthyridin-2(1H)-one

The titled compound was synthesized using the same procedure which was followed for (E)-6-Chloro-3-(3-(4-methoxyphenyl)acryloyl)-4-methylquinolin-2(1H)-one (compound-1c) using 3-acetyl-4,8-dimethyl-1,7-naphthyridin-2(1H)-one and 4-methylbenzaldehyde as starting materials. Yield: 81%; ¹H-NMR (400 MHz, DMSO-d6): [symbol] 11.45 (bs, 1H), 8.29 (d, J=5.6, Hz, 1H), 7.65-7.60 (m, 3H), 7.50 (d, J=16.8 Hz, 1H), 7.25 (d, J=8.0 Hz, 2H), 7.04 (d, J=16.4, Hz, 1H), 2.67 (s, 3H), 2.29 (s, 3H), 2.24 (s, 3H), LCMS: m/z 319.1 [M+H]⁺.

### Step-g: Synthesis of 4,8-dimethyl-3-(1-propionyl-5-(p-tolyl)-4,5-dihydro-1H-pyrazol-3-yl)-1,7-naphthyridin-2(1H)-one

The titled compound was synthesized using the same procedure which was followed for 6-chloro-3-(5-(4-methoxyphenyl)-1-propionyl-4,5-dihydro-1H-pyrazol-3-yl)-4-methylquinolin-2(1H)-one (Example-7) using (E)-4,8-dimethyl-3-(3-(p-tolyl)acryloyl)-1,7-naphthyridin-2(1H)-one as starting material. Yield: 32%; ¹H-NMR (400 MHz, DMSO-d6): [symbol] 11.44 (bs, 1H), 8.29 (d, J=5.6, Hz, 1H), 7.63 (d, J=5.2 Hz, 1H), 7.20-7.14 (m, 4H), 5.50 (dd, J=4.4, Hz & J=12.0, Hz, 1H), 3.86 (dd, J=12.0 Hz & J=18.4 Hz, 1H), 3.04 (dd, J=4.4 Hz & J=18.0 Hz, 1H), 2.68 (s, 3H), 2.62 (q, J=7.6 Hz, 2H), 2.59 (s, 3H), 2.28 (s, 3H), 1.03 (t, J=7.2, Hz, 3H), LCMS: m/z 389.00 [M+H]⁺.

### Chiral Purification of Compound 78.

Step-e: 78 (A): HPLC using a chiral pack IA 5 uM, 4.6*250 mm, IPA (100) eluent at flow rate of 0.7 mL/min at 20° C. The isolated enantiomer at 16.67 min, (the dextro (+) isomer).

Step-f: 78 (B): HPLC using a chiral pack IA 5 uM, 4.6*250 mm, IPA/DCM (90/10) eluent at flow rate of 0.7 mL/min at 20° C. The isolated enantiomer at 20.64 min, (the levo (−) isomer).

### Example-79: Synthesis of 6-chloro-4-methyl-3-(1-propionyl-5-(p-tolyl)-4,5-dihydro-1H-pyrazol-3-yl)-1,7-naphthyridin-2(1H)-one

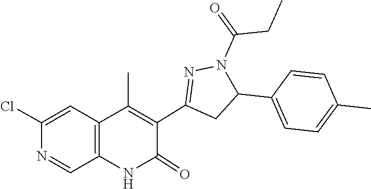 [SMILES: CCC(=O)N1N=C(c2c(C)c3cc(Cl)ncc3[nH]c2=O)CC1c1ccc(C)cc1]

### Step-a: Synthesis of 3-acetyl-6-chloro-4-methyl-1,7-naphthyridin-2(1H)-one

The titled compound was synthesized using the same procedure which was followed for 3-acetyl-4-methyl-1,7-naphthyridin-2(1H)-one (compound-74a) using 1-(5-amino-2-chloropyridin-4-yl)ethan-1-one as starting materials. Yield: 48%; LCMS: m/z 237.00 [M+H]⁺.

### Step-b: Synthesis of (E)-6-chloro-4-methyl-3-(3-(p-tolyl)acryloyl)-1,7-naphthyridin-2(1H)-one

The titled compound was synthesized using the same procedure which was followed for (E)-6-Chloro-3-(3-(4-methoxyphenyl)acryloyl)-4-methylquinolin-2(1H)-one (compound-1c) using 3-acetyl-6-chloro-4-methyl-1,7-naphthyridin-2(1H)-one and 4-methylbenzaldehyde as starting materials. Yield: 75%; LCMS: m/z 339.00 [M+H]⁺.

### Step-c: Synthesis of 6-chloro-4-methyl-3-(1-propionyl-5-(p-tolyl)-4,5-dihydro-1H-pyrazol-3-yl)-1,7-naphthyridin-2(1H)-one

The titled compound was synthesized using the same procedure which was followed for 6-chloro-3-(5-(4-methoxyphenyl)-1-propionyl-4,5-dihydro-1H-pyrazol-3-yl)-4-methylquinolin-2(1H)-one (Example-7) using (E)-6-chloro-4-methyl-3-(3-(p-tolyl)acryloyl)-1,7-naphthyridin-2(1H)-one as starting material. Yield: 20%; ¹H-NMR (400 MHz, DMSO-d6): [symbol] 12.30 (bs, 1H), 8.48 (s, 1H), 7.89 (s, 1H), 7.18-7.12 (m, 4H), 5.49 (dd, J=4.4, Hz & J=11.6, Hz, 1H), 3.77 (dd, J=12.0 Hz & J=18.4 Hz, 1H), 3.07 (dd, J=7.8 Hz & J=18.4 Hz, 1H), 2.62 (q, J=7.6 Hz, 2H), 2.53 (s, 3H), 2.28 (s, 3H), 1.03 (t, J=7.2, Hz, 3H), LCMS: m/z 409.10 [M+H]⁺.

### Example-80: Synthesis of 7-chloro-4-methyl-3-(1-propionyl-5-(p-tolyl)-4,5-dihydro-1H-pyrazol-3-yl)quinolin-2(1H)-one

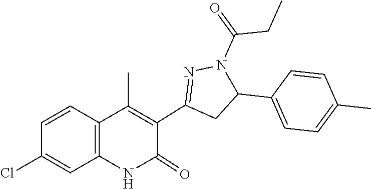 [SMILES: CCC(=O)N1N=C(c2c(C)c3ccc(Cl)cc3[nH]c2=O)CC1c1ccc(C)cc1]

### Step-a: Synthesis of 3-acetyl-7-chloro-4-methylquinolin-2(1H)-one

The titled compound was synthesized using the same procedure which was followed for 3-acetyl-4-methyl-1,7-naphthyridin-2(1H)-one (compound-74a) using 1-(2-amino-4-chlorophenyl)ethan-1-one as starting materials. Yield: 55%; ¹H-NMR (400 MHz, DMSO-d6): ⊐ 12.08 (bs, 1H), 8.68 (s, 1H) 7.86 (d, J=8.8 Hz, 1H), 7.35 (s, 1H), 7.30 (d, J=8.4 Hz, 1H), 2.45 (s, 3H), 2.32 (s, 3H). LCMS: m/z 235.90 [M+H]⁺.

### Step-b: Synthesis of (E)-7-chloro-4-methyl-3-(3-(p-tolyl)acryloyl)quinolin-2(1H)-one

The titled compound was synthesized using the same procedure which was followed for (E)-6-Chloro-3-(3-(4-methoxyphenyl)acryloyl)-4-methylquinolin-2(1H)-one (compound-1c) using 3-acetyl-7-chloro-4-methylquinolin-2(1H)-one and 4-methylbenzaldehyde as starting materials. Yield: 93%; LCMS: m/z 337.90 [M+H]⁺.

### Step-c: Synthesis of 7-chloro-4-methyl-3-(1-propionyl-5-(p-tolyl)-4,5-dihydro-1H-pyrazol-3-yl)quinolin-2(1H)-one

The titled compound was synthesized using the same procedure which was followed for 6-chloro-3-(5-(4-methoxyphenyl)-1-propionyl-4,5-dihydro-1H-pyrazol-3-yl)-4-methylquinolin-2(1H)-one (Example-7) using (E)-7-chloro-4-methyl-3-(3-(p-tolyl)acryloyl)quinolin-2(1H)-one as starting material. Yield: 62%; ¹H-NMR (400 MHz, DMSO-d6): ⊐ 12.02 (bs, 1H), 7.88 (d, J=8.8, Hz, 1H), 7.73 (d, J=2.0, Hz, 1H), 7.30 (dd, J=2.0, Hz & J=8.8, Hz, 1H), 7.19-7.11 (m, 4H), 5.47 (dd, J=4.4, Hz & J=11.6, Hz, 1H), 3.77 (dd, J=12.0 Hz & J=18.4 Hz, 1H), 3.07 (dd, J=4.4 Hz & J=18.0 Hz, 1H), 2.62 (q, J=7.6 Hz, 2H), 2.53 (s, 3H), 2.28 (s, 3H), 1.03 (t, J=7.2, Hz, 3H), LCMS: m/z 408.10 [M+H]⁺.

### Chiral Purification of Compound 80.

Step-e: 80 (A): HPLC using a chiral pack IA 5 uM, 4.6*250 mm, IPA (100) eluent at flow rate of 0.7 mL/min at 20° C. The isolated enantiomer at 17.01 min.

Step-f: 80 (B): HPLC using a chiral pack IA 5 uM, 4.6*250 mm, IPA/DCM (90/10) eluent at flow rate of 0.7 mL/min at 20° C. The isolated enantiomer at 25.77 min.

### Example-81: Synthesis of 4,6-dimethyl-3-(1-propionyl-5-(p-tolyl)-4,5-dihydro-1H-pyrazol-3-yl)-1,7-naphthyridin-2(1H)-one

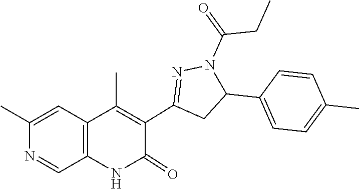

### Step-a: Synthesis of 3-acetyl-4,6-dimethyl-1,7-naphthyridin-2(1H)-one

The titled compound was synthesized using the same procedure which was followed for 3-acetyl-4-methyl-1,7-naphthyridin-2(1H)-one (compound-74a) using 1-(5-amino-2-methylpyridin-4-yl)ethan-1-one as starting materials. Yield: 58%; ¹H-NMR (400 MHz, DMSO-d6): [symbol] 12.14 (bs, 1H), 8.56 (s, 1H) 7.61 (s, 1H), 2.53 (s, 3H), 2.45 (s, 3H) 2.30 (s, 3H). LCMS: m/z 217.30 [M+H]⁺.

### Step-b: Synthesis of (E)-4,6-dimethyl-3-(3-(p-tolyl)acryloyl)-1,7-naphthyridin-2(1H)-one

The titled compound was synthesized using the same procedure which was followed for (E)-6-Chloro-3-(3-(4-methoxyphenyl)acryloyl)-4-methylquinolin-2(1H)-one (compound-1c) using 3-acetyl-4,6-dimethyl-1,7-naphthyridin-2(1H)-one and 4-methylbenzaldehyde as starting materials. Yield: 89%; LCMS: m/z 318.80 [M+H]⁺.

### Step-c: Synthesis of 4,6-dimethyl-3-(1-propionyl-5-(p-tolyl)-4,5-dihydro-1H-pyrazol-3-yl)-1,7-naphthyridin-2(1H)-one

The titled compound was synthesized using the same procedure which was followed for 6-chloro-3-(5-(4-methoxyphenyl)-1-propionyl-4,5-dihydro-1H-pyrazol-3-yl)-4-methylquinolin-2(1H)-one (Example-7) using (E)-4,6-dimethyl-3-(3-(p-tolyl)acryloyl)-1,7-naphthyridin-2(1H)-one as starting material. Yield: 26%; ¹H-NMR (400 MHz, DMSO-d6): [symbol] 12.07 (bs, 1H), 8.58 (s, 1H) 7.63 (s, 1H), 7.19-7.13 (m, 4H), 5.50 (dd, J=4.8, Hz & J=12.0, Hz, 1H), 3.81 (dd, J=12.0 Hz & J=18.4 Hz, 1H), 3.11 (dd, J=4.8 Hz & J=18.0 Hz, 1H), 2.62 (q, J=7.6 Hz, 2H), 2.59 (s, 3H), 2.53 (s, 3H), 2.28 (s, 3H), 1.03 (t, J=7.2, Hz, 3H), LCMS: m/z 388.90 [M+H]⁺.

### Example-82: Synthesis of 4-methyl-7-(4-methylpiperazin-1-yl)-3-(1-propionyl-5-(p-tolyl)-4,5-dihydro-1H-pyrazol-3-yl)quinolin-2(1H)-one

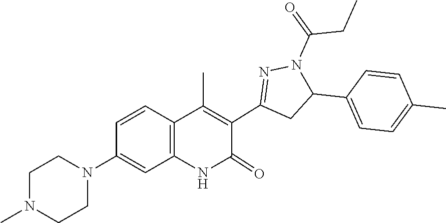

### Step-a: Synthesis of 3-acetyl-4-methyl-7-(4-methylpiperazin-1-yl)quinolin-2(1H)-one

The titled compound was synthesized using the same procedure which was followed for 3-acetyl-4-methyl-1,7-naphthyridin-2(1H)-one (compound-74a) using 1-(2-amino-4-(4-methylpiperazin-1-yl)phenyl)ethan-1-one as starting materials. Yield: 39%; ¹H-NMR (400 MHz, DMSO-d6): [symbol] 12.57 (bs, 1H), 7.60 (d, J=9.2 Hz, 1H), 6.92 (d, J=2.8 Hz, 1H), 6.74 (d, J=2.4 Hz, 1H), 345-3.40 (m, 4H), 2.45 (s, 3H), 2.35-2.30 (m, 4H), 2.27 (s, 3H). LCMS: m/z 300.30 [M+H]⁺.

### Step-b: Synthesis of (E)-4-methyl-7-(4-methylpiperazin-1-yl)-3-(3-(p-tolyl)acryloyl)quinolin-2(1H)-one

The titled compound was synthesized using the same procedure which was followed for (E)-6-Chloro-3-(3-(4-methoxyphenyl)acryloyl)-4-methylquinolin-2(1H)-one (compound-1c) using 3-acetyl-4-methyl-7-(4-methylpiperazin-1-yl)quinolin-2(1H)-one and 4-methylbenzaldehyde as starting materials. Yield: 89%; LCMS: m/z 402.40 [M+H]⁺.

### Step-c: Synthesis of 4-methyl-7-(4-methylpiperazin-1-yl)-3-(1-propionyl-5-(p-tolyl)-4,5-dihydro-1H-pyrazol-3-yl)quinolin-2(1H)-one

The titled compound was synthesized using the same procedure which was followed for 6-chloro-3-(5-(4-methoxyphenyl)-1-propionyl-4,5-dihydro-1H-pyrazol-3-yl)-4-methylquinolin-2(1H)-one (Example-7) using (E)-4-methyl-7-(4-methylpiperazin-1-yl)-3-(3-(p-tolyl)acryloyl)quinolin-2(1H)-one as starting material. Yield: 48%; ¹H-NMR (400 MHz, DMSO-d6): ⊐ 11.51 (bs, 1H), 7.66 (d, J=9.2, Hz, 1H), 7.19-7.12 (m, 4H), 6.96 (dd, J=2.0, Hz & J=9.6, Hz, 1H), 6.91 (d, J=2.0, Hz, 1H), 5.45 (dd, J=4.4, Hz & J=11.6, Hz, 1H), 3.79 (dd, J=12.0 Hz & J=18.4 Hz, 1H), 3.45 (s, 3H), 3.28-3.27 (m, 4H), 3.11 (dd, J=4.4 Hz & J=18.0 Hz, 1H), 2.62 (q, J=7.6 Hz, 2H), 2.58-2.50 (m, 4H), 2.27 (s, 3H), 2.23 (s, 3H), 1.03 (t, J=7.2, Hz, 3H), LCMS: m/z 472.50 [M+H]⁺.

### Chiral Purification of Compound 82.

Step-e: 82 (A): HPLC using a chiral pack IA 5 uM, 4.6*250 mm, IPA (100) eluent at flow rate of 0.7 mL/min at 20° C. The isolated enantiomer at 31.40 min.

Step-f: 82 (B): HPLC using a chiral pack IA 5 uM, 4.6*250 mm, IPA/DCM (90/10) eluent at flow rate of 0.7 mL/min at 20° C. The isolated enantiomer at 44.02 min.

### Example-83: Synthesis of 7-fluoro-4-methyl-3-(1-propionyl-5-(p-tolyl)-4,5-dihydro-1H-pyrazol-3-yl)quinolin-2(1H)-one

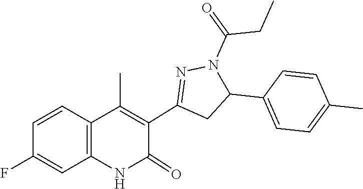 [SMILES: CCC(=O)N1N=C(c2c(C)c3ccc(F)cc3[nH]c2=O)CC1c1ccc(C)cc1]

### Step-a: Synthesis of 3-acetyl-7-fluoro-4-methylquinolin-2(1H)-one

The titled compound was synthesized using the same procedure which was followed for 3-acetyl-4-methyl-1,7-naphthyridin-2(1H)-one (compound-74a) using 1-(2-amino-4-fluorophenyl)ethan-1-one as starting materials. Yield: 39%; ¹H-NMR (400 MHz, DMSO-d6): [symbol] 712.06 (bs, 1H), 7.90 (t, J=8.0 Hz, 1H), 7.15 (dd, J=8.8, Hz & J=18.8, Hz, 2H), 2.45 (s, 3H), 2.33 (s, 3H). LCMS: m/z 220.20 [M+H]⁺.

### Step-b: Synthesis of (E)-7-fluoro-4-methyl-3-(3-(p-tolyl)acryloyl)quinolin-2(1H)-one

The titled compound was synthesized using the same procedure which was followed for (E)-6-Chloro-3-(3-(4-methoxyphenyl)acryloyl)-4-methylquinolin-2(1H)-one (compound-1c) using 3-acetyl-7-fluoro-4-methylquinolin-2(1H)-one and 4-methylbenzaldehyde as starting materials. Yield: 89%; LCMS: m/z 321.90 [M+H]⁺.

### Step-c: Synthesis of 7-fluoro-4-methyl-3-(1-propionyl-5-(p-tolyl)-4,5-dihydro-1H-pyrazol-3-yl)quinolin-2(1H)-one

The titled compound was synthesized using the same procedure which was followed for 6-chloro-3-(5-(4-methoxyphenyl)-1-propionyl-4,5-dihydro-1H-pyrazol-3-yl)-4-methylquinolin-2(1H)-one (Example-7) using (E)-7-fluoro-4-methyl-3-(3-(p-tolyl)acryloyl)quinolin-2(1H)-one as starting material. Yield: 48%; ¹H-NMR (400 MHz, DMSO-d6): ⊐ 12.01 (bs, 1H), 7.93 (t, J=6.4 Hz, 1H), 7.19-7.08 (m, 6H), 5.50 (dd, J=4.4, Hz & J=12.4, Hz, 1H), 3.81 (dd, J=12.0 Hz & J=18.4 Hz, 1H), 3.11 (dd, J=4.8 Hz & J=18.0 Hz, 1H), 2.62 (q, J=7.6 Hz, 2H), 2.59 (s, 3H), 2.28 (s, 3H), 1.03 (t, J=7.2, Hz, 3H), LCMS: m/z 391.90 [M+H]⁺.

### Example-84: Synthesis of 4,7-dimethyl-3-(1-propionyl-5-(p-tolyl)-4,5-dihydro-1H-pyrazol-3-yl)-1,8-naphthyridin-2(1H)-one

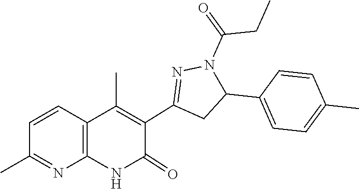

### Step-a: Synthesis of 3-acetyl-4,7-dimethyl-1,8-naphthyridin-2(1H)-one

The titled compound was synthesized using the same procedure which was followed for 3-acetyl-4-methyl-1,7-naphthyridin-2(1H)-one (compound-74a) using 1-(2-amino-6-methylpyridin-3-yl)ethan-1-one as starting materials. Yield: 56%; 1H-NMR (400 MHz, DMSO-d6): [symbol] 9.01 (bs, 1H), 8.68 (s, 1H) 7.96 (d, J=8.4 Hz, 1H), 7.11 (d, J=8.0 Hz, 1H), 2.61 (s, 3H), 2.60 (s, 3H), 2.42 (s, 3H). LCMS: m/z 216.80 [M+H]⁺.

### Step-b: Synthesis of (E)-4,7-dimethyl-3-(3-(p-tolyl)acryloyl)-1,8-naphthyridin-2(1H)-one

The titled compound was synthesized using the same procedure which was followed for (E)-6-Chloro-3-(3-(4-methoxyphenyl)acryloyl)-4-methylquinolin-2(1H)-one (compound-1c) using 3-acetyl-4,7-dimethyl-1,8-naphthyridin-2(1H)-one and 4-methylbenzaldehyde as starting materials. Yield: 78%; LCMS: m/z 318.80 [M+H]⁺.

### Step-c: Synthesis of 4,7-dimethyl-3-(1-propionyl-5-(p-tolyl)-4,5-dihydro-1H-pyrazol-3-yl)-1,8-naphthyridin-2(1H)-one

The titled compound was synthesized using the same procedure which was followed for 6-chloro-3-(5-(4-methoxyphenyl)-1-propionyl-4,5-dihydro-1H-pyrazol-3-yl)-4-methylquinolin-2(1H)-one (Example-7) using (E)-4,7-dimethyl-3-(3-(p-tolyl)acryloyl)-1,8-naphthyridin-2(1H)-one as starting material. Yield: 28%; 1H-NMR (400 MHz, DMSO-d6): ⊐ 12.20 (bs, 1H), 8.18 (d, J=8.4, Hz, 1H), 7.21-7.13 (m, 5H), 5.48 (dd, J=4.4, Hz & J=12.0, Hz, 1H), 3.81 (dd, J=12.0 Hz & J=18.4 Hz, 1H), 3.10 (dd, J=4.4 Hz & J=18.4 Hz, 1H), 2.62 (q, J=7.6 Hz, 2H), 2.54 (s, 3H), 2.49 (s, 3H), 2.27 (s, 3H), 1.03 (t, J=7.2, Hz, 3H), LCMS: m/z 389.00 [M+H]⁺.

### Example-85: Synthesis of 4-methyl-3-(1-propionyl-5-(p-tolyl)-4,5-dihydro-1H-pyrazol-3-yl)-1,6-naphthyridin-2(1H)-one

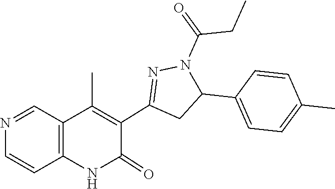 [SMILES: CCC(=O)N1N=C(c2c(C)c3cnccc3[nH]c2=O)CC1c1ccc(C)cc1]

### Step-a: Synthesis of 3-acetyl-4-methyl-1,6-naphthyridin-2(1H)-one

The titled compound was synthesized using the same procedure which was followed for 3-acetyl-4-methyl-1,7-naphthyridin-2(1H)-one (compound-74a) using 1-(4-aminopyridin-3-yl)ethan-1-one as starting materials. Yield: 42%; ¹H-NMR (400 MHz, DMSO-d6): ⊐ 12.25 (bs, 1H), 9.02 (s, 1H), 8.53 (d, J=4.0 Hz, 1H), 7.23 (d, J=6.0 Hz, 1H), 2.45 (s, 3H), 2.40 (s, 3H). LCMS: m/z 202.80[M+H]⁺.

### Step-b: Synthesis of (E)-4-methyl-3-(3-(p-tolyl)acryloyl)-1,6-naphthyridin-2(1H)-one

The titled compound was synthesized using the same procedure which was followed for (E)-6-Chloro-3-(3-(4-methoxyphenyl)acryloyl)-4-methylquinolin-2(1H)-one (compound-1c) using 3-acetyl-4-methyl-1,6-naphthyridin-2(1H)-one and 4-methylbenzaldehyde as starting materials. Yield: 93%; LCMS: m/z 304.90 [M+H]⁺.

### Step-c: Synthesis of 4-methyl-3-(1-propionyl-5-(p-tolyl)-4,5-dihydro-1H-pyrazol-3-yl)-1,6-naphthyridin-2(1H)-one

The titled compound was synthesized using the same procedure which was followed for 6-chloro-3-(5-(4-methoxyphenyl)-1-propionyl-4,5-dihydro-1H-pyrazol-3-yl)-4-methylquinolin-2(1H)-one (Example-7) using (E)-4-methyl-3-(3-(p-tolyl)acryloyl)-1,6-naphthyridin-2(1H)-one as starting material. Yield: 15%; ¹H-NMR (400 MHz, DMSO-d6): ⊐ 12.20 (bs, 1H), 9.05 (s, 1H), 8.53 (d, J=4.0, Hz, 1H), 7.24 (d, J=5.6, Hz, 1H), 7.18-7.14 (m, 4H), 5.50 (dd, J=4.4, Hz & J=11.6, Hz, 1H), 3.77 (dd, J=12.0 Hz & J=18.4 Hz, 1H), 3.09 (dd, J=4.4 Hz & J=18.0 Hz, 1H), 2.62 (q, J=7.6 Hz, 2H), 2.59 (s, 3H), 2.28 (s, 3H), 1.03 (t, J=7.2, Hz, 3H), LCMS: m/z 375.00 [M+H]⁺.

### Example-86: Synthesis of 4-methyl-7-morpholino-3-(1-propionyl-5-(p-tolyl)-4,5-dihydro-1H-pyrazol-3-yl)-1,8-naphthyridin-2(1H)-one

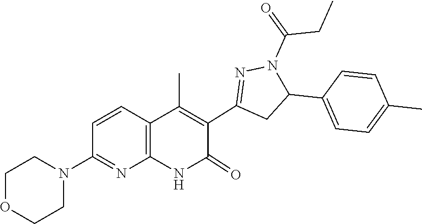 [SMILES: CCC(=O)N1N=C(c2c(C)c3ccc(N4CCOCC4)nc3[nH]c2=O)CC1c1ccc(C)cc1]

### Step-a: Synthesis of 6-chloro-2-((4-methoxybenzyl)amino)nicotinic acid

To a stirred solution of 2,6-dichloronicotinic acid (10 g, 52.08 mmol) in dry DMF (40 mL) was added K₂CO₃ (14.38 g, 104.16 mmol) and then cooled the reaction mixture to 0° C. Finally PMB-Cl (9.28 g, 67.70 mmol) was added in drop-wise manner for 10 min and the reaction mixture was heated at 100° C. for 16 h. Reaction progress was monitored by TLC. Reaction mixture was poured onto ice-water (50 mL), and acidified with 10% KHSO₄, obtained solid was filtered, dried under vacuum to afford the titled compound as an off white solid. Yield: 13 g (86%). LCMS: m/z 293.00 [M+H]⁺.

### Step-b: Synthesis of 6-chloro-N-methoxy-2-((4-methoxybenzyl)amino)-N-methylnicotinamide

To a stirred solution of 6-chloro-2-((4-methoxybenzyl)amino)nicotinic acid (3.0 g, 10.27 mmol) in DMF (30 mL), N,O-dimethylhydroxylamine hydrochloride (1.49 g, 15.40 mmol), DIPEA (8.0 mL, 46.21 mmol) and then HATU (5.86 g, 15.40 mmol) were added at 0° C. The reaction mixture was stirred at room temperature for 5 h, and progress of the reaction was monitored by TLC. Reaction mixture was diluted with water (100 mL), extracted with ethyl acetate (2×100 mL). The combined organic layer was washed with brine (100 mL), dried over Na₂SO₄ and evaporated under reduced pressure to get the crude compound which was purified by flash column chromatography with 10-20% ethyl acetate in hexane as eluent to afford the titled compound as an orange solid. Yield: 2.5 g, 73%; LCMS: m/z 336.0[M+H]⁺.

### Step-c: Synthesis of 1-(6-chloro-2-((4-methoxybenzyl)amino)pyridin-3-yl)ethan-1-one

To a stirred solution of 6-chloro-N-methoxy-2-((4-methoxybenzyl)amino)-N-methylnicotinamide (2.5 g, 7.462 mmol) in dry THF (25 mL) at 0° C. was added methyl magnesium bromide (8.7 mL, 26.11 mmol, 3 M in diethyl ether) slowly in dropwise manner for about 10-15 min. The reaction mixture was stirred at room temperature for 2 to 3 h, and progress of the reaction was monitored by TLC. Reaction mixture was cooled to 0° C., quenched with saturated NH₄Cl (100 mL) and then extracted with ethyl acetate (2×150 mL). Combined organic layer was washed with water (100 mL), brine (50 mL), dried over Na₂SO₄ and concentrated under reduced pressure to get the crude compound which was purified by column chromatography and eluted with 10-15% ethyl acetate in hexane to afford the titled compound as a solid. Yield: 1.5 g (71%); LCMS: m/z 290.90[M+H]⁺.

### Step-d: Synthesis of 1-(2-((4-methoxybenzyl)amino)-6-morpholinopyridin-3-yl)ethan-1-one

To a stirred solution of 1-(6-chloro-2-((4-methoxybenzyl)amino)pyridin-3-yl)ethan-1-one (1.5 g, 6.52 mmol) in DMSO (15 mL) at room temperature was added morpholine (0.9 mL, 9.78 mmol) and K₂CO₃ (2.7 g, 19.56 mmol). The reaction mixture was stirred at 120° C. for 2 h, and progress of the reaction was monitored by TLC. Reaction mixture was diluted with water (100 mL), extracted with ethyl acetate (2×100 mL). The combined organic layer was washed with brine (100 mL), dried over Na₂SO₄ and evaporated under reduced pressure to get the crude compound which was purified by flash column chromatography with 15-20% ethyl acetate in hexane as eluent to afford the titled compound as a yellow solid. Yield: 1.13 g, 64%; LCMS: m/z 341.90[M+H]⁺.

### Step-e: Synthesis of 1-(2-amino-6-morpholinopyridin-3-yl)ethan-1-one

To a stirred solution of 1-(2-((4-methoxybenzyl)amino)-6-morpholinopyridin-3-yl)ethan-1-one (500 mg, 1.46 mmol) in DCM (10 mL), was added TFA (3.0 mL) at 0° C. The reaction mixture was stirred at room temperature for 16 h, and progress of the reaction was monitored by TLC. Reaction mixture was evaporated under reduced pressure diluted with ethyl acetate (100 mL). The organic layer was washed with saturated NaHCO₃ (50 mL) and brine (50 mL), dried over Na₂SO₄ and evaporated under reduced pressure to get the crude compound which was purified by flash column chromatography with 30-40% ethyl acetate in hexane as eluent to afford the titled compound as an brown solid. Yield: 220 mg, 68%; ¹H-NMR (400 MHz, DMSO-d6): ⊐ 7.85 (d, J=8.8 Hz, 1H), 6.11 (d, J=9.2 Hz, 1H), 3.64 (t, J=4.4 Hz, 4H), 3.57 (t, J=5.2 Hz, 4H), 2.34 (s, 3H), LCMS: m/z 341.90[M+H]⁺.

### Step-f: Synthesis of 3-acetyl-4-methyl-7-morpholino-1,8-naphthyridin-2(1H)-one

The titled compound was synthesized using the same procedure which was followed for 3-acetyl-4-methyl-1,7-naphthyridin-2(1H)-one (compound-74a) using 1-(2-amino-6-morpholinopyridin-3-yl)ethan-1-one as starting materials. Yield: 58%; LCMS: m/z 288.0 [M+H]⁺.

### Step-g: Synthesis of (E)-4-methyl-7-morpholino-3-(3-(p-tolyl)acryloyl)-1,8-naphthyridin-2(1H)-one

The titled compound was synthesized using the same procedure which was followed for (E)-6-Chloro-3-(3-(4-methoxyphenyl)acryloyl)-4-methylquinolin-2(1H)-one (compound-1c) using 3-acetyl-4-methyl-7-morpholino-1,8-naphthyridin-2(1H)-one and 4-methylbenzaldehyde as starting materials. Yield: 63%; LCMS: m/z 390.10 [M+H]⁺.

### Step-h: Synthesis of 4-methyl-7-morpholino-3-(1-propionyl-5-(p-tolyl)-4,5-dihydro-1H-pyrazol-3-yl)-1,8-naphthyridin-2(1H)-one

The titled compound was synthesized using the same procedure which was followed for 6-chloro-3-(5-(4-methoxyphenyl)-1-propionyl-4,5-dihydro-1H-pyrazol-3-yl)-4-methylquinolin-2(1H)-one (Example-7) using (E)-4-methyl-7-morpholino-3-(3-(p-tolyl)acryloyl)-1,8-naphthyridin-2(1H)-one as starting material. Yield: 62%; ¹H-NMR (400 MHz, DMSO-d6): ⊐ 12.73 (bs, 1H), 7.99 (d, J=9.2, Hz, 1H), 7.19-7.11 (m, 4H), 6.80 (d, J=9.2, Hz, 1H), 5.45 (dd, J=4.4, Hz & J=12.0, Hz, 1H), 3.80 (dd, J=12.0 Hz & J=18.4 Hz, 1H), 3.70 (t, J=4.8 Hz, 4H), 3.62 (t, J=4.8 Hz, 4H), 3.10 (dd, J=4.4 Hz & J=18.0 Hz, 1H), 2.62 (q, J=7.6 Hz, 2H), 2.45 (s, 3H), 2.27 (s, 3H), 1.03 (t, J=7.2, Hz, 3H), LCMS: m/z 460.20 [M+H]⁺.

### Example-87: Synthesis of 7-(2-methoxyethoxy)-4-methyl-3-(1-propionyl-5-(p-tolyl)-4,5-dihydro-1H-pyrazol-3-yl)-1,8-naphthyridin-2(1H)-one

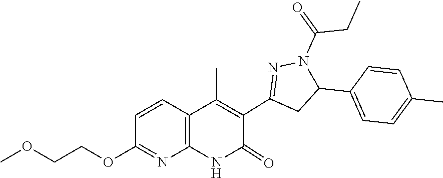

### Step-a: Synthesis of 1-(2-amino-6-(2-methoxyethoxy)pyridin-3-yl)ethan-1-one

To a stirred solution of 1-(2-amino-6-chloropyridin-3-yl)ethan-1-one (100 mg, 0.588 mmol) in dry THF (10 mL) was added KOtBu (132 mg, 1.17 mmol) and 2-methoxyethan-1-ol (67 mg, 0.88 mmol) at room temperature then reaction mixture was heated at 70° C. for 3 h. Reaction progress was monitored by TLC. Reaction mixture was diluted with water (20 mL), extracted with ethyl acetate (2×50 mL). The combined organic layer was washed with brine (50 mL), dried over Na₂SO₄ and evaporated under reduced pressure to get the crude compound which was purified by flash column chromatography with 15-20% ethyl acetate in hexane as eluent to afford the titled compound as off white solid. Yield: 30 mg, 25%;

¹H-NMR (400 MHz, DMSO-d6): [symbol] 7.87 (d, J=8.8 Hz, 1H), 6.11 (d, J=8.4 Hz, 1H), 4.47 (t, J=4.4 Hz, 2H), 3.71 (t, J=4.4 Hz, 2H), 2.47 (s, 3H). LCMS: m/z 211.0[M+H]⁺.

### Step-b: Synthesis of 3-acetyl-7-(2-methoxyethoxy)-4-methyl-1,8-naphthyridin-2(1H)-one

To a stirred solution of 1-(2-amino-6-(2-methoxyethoxy)pyridin-3-yl)ethan-1-one (65 mg 0.308 mmol) in dioxane (10 mL) was added 2,2,6-trimethyl-4H-1,3-dioxin-4-one (66 mg, 0.46 mmol). The reaction mixture was heated at 120° C. for 5 h. Progress of the reaction was monitored by TLC. The reaction mixture was cooled to room temperature and diluted with water (10 mL) and extracted with ethyl acetate (2×50 mL). The combined organic layer was washed with brine (50 mL), dried over Na₂SO₄, filtered and evaporated under reduced pressure to get the crude compound which was purified by flash column chromatography using 2 to 3% Methanol in dichloromethane as eluent to afford the titled compound as a brown solid. Yield: 40 mg, 48%; LCMS: m/z 277.0[M+H]⁺.

### Step-c: Synthesis of (E)-7-(2-methoxyethoxy)-4-methyl-3-(3-(p-tolyl)acryloyl)-1,8-naphthyridin-2(1H)-one

The titled compound was synthesized using the same procedure which was followed for (E)-6-Chloro-3-(3-(4-methoxyphenyl)acryloyl)-4-methylquinolin-2(1H)-one (compound-1c) using 3-acetyl-7-(2-methoxyethoxy)-4-methyl-1,8-naphthyridin-2(1H)-one and 4-methylbenzaldehyde as starting materials. Yield: 57%; LCMS: m/z 379.10 [M+H]⁺.

### Step-d: Synthesis of 7-(2-methoxyethoxy)-4-methyl-3-(1-propionyl-5-(p-tolyl)-4,5-dihydro-1H-pyrazol-3-yl)-1,8-naphthyridin-2(1H)-one

The titled compound was synthesized using the same procedure which was followed for 6-chloro-3-(5-(4-methoxyphenyl)-1-propionyl-4,5-dihydro-1H-pyrazol-3-yl)-4-methylquinolin-2(1H)-one (Example-7) using (E)-7-(2-methoxyethoxy)-4-methyl-3-(3-(p-tolyl)acryloyl)-1,8-naphthyridin-2(1H)-one as starting material. Yield: 38%; ¹H-NMR (400 MHz, DMSO-d6): [symbol] 12.73 (bs, 1H), 8.19 (d, J=8.8, Hz, 1H), 7.18-7.13 (m, 4H), 6.76 (d, J=4.0, Hz, 1H), 5.47 (dd, J=4.4, Hz & J=12.0, Hz, 1H), 4.47 (t, J=4.0 Hz, 2H), 3.77 (dd, J=12.0 Hz & J=18.4 Hz, 1H), 3.69 (t, J=4.4 Hz, 2H), 3.29 (s, 3H), 3.10 (dd, J=4.4 Hz & J=18.0 Hz, 1H), 2.62 (q, J=7.6 Hz, 2H), 2.52 (s, 3H), 2.27 (s, 3H), 1.03 (t, J=7.2, Hz, 3H), LCMS: m/z 449.10 [M+H]⁺.

### List of Abbreviations Used in the Present Invention are Given Below

ACN Acetonitrile
Aq Aqueous
bs broad singlet
° C. degree Celsius
CHCl₃ Chloroform
d doublet
DCM Dichloromethane
dd doublet of doublets
DIPEA N, N-Diisopropylethylamine
DMAP 4-Dimethylaminopyridine
DMF Dimethylformamide
DMSO Dimethyl sulfoxide
EtOAc Ethyl acetate
EtOH Ethanol
g gram
h hour
HCl Hydrochloric acid
Hz Hertz
HPLC High-performance liquid chromatography
LC Liquid chromatography
m multiplet
MeOH Methanol
mg milligram
MHz Megahertz
min minutes
mL milliliter
mM millimolar
mmol Millimoles
MS Mass spectrometry
MW Microwave
NaOH Sodium hydroxide
NBS N-Bromosuccinimide
nM nanomolar
NMR Nuclear magnetic resonance spectroscopy
O/N Over night
Prep. Preparative
RT Room temperature
t triplet
TEA Triethylamine
THF Tetrahydrofuran
TLC Thin-layer chromatography

### Pharmacological Activity:

### Antiproliferation Assay in MDA MB 231 Cells

MDA MB 231 cells were seeded in 96 well plate at a seeding density of 250 cells/well. The cells were allowed to settle overnight under standard incubator conditions (37° C., 5% CO₂). Next day treatment with increasing dose of test compounds and reference compounds were done to the respective wells and the cells were further incubated under standard cell culture conditions for another 4 days. On the 5^{th} day, media was replenished with fresh treatment and cells were incubated for a total of 9 day treatment period. On the day of termination, appropriate volume of Cell Titer Glo reagent was added to the treatment (and control) wells and the plates were kept on a plate shaker @300 RPM for 15 min. Luminiscence reading (RLU) was Plates were further kept in the dark for an additional 10 min before taking

RLU is directly proportional to ATP content and is directly related to number of metabolically active cells in the respective well.

### Calculation:

a. Blank Correction: RLU of the Wells Containing No Cells is Considered as Blank and Subtracted from all RLU of the Treated and Control Wells

$$\% inhibition = 100 - [ ( Treated RLU - Blank RLU ) / ( Control RLU - Blank RLU ) * 100 ]$$

TABLE 2
(Proliferation assay in MDAMB231 cell line):

| Example No | % inhibition @ 10 μM |
|---|---|
| 1 | + |
| 2 | + |
| 3 | + |
| 4 | + |
| 5 | ++ |
| 6 | + |
| 7 | + |
| 8 | + |
| 9 | ++ |
| 10 | + |
| 11 | + |
| 12 | + |
| 13 | + |
| 14 | ++ |
| 15 | + |
| 16 | + |
| 17 | + |
| 18 | ++ |
| 19 | ++ |
| 20 | + |
| 21 | + |
| 22 | + |
| 23 | + |
| 24 | + |
| 25 | ++ |
| 26 | ++ |
| 27 | ++ |
| 28 | + |
| 29 | + |
| 30 | + |
| 31 | ++ |
| 32 | ++ |
| 33 | ++ |
| 34 | ++ |
| 35 | ++ |
| 36 | ++ |
| 37 | + |
| 38 | + |
| 39 | + |
| 40 | ++ |
| 41 | + |
| 42 | + |
| 43 | + |
| 44 | + |
| 45 | + |
| 46 | + |
| 47 | ++ |
| 48 | ++ |
| 49 | ++ |
| 50 | ++ |
| 51 | ++ |
| 52 | + |
| 53 | ++ |
| 54 | + |
| 55 | + |
| 56 | + |
| 57 | + |
| 58 | ++ |
| 59 | + |
| 60 | + |
| 61 | + |
| 62 | + |
| 63 | + |
| 64 | + |
| 65 | + |
| 66 | + |
| 67 | ++ |
| 68 | + |
| 69 | + |
| 70 | + |
| 71 | + |
| 72 | + |
| 73 | + |
| 74 | + |
| 75 | + |
| 76 | + |
| 77 | + |
| 78 | + |
| 79 | + |
| 80 | + |
| 81 | ++ |
| 82 | + |
| 83 | + |
| 84 | + |
| 85 | + |
| 86 | ++ |
| 87 | + |

Table key:
≥50% +;
& <50% ++

TABLE 3
(Proliferation assay in MDAMB231 cell line):

| Example No | % inhibition @ 10 μM |
|---|---|
| 20A | + |
| 20B | + |
| 28A | + |
| 28B | + |
| 33A | ++ |
| 33B | ++ |
| 37A | + |
| 37B | + |
| 45A | ++ |
| 45B | + |
| 60A | + |
| 60B | + |
| 66A | + |
| 66B | + |
| 74A | + |
| 74B | + |
| 78A | ++ |
| 78B | + |
| 80A | ++ |
| 80B | + |
| 82A | + |
| 82B | + |

1. Novel bicyclic compounds of formula (I) 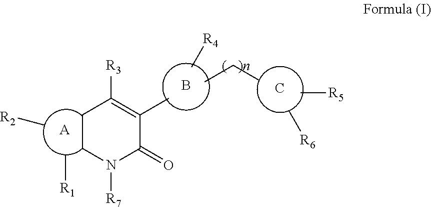
ring A is selected from 6-10 membered aryl, 5-10 membered heteroaryl, 6-10 membered cyclic ring system and 3-10 membered heterocyclyl;
ring B is selected from 6-10 membered aryl, 5-10 membered heteroaryl and 3-10 membered heterocyclyl;
ring C is selected from 6-10 membered aryl, 5-10 membered heteroaryl and 3-10 membered heterocyclyl;
wherein either of A or B or C is optionally substituted by one or more, identical or different substituents;
R¹ and R² is present or absent and when present is selected from the group consisting of hydrogen, halogen, alkyl, alkoxy, —C(O)alkyl, cycloalkyl, heterocyclyl, aryl, heteroaryl; wherein any of the group is optionally substituted by one or more, identical or different substituents;
R³ is selected from the group consisting of hydrogen, halogen, amino, nitro, alkyl, haloalkyl, alkoxy, hydroxyalkyl, cycloalkyl, heterocyclyl, heteroaryl;
R⁴ is selected from the group consisting of hydrogen, alkyl, —C(O)—R^{4a} wherein any of the group is optionally substituted by one or more, identical or different substituents;
R^{4a} is selected from the group consisting of hydrogen, alkyl, haloalkyl, cycloalkyl, heterocyclyl; wherein any of the group is optionally substituted by one or more, identical or different substituents;
R⁵ and R⁶ are same or different and each individually is selected from the group consisting of hydrogen, halogen, amino, nitro, alkyl, haloalkyl, cycloalkyl, alkoxy, acylamino, alkylamino, dialkylamino, heterocyclyl; wherein any of the group is optionally substituted by one or more, identical or different substituents;
R⁷ is selected from the group consisting of hydrogen, alkyl and
“n” is 0 to 2.

2. The compound of formula (I) according to claim 1, or a pharmaceutically acceptable salt or a stereoisomer thereof, in which the ring A along with the attached ring is specifically represented by

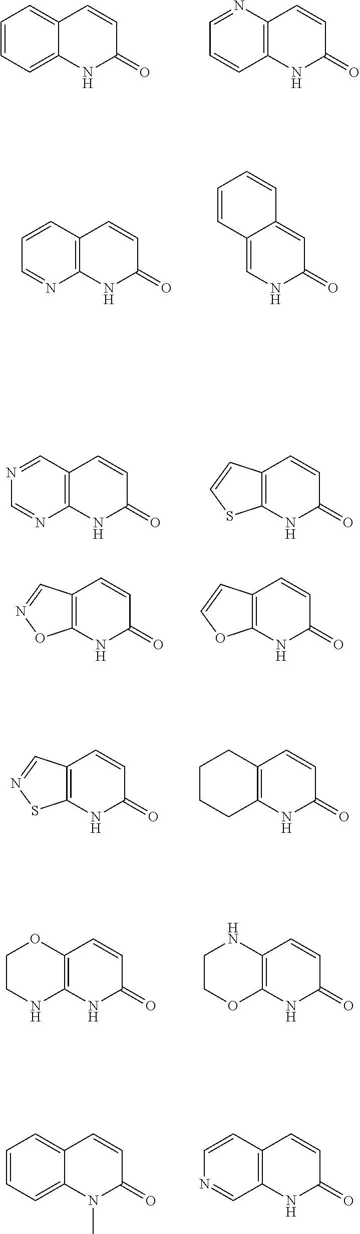

3. The novel bicyclic compounds of formula (I) according to claim 1 wherein the compound is formula (Ia) 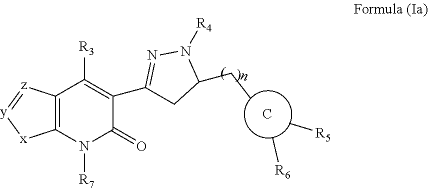
or a pharmaceutically acceptable salt or a pharmaceutically acceptable regioisomer thereof;
wherein,
“x” is selected from O, S or N R′, wherein R′ is selected from hydrogen, alkyl;
“y” and “z” independently are selected from N, CR″, wherein R″ is selected from hydrogen, halogen, alkyl;
ring C is selected from 6-10 membered aryl, 5-10 membered heteroaryl and 3-10 membered heterocyclyl, optionally substituted by one or more, identical or different substituents;
R³ is selected from the group consisting of hydrogen, halogen, amino, nitro, alkyl, haloalkyl, alkoxy, hydroxyalkyl, cycloalkyl, heterocyclyl, heteroaryl;
R⁴ is selected from the group consisting of hydrogen, alkyl, —C(O)—R^{4a} wherein any of the group is optionally substituted by one or more, identical or different substituents;
R^{4a} is selected from the group consisting of hydrogen, alkyl, haloalkyl, cycloalkyl, heterocyclyl; wherein any of the group is optionally substituted by one or more, identical or different substituents;
R⁵ and R⁶ are same or different and each individually is selected from the group consisting of hydrogen, halogen, amino, nitro, alkyl, haloalkyl, cycloalkyl, alkoxy, acylamino, alkylamino, dialkylamino, heterocyclyl; wherein any of the group is optionally substituted by one or more, identical or different substituents;
R⁷ is selected from the group consisting of hydrogen, alkyl and
“n” is 0 to 2.

4. The novel bicyclic compounds of formula (I) according to claim 1 wherein the compound is formula (Ib) 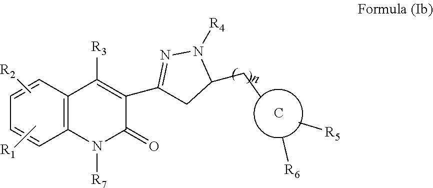
or a pharmaceutically acceptable salt or a pharmaceutically acceptable regioisomer thereof;
wherein,
ring C is selected from 6-10 membered aryl, 5-10 membered heteroaryl and 3-10 membered heterocyclyl, optionally substituted by one or more, identical or different substituents;
R¹ and R² is present or absent and when present is selected from the group consisting of hydrogen, halogen, alkyl, alkoxy, —C(O)alkyl, cycloalkyl, heterocyclyl, aryl, heteroaryl; wherein any of the group is optionally substituted by one or more, identical or different substituents;
R³ is selected from the group consisting of hydrogen, halogen, amino, nitro, alkyl, haloalkyl, alkoxy, hydroxyalkyl, cycloalkyl, heterocyclyl, heteroaryl;
R⁴ is selected from the group consisting of hydrogen, alkyl, —C(O)—R^{4a} wherein any of the group is optionally substituted by one or more, identical or different substituents;
R^{4a} is selected from the group consisting of hydrogen, alkyl, haloalkyl, cycloalkyl, heterocyclyl; wherein any of the group is optionally substituted by one or more, identical or different substituents;
R⁵ and R⁶ are same or different and each individually is selected from the group consisting of hydrogen, halogen, amino, nitro, alkyl, haloalkyl, cycloalkyl, alkoxy, acylamino, alkylamino, dialkylamino heterocyclyl; wherein any of the group is optionally substituted by one or more, identical or different substituents;
R⁷ is selected from the group consisting of hydrogen, alkyl and
“n” is 0 to 2.

5. The novel bicyclic compounds of formula (I) according to claim 1 wherein the compound is formula (Ic) 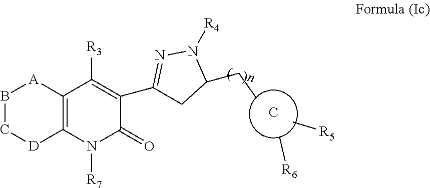
or a pharmaceutically acceptable salt or a pharmaceutically acceptable regioisomer thereof;
wherein,
ring C is selected from 6-10 membered aryl, 5-10 membered heteroaryl and 3-10 membered heterocyclyl, optionally substituted by one or more, identical or different substituents;
“A”, “B”, “C”, and “D” independently are selected from O, S, NR′, C(R″)₂, or any of the two A and B or C and D represent CR′ form a double bond, wherein R′ is absent or when present is selected from hydrogen, alkyl and R″ is hydrogen, halogen and alkyl;
R³ is selected from the group consisting of hydrogen, halogen, amino, nitro, alkyl, haloalkyl, alkoxy, hydroxyalkyl, cycloalkyl, heterocyclyl, heteroaryl;
R⁴ is selected from the group consisting of hydrogen, alkyl, —C(O)—R^{4a} wherein any of the group is optionally substituted by one or more, identical or different substituents;
R^{4a} is selected from the group consisting of hydrogen, alkyl, haloalkyl, cycloalkyl, heterocyclyl, wherein any of the group is optionally substituted by one or more, identical or different substituents;
R⁵ and R⁶ are same or different and each individually is selected from the group consisting of hydrogen, halogen, amino, nitro, alkyl, haloalkyl, cycloalkyl, alkoxy, acylamino, alkylamino, dialkylamino, heterocyclyl; wherein any of the group is optionally substituted by one or more, identical or different substituents;
R⁷ is selected from the group consisting of hydrogen, alkyl and
“n” is 0 to 2.

6. The novel bicyclic compounds of formula (I) according to claim 1 wherein the compound is formula (Id) 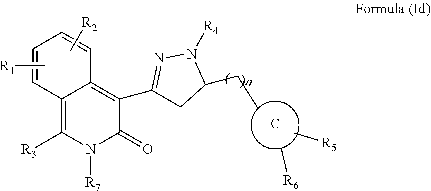
or a pharmaceutically acceptable salt or a pharmaceutically acceptable regioisomer thereof;
wherein,
ring C is selected from 6-10 membered aryl, 5-10 membered heteroaryl and 3-10 membered heterocyclyl, optionally substituted by one or more, identical or different substituents;
R¹ and R² is present or absent and when present is selected from the group consisting of hydrogen, halogen, alkyl, alkoxy, —C(O)alkyl, cycloalkyl, heterocyclyl, aryl, heteroaryl, wherein any of the group is optionally substituted by one or more, identical or different substituents;
R³ is selected from the group consisting of hydrogen, halogen, amino, nitro, alkyl, haloalkyl, hydroxyalkyl, cycloalkyl, heterocyclyl, heteroaryl;
R⁴ is selected from the group consisting of hydrogen, alkyl, —C(O)—R^{4a} wherein any of the group is optionally substituted by one or more, identical or different substituents;
R^{4a} is selected from the group consisting of hydrogen, alkyl, haloalkyl, cycloalkyl, heterocyclyl, wherein any of the group is optionally substituted by one or more, identical or different substituents;
R⁵ and R⁶ are same or different and each individually is selected from the group consisting of hydrogen, halogen, amino, nitro, alkyl, haloalkyl, cycloalkyl, alkoxy, acylamino, alkylamino, dialkylamino, heterocyclyl; wherein any of the group is optionally substituted by one or more, identical or different substituents;
R⁷ is selected from the group consisting of hydrogen, alkyl and
“n” is 0 to 2.

7. The novel bicyclic compounds of formula (I) according to claim 3, wherein the ring C is specifically represented by

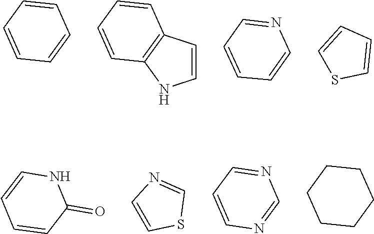

8. A novel bicyclic compound selected from the group consisting of
1. 6-Chloro-3-(5-(4-methoxyphenyl)-1-propionyl-4,5-dihydro-1H-pyrazol-3-yl)-4-methylquinolin-2(1H)-one
2. 6-Chloro-3-(1-(cyclopropanecarbonyl)-5-(4-methoxyphenyl)-4,5-dihydro-1H-pyrazol-3-yl)-4-methylquinolin-2(1H)-one
3. 6-chloro-3-(5-(4-methoxyphenyl)-1-(tetrahydro-2H-pyran-4-carbonyl)-4,5-dihydro-1H-pyrazol-3-yl)-4-methylquinolin-2(1H)-one
4. 6-chloro-3-(5-(4-methoxyphenyl)-1-(tetrahydrofuran-3-carbonyl)-4,5-dihydro-1H-pyrazol-3-yl)-4-methylquinolin-2(1H)-one
5. 6-Chloro-3-(5-(4-methoxyphenyl)-1-methyl-4,5-dihydro-1H-pyrazol-3-yl)-4-methylquinolin-2(1H)-one
6. 6-Chloro-4-methyl-3-(5-(1-methyl-1H-indol-6-yl)-1-propionyl-4,5-dihydro-1H-pyrazol-3-yl)quinolin-2(1H)-one
7. 6-chloro-3-(5-(6-methoxypyridin-3-yl)-1-propionyl-4,5-dihydro-1H-pyrazol-3-yl)-4-methylquinolin-2(1H)-one
8. N-(4-(3-(6-chloro-4-methyl-2-oxo-1,2-dihydroquinolin-3-yl)-1-propionyl-4,5-dihydro-1H-pyrazol-5-yl)phenyl)propionamide
9. 3-(5-(4-Methoxyphenyl)-1-propionyl-4,5-dihydro-1H-pyrazol-3-yl)-4-methyl-1,5-naphthyridin-2(1H)-one
10. 4-Methyl-3-(5-(1-methyl-1H-indol-6-yl)-1-propionyl-4,5-dihydro-1H-pyrazol-3-yl)quinolin-2(1H)-one
11. 3-(5-(4-Methoxyphenyl)-1-propionyl-4,5-dihydro-1H-pyrazol-3-yl)-4-methylquinolin-2(1H)-one
12. 4-Methyl-3-(5-(1-methyl-1H-indol-3-yl)-1-propionyl-4,5-dihydro-1H-pyrazol-3-yl)quinolin-2(1H)-one
13. 3-(5-(4-Methoxyphenyl)-1-propionyl-4,5-dihydro-1H-pyrazol-3-yl)-4-methyl-1,8-naphthyridin-2(1H)-one
14. 6-(5-(4-Methoxyphenyl)-1-propionyl-4,5-dihydro-1H-pyrazol-3-yl)-5-methylpyrido-[2,3-d]pyrimidin-7(8H)-one
15. 6-bromo-3-(5-(4-methoxyphenyl)-1-propionyl-4,5-dihydro-1H-pyrazol-3-yl)-4-methylquinolin-2(1H)-one
16. 6-chloro-3-(5-(4-methoxyphenyl)-1-propionyl-4,5-dihydro-1H-pyrazol-3-yl)-4-methyl-1,5-naphthyridin-2(1H)-one
17. 6-Chloro-3-(5-(4-methoxyphenyl)-1-propionyl-4,5-dihydro-1H-pyrazol-3-yl)quinolin-2(1H)-one
18. 6-chloro-3-(5-(4-methoxyphenyl)-1-propionyl-4,5-dihydro-1H-pyrazol-3-yl)-1,4-dimethylquinolin-2(1H)-one
19. 4-(5-(4-methoxyphenyl)-1-propionyl-4,5-dihydro-1H-pyrazol-3-yl)isoquinolin-3(2H)-one
20. 5-(5-(4-Methoxyphenyl)-1-propionyl-4,5-dihydro-1H-pyrazol-3-yl)-4-methylthieno-[2,3-b]pyridin-6(7H)-one
21. 5-(5-(6-methoxypyridin-3-yl)-1-propionyl-4,5-dihydro-1H-pyrazol-3-yl)-4-methylthieno[2,3-b]pyridin-6(7H)-one
22. 5-(5-(4-methoxyphenyl)-1-propionyl-4,5-dihydro-1H-pyrazol-3-yl)thieno[2,3-b]pyridin-6(7H)-one
23. 6-chloro-4-cyclopropyl-3-(5-(4-methoxyphenyl)-1-propionyl-4,5-dihydro-1H-pyrazol-3-yl)quinolin-2(1H)-one
24. 6-chloro-4-methyl-3-(5-(1-methyl-1H-indol-3-yl)-1-propionyl-4,5-dihydro-1H-pyrazol-3-yl)quinolin-2(1H)-one
25. 5-(5-(5-methoxypyridin-2-yl)-1-propionyl-4,5-dihydro-1H-pyrazol-3-yl)-4-methylthieno[2,3-b]pyridin-6(7H)-one
26. 4-methyl-5-(5-(6-oxo-1,6-dihydropyridin-3-yl)-1-propionyl-4,5-dihydro-1H-pyrazol-3-yl)thieno[2,3-b]pyridin-6(7H)-one
27. 5-(5-(4-fluorophenyl)-1-propionyl-4,5-dihydro-1H-pyrazol-3-yl)-4-methylthieno[2,3-b]pyridin-6(7H)-one
28. 5-(5-(4-chlorophenyl)-1-propionyl-4,5-dihydro-1H-pyrazol-3-yl)-4-methylthieno[2,3-b]pyridin-6(7H)-one
29. 5-(5-(5-methoxythiophen-2-yl)-1-propionyl-4,5-dihydro-1H-pyrazol-3-yl)-4-methylthieno[2,3-b]pyridin-6(7H)-one
30. 5-(5-(2-methoxythiazol-5-yl)-1-propionyl-4,5-dihydro-1H-pyrazol-3-yl)-4-methylthieno[2,3-b]pyridin-6(7H)-one
31. 5-(5-(5-bromothiophen-3-yl)-1-propionyl-4,5-dihydro-1H-pyrazol-3-yl)-4-methylthieno[2,3-b]pyridin-6(7H)-one
32. 4-methyl-5-(5-phenyl-1-propionyl-4,5-dihydro-1H-pyrazol-3-yl)thieno[2,3-b]pyridin-6(7H)-one
33. 4-methyl-5-(1-propionyl-5-(thiophen-3-yl)-4,5-dihydro-1H-pyrazol-3-yl)thieno[2,3-b]pyridin-6(7H)-one
34. 5-(5-(4-bromothiophen-2-yl)-1-propionyl-4,5-dihydro-1H-pyrazol-3-yl)-4-methylthieno[2,3-b]pyridin-6(7H)-one
35. 4-methyl-5-(1-propionyl-5-(pyridin-4-yl)-4,5-dihydro-1H-pyrazol-3-yl)thieno[2,3-b]pyridin-6(7H)-one
36. 4-methyl-5-(1-propionyl-5-(pyridin-3-yl)-4,5-dihydro-1H-pyrazol-3-yl)thieno[2,3-b]pyridin-6(7H)-one
37. 4-methyl-5-(1-propionyl-5-(p-tolyl)-4,5-dihydro-1H-pyrazol-3-yl)thieno[2,3-b]pyridin-6(7H)-one
38. 4-methyl-5-(5-(4-(4-methylpiperazin-1-yl)phenyl)-1-propionyl-4,5-dihydro-1H-pyrazol-3-yl)thieno[2,3-b]pyridin-6(7H)-one
39. 5-(5-(4-(dimethylamino)phenyl)-1-propionyl-4,5-dihydro-1H-pyrazol-3-yl)-4-methylthieno[2,3-b]pyridin-6(7H)-one
40. 3-chloro-5-(5-(4-methoxyphenyl)-1-propionyl-4,5-dihydro-1H-pyrazol-3-yl)-4-methylthieno[2,3-b]pyridin-6(7H)-one
41. 5-(5-(5-methylthiophen-2-yl)-1-propionyl-4,5-dihydro-1H-pyrazol-3-yl)-4-methylthieno[2,3-b]pyridin-6(7H)-one
42. 4-methyl-5-(1-propionyl-5-(4-(trifluoromethyl)phenyl)-4,5-dihydro-1H-pyrazol-3-yl)thieno[2,3-b]pyridin-6(7H)-one
43. 5-(5-(4-(difluoromethyl)phenyl)-1-propionyl-4,5-dihydro-1H-pyrazol-3-yl)-4-methylthieno[2,3-b]pyridin-6(7H)-one
44. 5-(5-(4-ethylphenyl)-1-propionyl-4,5-dihydro-1H-pyrazol-3-yl)-4-methylthieno[2,3-b]pyridin-6(7H)-one
45. 4-methyl-5-(5-(6-methylpyridin-3-yl)-1-propionyl-4,5-dihydro-1H-pyrazol-3-yl)thieno[2,3-b]pyridin-6(7H)-one
46. 4-methyl-5-(1-propionyl-5-(4-(pyrrolidin-1-yl)phenyl)-4,5-dihydro-1H-pyrazol-3-yl)thieno[2,3-b]pyridin-6(7H)-one
47. 5-(5-(4-cyclopropylphenyl)-1-propionyl-4,5-dihydro-1H-pyrazol-3-yl)-4-methylthieno[2,3-b]pyridin-6(7H)-one
48. 4-methyl-5-(5-(2-methylthiazol-4-yl)-1-propionyl-4,5-dihydro-1H-pyrazol-3-yl)thieno[2,3-b]pyridin-6(7H)-one
49. 5-(5-(4-isopropylphenyl)-1-propionyl-4,5-dihydro-1H-pyrazol-3-yl)-4-methylthieno[2,3-b]pyridin-6(7H)-one
50. 5-(5-(4,6-dimethylpyridin-3-yl)-1-propionyl-4,5-dihydro-1H-pyrazol-3-yl)-4-methylthieno[2,3-b]pyridin-6(7H)-one
51. 4-methyl-5-(5-(2-methylpyrimidin-5-yl)-1-propionyl-4,5-dihydro-1H-pyrazol-3-yl)thieno[2,3-b]pyridin-6(7H)-one
52. 5-(5-(5-chloro-6-methylpyridin-3-yl)-1-propionyl-4,5-dihydro-1H-pyrazol-3-yl)-4-methylthieno[2,3-b]pyridin-6(7H)-one
53. 5-(5-(4-methoxy-6-methylpyridin-3-yl)-1-propionyl-4,5-dihydro-1H-pyrazol-3-yl)-4-methylthieno[2,3-b]pyridin-6(7H)-one
54. 5-(5-(5,6-dimethylpyridin-3-yl)-1-propionyl-4,5-dihydro-1H-pyrazol-3-yl)-4-methylthieno[2,3-b]pyridin-6(7H)-one
55. 5-(5-(5-fluoro-6-methylpyridin-3-yl)-1-propionyl-4,5-dihydro-1H-pyrazol-3-yl)-4-methylthieno[2,3-b]pyridin-6(7H)-one
56. 5-(5-(4-chloro-6-methylpyridin-3-yl)-1-propionyl-4,5-dihydro-1H-pyrazol-3-yl)-4-methylthieno[2,3-b]pyridin-6(7H)-one
57. 4-methyl-5-(5-(2-methylthiazol-5-yl)-1-propionyl-4,5-dihydro-1H-pyrazol-3-yl)thieno[2,3-b]pyridin-6(7H)-one
58. 5-(5-cyclohexyl-1-propionyl-4,5-dihydro-1H-pyrazol-3-yl)-4-methylthieno[2,3-b]pyridin-6(7H)-one
59. 6-chloro-4-methyl-3-(1-propionyl-5-(p-tolyl)-4,5-dihydro-1H-pyrazol-3-yl)quinolin-2(1H)-one
60. 4-methyl-3-(1-propionyl-5-(p-tolyl)-4,5-dihydro-1H-pyrazol-3-yl)quinolin-2(1H)-one
61. 6-chloro-4-methyl-3-(1-propionyl-5-(p-tolyl)-4,5-dihydro-1H-pyrazol-3-yl)-1,5-naphthyridin-2(1H)-one
62. 5-(1-acetyl-5-(p-tolyl)-4,5-dihydro-1H-pyrazol-3-yl)-4-methyl-7,7a-dihydrothieno[2,3-b]pyridin-6(7H)-one
63. 5-(1-isobutyryl-5-(p-tolyl)-4,5-dihydro-1H-pyrazol-3-yl)-4-methyl-7,7a-dihydrothieno[2,3-b]pyridin-6(7H)-one
64. 4-methyl-5-(5-(p-tolyl)-1-(2,2,2-trifluoroacetyl)-4,5-dihydro-1H-pyrazol-3-yl)-7,7a-dihydrothieno[2,3-b]pyridin-6(7H)-one
65. 5-(5-(2-chloro-4-methylphenyl)-1-propionyl-4,5-dihydro-1H-pyrazol-3-yl)-4-methylthieno[2,3-b]pyridin-6(7H)-one
66. 5-(5-(2-fluoro-4-methylphenyl)-1-propionyl-4,5-dihydro-1H-pyrazol-3-yl)-4-methylthieno[2,3-b]pyridin-6(7H)-one
67. 6-chloro-4-methyl-3-(1-propionyl-5-(p-tolyl)-4,5-dihydro-1H-pyrazol-3-yl)-1,8-naphthyridin-2(1H)-one
68. 5-(5-(4-(fluoromethyl)phenyl)-1-propionyl-4,5-dihydro-1H-pyrazol-3-yl)-4-methylthieno[2,3-b]pyridin-6(7H)-one
69. 4-methyl-3-(1-propionyl-5-(p-tolyl)-4,5-dihydro-1H-pyrazol-3-yl)-1,5-naphthyridin-2(1H)-one
70. 2,4-dimethyl-5-(1-propionyl-5-(p-tolyl)-4,5-dihydro-1H-pyrazol-3-yl)thieno[2,3-b]pyridin-6(7H)-one
71. 5-(5-benzyl-1-propionyl-4,5-dihydro-1H-pyrazol-3-yl)-4-methylthieno[2,3-b]pyridin-6(7H)-one
72. 2-chloro-4-methyl-5-(1-propionyl-5-(p-tolyl)-4,5-dihydro-1H-pyrazol-3-yl)thieno[2,3-b]pyridin-6(7H)-one
73. 4-methyl-3-(1-propionyl-5-(p-tolyl)-4,5-dihydro-1H-pyrazol-3-yl)-1,8-naphthyridin-2(1H)-one
74. 4-methyl-3-(1-propionyl-5-(p-tolyl)-4,5-dihydro-1H-pyrazol-3-yl)-1,7-naphthyridin-2(1H)-one
75. 4,7-dimethyl-3-(1-propionyl-5-(p-tolyl)-4,5-dihydro-1H-pyrazol-3-yl)quinolin-2(1H)-one
76. 4,5-dimethyl-3-(1-propionyl-5-(p-tolyl)-4,5-dihydro-1H-pyrazol-3-yl)quinolin-2(1H)-one
77. 4-methyl-7-morpholino-3-(1-propionyl-5-(p-tolyl)-4,5-dihydro-1H-pyrazol-3-yl)quinolin-2(1H)-one
78. 4,8-dimethyl-3-(1-propionyl-5-(p-tolyl)-4,5-dihydro-1H-pyrazol-3-yl)-1,7-naphthyridin-2(1H)-one
79. 6-chloro-4-methyl-3-(1-propionyl-5-(p-tolyl)-4,5-dihydro-1H-pyrazol-3-yl)-1,7-naphthyridin-2(1H)-one′
80. 7-chloro-4-methyl-3-(1-propionyl-5-(p-tolyl)-4,5-dihydro-1H-pyrazol-3-yl)quinolin-2(1H)-one
81. 4,6-dimethyl-3-(1-propionyl-5-(p-tolyl)-4,5-dihydro-1H-pyrazol-3-yl)-1,7-naphthyridin-2(1H)-one
82. 4-methyl-7-(4-methylpiperazin-1-yl)-3-(1-propionyl-5-(p-tolyl)-4,5-dihydro-1H-pyrazol-3-yl)quinolin-2(1H)-one
83. 7-fluoro-4-methyl-3-(1-propionyl-5-(p-tolyl)-4,5-dihydro-1H-pyrazol-3-yl)quinolin-2(1H)-one
84. 4,7-dimethyl-3-(1-propionyl-5-(p-tolyl)-4,5-dihydro-1H-pyrazol-3-yl)-1,8-naphthyridin-2(1H)-one
85. 4-methyl-3-(1-propionyl-5-(p-tolyl)-4,5-dihydro-1H-pyrazol-3-yl)-1,6-naphthyridin-2(1H)-one
86. 4-methyl-7-morpholino-3-(1-propionyl-5-(p-tolyl)-4,5-dihydro-1H-pyrazol-3-yl)-1,8-naphthyridin-2(1H)-one
87. 7-(2-methoxyethoxy)-4-methyl-3-(1-propionyl-5-(p-tolyl)-4,5-dihydro-1H-pyrazol-3-yl)-1,8-naphthyridin-2(1H)-one
or a pharmaceutically acceptable salt thereof or a pharmaceutically acceptable regioisomer thereof.

9. A novel bicyclic regioisomer compound selected from
(R)-5-(5-(4-methoxyphenyl)-1-propionyl-4,5-dihydro-1H-pyrazol-3-yl)-4-methylthieno[2,3-b]pyridin-6(7H)-one
(S)-5-(5-(4-methoxyphenyl)-1-propionyl-4,5-dihydro-1H-pyrazol-3-yl)-4-methylthieno[2,3-b]pyridin-6(7H)-one
(R)-5-(5-(4-chlorophenyl)-1-propionyl-4,5-dihydro-1H-pyrazol-3-yl)-4-methylthieno[2,3-b]pyridin-6(7H)-one
(S)-5-(5-(4-chlorophenyl)-1-propionyl-4,5-dihydro-1H-pyrazol-3-yl)-4-methylthieno[2,3-b]pyridin-6(7H)-one
(R)-4-methyl-5-(1-propionyl-5-(thiophen-3-yl)-4,5-dihydro-1H-pyrazol-3-yl)thieno[2,3-b]pyridin-6(7H)-one
(S)-4-methyl-5-(1-propionyl-5-(thiophen-3-yl)-4,5-dihydro-1H-pyrazol-3-yl)thieno[2,3-b]pyridin-6(7H)-one
(R)-4-methyl-5-(1-propionyl-5-(p-tolyl)-4,5-dihydro-1H-pyrazol-3-yl)thieno[2,3-b]pyridin-6(7H)-one
(S)-4-methyl-5-(1-propionyl-5-(p-tolyl)-4,5-dihydro-1H-pyrazol-3-yl)thieno[2,3-b]pyridin-6(7H)-one
(R)-4-methyl-5-(5-(6-methylpyridin-3-yl)-1-propionyl-4,5-dihydro-1H-pyrazol-3-yl)thieno[2,3-b]pyridin-6(7H)-one
(S)-4-methyl-5-(5-(6-methylpyridin-3-yl)-1-propionyl-4,5-dihydro-1H-pyrazol-3-yl)thieno[2,3-b]pyridin-6(7H)-one
(R)-4-methyl-3-(1-propionyl-5-(p-tolyl)-4,5-dihydro-1H-pyrazol-3-yl)quinolin-2(1H)-one
(S)-4-methyl-3-(1-propionyl-5-(p-tolyl)-4,5-dihydro-1H-pyrazol-3-yl)quinolin-2(1H)-one
(R)-5-(5-(2-fluoro-4-methylphenyl)-1-propionyl-4,5-dihydro-1H-pyrazol-3-yl)-4-methylthieno[2,3-b]pyridin-6(7H)-one
(S)-5-(5-(2-fluoro-4-methylphenyl)-1-propionyl-4,5-dihydro-1H-pyrazol-3-yl)-4-methylthieno[2,3-b]pyridin-6(7H)-one
(R)-4-methyl-3-(1-propionyl-5-(p-tolyl)-4,5-dihydro-1H-pyrazol-3-yl)-1,7-naphthyridin-2(1H)-one
(S)-4-methyl-3-(1-propionyl-5-(p-tolyl)-4,5-dihydro-1H-pyrazol-3-yl)-1,7-naphthyridin-2(1H)-one
(R)-4,8-dimethyl-3-(1-propionyl-5-(p-tolyl)-4,5-dihydro-1H-pyrazol-3-yl)-1,7-naphthyridin-2(1H)-one
(S)-4,8-dimethyl-3-(1-propionyl-5-(p-tolyl)-4,5-dihydro-1H-pyrazol-3-yl)-1,7-naphthyridin-2(1H)-one
(R)-7-chloro-4-methyl-3-(1-propionyl-5-(p-tolyl)-4,5-dihydro-1H-pyrazol-3-yl)quinolin-2(1H)-one
(S)-7-chloro-4-methyl-3-(1-propionyl-5-(p-tolyl)-4,5-dihydro-1H-pyrazol-3-yl)quinolin-2(1H)-one
(R)-4-methyl-7-(4-methylpiperazin-1-yl)-3-(1-propionyl-5-(p-tolyl)-4,5-dihydro-1H-pyrazol-3-yl)quinolin-2(1H)-one
(S)-4-methyl-7-(4-methylpiperazin-1-yl)-3-(1-propionyl-5-(p-tolyl)-4,5-dihydro-1H-pyrazol-3-yl)quinolin-2(1H)-one.

10. The pharmaceutical composition, comprising at least one compound according to claim 1, or a pharmaceutically acceptable salt or a stereoisomer thereof and a pharmaceutically acceptable carrier or excipient.

11. The compound according to claim 1, or a pharmaceutically acceptable salt or a stereoisomer thereof, for use as a medicament.

12. The method of inhibiting RAD51 protein comprising administering a therapeutically effective amount of a compound according to claim 1.

13. The method as claimed in claim 12 for the treatment of cancer associated with RAD51 protein.

14. The novel bicyclic compounds of formula (I) according to claim 4, wherein the ring C is specifically represented by

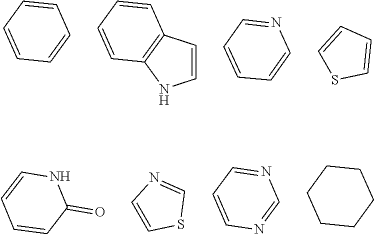

15. The novel bicyclic compounds of formula (I) according to claim 5, wherein the ring C is specifically represented by

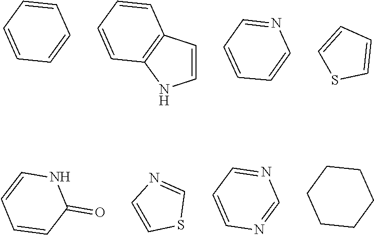

16. The novel bicyclic compounds of formula (I) according to claim 6, wherein the ring C is specifically represented by

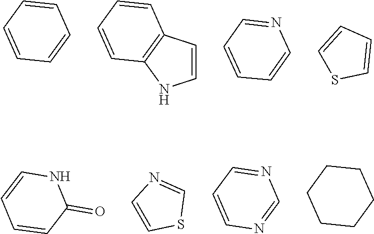

17. The pharmaceutical composition, comprising at least one compound according to claim 8, or a pharmaceutically acceptable salt or a stereoisomer thereof and a pharmaceutically acceptable carrier or excipient.

18. The compound according to claim 8, or a pharmaceutically acceptable salt or a stereoisomer thereof, for use as a medicament.

19. The method of inhibiting RAD51 protein comprising administering a therapeutically effective amount of a compound according to claim 8.

20. The method as claimed in claim 19 for the treatment of cancer associated with RAD51 protein.
